(19) United States
(12) Patent Application Publication
(10) Pub. No.: US 2010/0077513 A1
(43) Pub. Date: Mar. 25, 2010

(54) NOVEL PLANT TRANSCRIBED REGIONS AND USES THEREOF

(76) Inventors: Gregory J. Hinkle, Plymouth, MA (US); Jingdong Liu, Ballwin, MO (US); Linda T. Parker, Hackettstown, NJ (US)

Correspondence Address: ARNOLD & PORTER LLP, 555 TWELFTH STREET, N.W., ATTN: IP DOCKETING, WASHINGTON, DC, 20004, US

(21) Appl. No.: 12/621,344

(22) Filed: Nov. 18, 2009

Related U.S. Application Data
(63) Continuation of application No. 11/491,178, filed on Jul. 24, 2006.
Application No. 11/491,178 is a continuation of application No. 09/938,294, filed on Aug. 24, 2001, now abandoned.
(60) Provisional application No. 60/228,466, filed on Aug. 29, 2000.

(51) Int. Cl. A01H 5/00; C07H 21/04

(52) U.S. Cl. 800/298; 536/23.6

(57) ABSTRACT

The present invention is in the field of plant molecular biology. More specifically the invention relates to nucleic acid molecules that encode proteins and fragments of proteins. The invention also relates to proteins and fragments of proteins so encoded and antibodies capable of binding the proteins. The invention also relates to methods of using the nucleic acid molecules, proteins and fragments of proteins.

### CROSS REFERENCE TO RELATED APPLICATION

This application is a continuation of U.S. application Ser. No. 09/938,294 filed Aug. 24, 2001, which claims priority under 35 U.S.C. §119(e) to U.S. Application Ser. No. 60/228,466 filed Aug. 29, 2000, each of which is herein incorporated by reference in its entirety.

### INCORPORATION OF SEQUENCE LISTING

This application contains a computer-readable form of the sequence listing submitted herewith electronically via EFS web and contains the file named “P02079US03_seqlist.txt”, which is 285,168 bytes in size (measured in MS-DOS and Windows XP) and which was also created on Nov. 13, 2009, which is likewise herein incorporated by reference in its entirety.

### FIELD OF THE INVENTION

The present invention is in the field of plant molecular biology. More specifically the invention relates to nucleic acid molecules that encode proteins and fragments of proteins. The invention also relates to proteins and fragments of proteins so encoded and antibodies capable of binding the proteins. The invention also relates to methods of using the nucleic acid molecules, proteins and fragments of proteins.

### BACKGROUND OF THE INVENTION

The identification and isolation of novel plant genes are important in the development of nutritionally and agriculturally enhanced crops and products. High quality nucleic acid sequences with a low probability of base miss-calling of full length inserts, which often correspond to full length genes, provide a useful basis to develop nutritionally and agriculturally enhanced crops and products.

Nucleic acid molecules with high quality sequences can be used in a variety of applications. For example, novel coding nucleic acid molecules comprising coding sequences aid gene expression studies that allow the dissection and elucidation of commercially useful traits. Such expression approaches are particularly useful in determining function where the nucleic acid molecule fails to exhibit significant homology to other known nucleic acid molecules.

The present invention provides nucleic acid molecules with high fidelity nucleic acid sequences that do not exhibit significant homology with known nucleic acid sequences. The invention provides protein and fragment molecules with amino acid sequences that do not exhibit significant homology with known nucleic acid sequences. The nucleic acid molecules are drawn from maize, soybean and teosinte.

### SUMMARY OF THE INVENTION

The present invention includes and provides a substantially purified nucleic acid molecule comprising a nucleic acid sequence selected from SEQ ID NO: 1 through SEQ ID NO:43 or complements thereof or fragments of either.

The present invention further provides a substantially purified protein, peptide, or fragment thereof encoded by a nucleic acid sequence which specifically hybridizes to a nucleic acid molecule comprising a nucleic acid sequence selected from the group consisting of a complement of SEQ ID NO: 1 through SEQ ID NO: 43.

The present invention also provides a substantially purified protein or fragment thereof comprising an amino acid sequence selected from the group consisting of SEQ ID NO: 44 through SEQ ID NO 86 or fragment thereof.

The present invention also provides a substantially purified protein or fragment thereof encoded by a nucleic acid molecule comprising a nucleic acid sequence selected from the group consisting of SEQ ID NO: 1 through SEQ ID NO:43.

The present invention further provides a purified antibody or fragment thereof which is capable of specifically binding to a protein or fragment thereof, wherein the protein or fragment thereof comprises an amino acid sequence selected from the group consisting of SEQ ID NO:44 through SEQ ID NO: 86.

The present invention also provides a transformed plant having a nucleic acid molecule which comprises: (A) an exogenous promoter region which functions in a plant cell to cause the production of a mRNA molecule; (B) a structural nucleic acid molecule encoding a protein or fragment thereof comprising an amino acid sequence selected from the group consisting of SEQ ID NO: 44 through SEQ ID NO: 86 or fragment thereof; and (C) a 3′ non-translated sequence that functions in the plant cell to cause termination of transcription and addition of polyadenylated ribonucleotides to a 3′ end of the mRNA molecule.

The present invention also provides a transformed plant having a nucleic acid molecule which comprises: (A) an exogenous promoter region which functions in a plant cell to cause the production of a mRNA molecule; which is linked to (B) a transcribed nucleic acid molecule with a transcribed strand and a non-transcribed strand, wherein the transcribed strand is complementary to a nucleic acid molecule encoding a protein or fragment thereof comprising an amino acid sequence selected from the group consisting of SEQ ID NO: 44 through SEQ ID NO: 86 or fragment thereof; which is linked to (C) a 3′ non-translated sequence that functions in plant cells to cause termination of transcription and addition of polyadenylated ribonucleotides to a 3′ end of the mRNA molecule.

The present invention also provides a method for determining a level or pattern in a plant cell of a protein in a plant comprising: (A) incubating, under conditions permitting nucleic acid hybridization, a marker nucleic acid molecule, the marker nucleic acid molecule selected from the group of marker nucleic acid molecules which specifically hybridize to a nucleic acid molecule having the nucleic acid sequence selected from the group consisting of SEQ ID NO: 1 through SEQ ID NO: 43 or complements thereof or fragments of either, with a complementary nucleic acid molecule obtained from the plant cell or plant tissue, wherein nucleic acid hybridization between the marker nucleic acid molecule and the complementary nucleic acid molecule obtained from the plant cell or plant tissue permits the detection of an mRNA for the enzyme; (B) permitting hybridization between the marker nucleic acid molecule and the complementary nucleic acid molecule obtained from the plant cell or plant tissue; and (C) detecting the level or pattern of the complementary nucleic acid, wherein the detection of the complementary nucleic acid is predictive of the level or pattern of the protein.

The present invention also provides a method for determining the level or pattern of a protein in a plant cell or plant tissue comprising: (A) incubating under conditions permitting nucleic acid hybridization: a marker nucleic acid molecule, the marker nucleic acid molecule comprising a nucleotide sequence selected from the group consisting of SEQ ID NO: 1 through SEQ ID NO: 43 or complements thereof, with a complementary nucleic acid molecule obtained from a plant cell or plant tissue, wherein nucleic acid hybridization between the marker nucleic acid molecule, and the complementary nucleic acid molecule obtained from the plant cell or plant tissue permits the detection of said protein; (B) permitting hybridization between the marker nucleic acid molecule and the to complementary nucleic acid molecule obtained from the plant cell or plant tissue; and (C) detecting the level or pattern of the complementary nucleic acid, wherein the detection of said complementary nucleic acid is predictive of the level or pattern of the protein.

The present invention provides a method of determining a mutation in a plant whose presence is predictive of a mutation affecting a level or pattern of a protein comprising the steps: (A) incubating, under conditions permitting nucleic acid hybridization, a marker nucleic acid, the marker nucleic acid selected from the group of marker nucleic acid molecules which specifically hybridize to a nucleic acid molecule having a nucleic acid sequence selected from the group of SEQ ID NO: 1 through SEQ ID NO: 43 or complements thereof and a complementary nucleic acid molecule obtained from the plant, wherein nucleic acid hybridization between the marker nucleic acid molecule and the complementary nucleic acid molecule obtained from the plant permits the detection of a polymorphism whose presence is predictive of a mutation affecting the level or pattern of the protein in the plant; (B) permitting hybridization between the marker nucleic acid molecule and the complementary nucleic acid molecule obtained from the plant; and (C) detecting the presence of the polymorphism, wherein the detection of the polymorphism is predictive of the mutation.

The present invention also provides a method of producing a plant containing an overexpressed protein comprising: (A) transforming the plant with a functional nucleic acid molecule, wherein the functional nucleic acid molecule comprises a promoter region, wherein the promoter region is linked to a structural region, wherein the structural region comprises a nucleic acid sequence encoding an amino acid sequence selected from the group consisting of SEQ ID NO: 44 through SEQ ID NO: 86 or fragment thereof wherein the structural region is linked to a 3′ non-translated sequence that functions in the plant to cause termination of transcription and addition of polyadenylated ribonucleotides to a 3′ end of a mRNA molecule; and wherein the functional nucleic acid molecule results in overexpression of the protein; and (B) growing the transformed plant.

The present invention also provides a method of producing a plant containing reduced levels of a protein comprising: (A) transforming the plant with a functional nucleic acid molecule, wherein the functional nucleic acid molecule comprises a promoter region, wherein the promoter region is linked to a structural region, wherein the structural region comprises a nucleic acid molecule encoding an amino acid sequence consisting of SEQ ID NO: 44 through SEQ ID NO: 86 or fragment thereof; wherein the structural region is linked to a 3′ non-translated sequence that functions in the plant to cause termination of transcription and addition of polyadenylated ribonucleotides to a 3′ end of a mRNA molecule; and wherein the functional nucleic acid molecule results in co-suppression of the protein; and (B) growing the transformed plant.

The present invention also provides a method for reducing expression of a protein in a plant comprising: (A) transforming the plant with a nucleic acid molecule, the nucleic acid molecule having an exogenous promoter region which functions in a plant cell to cause the production of a mRNA molecule, wherein the exogenous promoter region is linked to a transcribed nucleic acid molecule having a transcribed strand and a non-transcribed strand, wherein the transcribed strand is complementary to a nucleic acid molecule having a nucleic acid sequence that encodes a protein having an amino acid sequence selected from the group consisting of SEQ ID NO: 44 through SEQ ID NO: 86 or fragments thereof and the transcribed strand is complementary to an endogenous mRNA molecule; and wherein the transcribed nucleic acid molecule is linked to a 3′ non-translated sequence that functions in the plant cell to cause termination of transcription and addition of polyadenylated ribonucleotides to a 3′ end of a mRNA molecule; and (B) growing the transformed plant.

The present invention also provides a method of determining an association between a polymorphism and a plant trait comprising: (A) hybridizing a nucleic acid molecule specific for the polymorphism to genetic material of a plant, wherein the nucleic acid molecule has a nucleic acid sequence selected from the group consisting of SEQ ID NO: 1 through SEQ ID NO: 43 or complements thereof or fragment of either; and (B) calculating the degree of association between the polymorphism and the plant trait.

The present invention also provides a method of isolating a nucleic acid that encodes a protein or fragment thereof comprising: (A) incubating under conditions permitting nucleic acid hybridization, a first nucleic acid molecule comprising a nucleic acid sequence selected from the group consisting of SEQ ID NO: 1 through SEQ ID NO:43 or complements thereof or fragment of either with a complementary second nucleic acid molecule obtained from a plant cell or plant tissue; (B) permitting hybridization between the first nucleic acid molecule and the second nucleic acid molecule obtained from the plant cell or plant tissue; and (C) isolating the second nucleic acid molecule.

The present invention also provides a method of analyzing the differences in the RNA profiles from more than one physiological source, said method comprising: a) obtaining a sample of ribonucleic acids from each of the physiological sources; b) generating a population of labeled nucleic acids for each of the physiological sources from said sample of ribonucleic acids; c) hybridizing the labeled nucleic acids for each of the physiological sources to an array of nucleic acid molecules stably associated with the surface of a substrate to produce a hybridization pattern for each of the physiological sources; said stably associated nucleic acid molecules selected from the group consisting of SEQ ID NO: 1 through SEQ ID NO: 43 or fragments thereof and d) comparing the hybridization patterns for each of the different physiological sources.

The present invention provides soybean, maize and teosinte nucleic acid molecules for use as molecular tags to isolate genetic regions (i.e. promoters and flanking sequences), isolate genes, map genes, and determine gene function. The present invention further provides soybean, maize and teosinte nucleic acid molecules for use in determining if genes are members of a particular gene family.

### DETAILED DESCRIPTION OF THE DRAWINGS

One skilled in the art can refer to general reference texts for detailed descriptions of known techniques discussed herein or equivalent techniques. These texts include Current Protocols in Molecular Biology Ausubel, et al., eds., John Wiley & Sons, N.Y. (1989), and supplements through September (1998), Molecular Cloning, A Laboratory Manual, Sambrook et al, 2^{nd} Ed., Cold Spring Harbor Press, Cold Spring Harbor, N.Y. (1989), Genome Analysis: A Laboratory Manual 1: Analyzing DNA, Birren et al., Cold Spring Harbor Press, Cold Spring Harbor, N.Y. (1997); Genome Analysis: A Laboratory Manual 2: Detecting Genes, Birren et al., Cold Spring Harbor Press, Cold Spring Harbor, N.Y. (1998); Genome Analysis: A Laboratory Manual 3: Cloning Systems, Birren et al., Cold Spring Harbor Press, Cold Spring Harbor, N.Y. (1999); Genome Analysis: A Laboratory Manual 4: Mapping Genomes, Birren et al., Cold Spring Harbor Press, Cold Spring Harbor, N.Y. (1999); Plant Molecular Biology: A Laboratory Manual, Clark, Springer-Verlag, Berlin, (1997), Methods in Plant Molecular Biology, Maliga et al., Cold Spring Harbor Press, Cold Spring Harbor, N.Y. (1995). These texts can, of course, also be referred to in making or using an aspect of the invention. It is understood that any of the agents of the invention can be substantially purified and/or be biologically active and/or recombinant.

Agents:

The agents of the invention will preferably be “biologically active” with respect to either a structural attribute, such as the capacity of a nucleic acid to hybridize to another nucleic acid molecule, or the ability of a protein to be bound by an antibody (or to compete with another molecule for such binding). Alternatively, such an attribute may be catalytic and thus involve the capacity of the agent to mediate a chemical reaction or response. The term “substantially purified”, as used herein, refers to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state

The agents of the invention may also be recombinant. As used herein, the term recombinant refers to a) molecules that are constructed outside of living cells by joining natural or synthetic DNA segments to DNA molecules that can replicate in a living cell or b) molecules that result from the replication or expression of those molecules described above or c) amino acid molecules from different sources which are joined together.

It is understood that the agents of the invention may be labeled with reagents that facilitate detection of the agent (e.g. fluorescent labels, Prober et al., Science 238:336-340 (1987); Albarella et al., EP 144914; chemical labels, Sheldon et al., U.S. Pat. No. 4,582,789; Albarella et al., U.S. Pat. No. 4,563,417; modified bases, Miyoshi et al., EP 119448). It is further understood that the invention provides recombinant bacterial, mammalian, microbial, archaebacterial, insect, fungal, algal, and plant cells as well as viral constructs comprising the agents of the invention.

### (a) Nucleic Acid Molecules

Agents of the invention include nucleic acid molecules and, more preferably, nucleic acid molecules of maize, soybean or teosinte. In addition, a number of different plants can be the ultimate source of the nucleic acid molecules of the invention. The type or strain of plant may not be particularly important, but an exemplary group of maize genotypes includes: B73 (Illinois Foundation Seeds, Champaign, Ill. U.S.A.); B73×Mo17 (Illinois Foundation Seeds, Champaign, Illinois U.S.A.); DK604 (Dekalb Genetics, Dekalb, Illinois U.S.A.); H99 (Illinois Foundation Seeds, Champaign, Illinois U.S.A.); RX601 (Asgrow Seed Company, Des Moines, Iowa); and Mo17 (Illinois Foundation Seeds, Champaign, Illinois U.S.A.). An exemplary group of soybean genotypes includes: Asgrow 3244 (Asgrow Seed Company, Des Moines, Iowa U.S.A.) and BW211S Null (Tohoku University, Morioka, Japan). An exemplary group of teosinte includes Zea mays L. ssp mexicana.

In one aspect of the present invention, the nucleic acid molecules have one or more of the nucleic acid sequences set forth in SEQ ID NO: 1 through SEQ ID NO: 43 or complements thereof or fragments of either.

One subset of the nucleic acid molecules of the invention is fragment nucleic acids molecules. Fragment nucleic acid molecules may consist of significant portion(s) of, or indeed most of, the nucleic acid molecules of the invention, such as those specifically disclosed. Alternatively, the fragments may comprise smaller oligonucleotides (having from about 15 to about 400 nucleotide residues and more preferably, about 15 to about 30 nucleotide residues, or about 50 to about 100 nucleotide residues, or about 100 to about 200 nucleotide residues, or about 200 to about 400 nucleotide residues, or about 275 to about 350 nucleotide residues).

A fragment of one or more of the nucleic acid molecules of the invention may be a probe and specifically a PCR probe. A PCR probe is a nucleic acid molecule capable of initiating a polymerase activity while in a double-stranded structure with another nucleic acid. Various methods for determining the structure of PCR probes and PCR techniques exist in the art. Computer generated searches using programs such as Primer3 (on the Worldwide web at genome.wi.mit.edu/cgi-bin/primer/primer3.cgi), STSPipeline (on the Worldwide web at genome.wi.mit.edu/cgi-bin/www-STS_Pipeline), or GeneUp (Pesole et al., BioTechniques 25:112-123 (1998)), for example, can be used to identify potential PCR primers.

Another subset of the nucleic acid molecules of the invention includes nucleic acid molecules that encode a protein or fragment thereof.

Nucleic acid molecules or fragments thereof of the present invention are capable of specifically hybridizing to other nucleic acid molecules under certain circumstances. Nucleic acid molecules of the present invention include those that specifically hybridize to nucleic acid molecules having a nucleic acid sequence selected from the group consisting of SEQ ID NO: 1 through SEQ ID NO: 43 or complements thereof.

As used herein, two nucleic acid molecules are said to be capable of specifically hybridizing to one another if the two molecules are capable of forming an anti-parallel, double-stranded nucleic acid structure.

A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989) and by Haymes et al., Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed.

Appropriate stringency conditions which promote DNA hybridization, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid molecules set forth in SEQ ID NO: 1 through SEQ ID NO: 43 or complements thereof under moderately stringent conditions, for example at about 2.0×SSC and about 65° C.

In a particularly preferred embodiment, a nucleic acid of the present invention will include those nucleic acid molecules that specifically hybridize to one or more of the nucleic acid molecules set forth in SEQ ID NO: 1 through SEQ ID NO: 43 or complements thereof under high stringency conditions such as 0.2×SSC and about 65° C.

In one aspect of the present invention, the nucleic acid molecules of the present invention have one or more of the nucleic acid sequences set forth in SEQ ID NO: 1 through SEQ ID NO: 43 or complements thereof. In another aspect of the present invention, one or more of the nucleic acid molecules of the present invention share between 100% and 90% sequence identity with one or more of the nucleic acid sequences set forth in SEQ ID NO: 1 through SEQ ID NO: 43 or complements thereof. In a further aspect of the present invention, one or more of the nucleic acid molecules of the present invention share between 100% and 95% sequence identity with one or more of the nucleic acid sequences set forth in SEQ ID NO: 1 through SEQ ID NO: 43 or complements thereof. In a more preferred aspect of the present invention, one or more of the nucleic acid molecules of the present invention share between 100% and 98% sequence identity with one or more of the nucleic acid sequences set forth in SEQ ID NO: 1 through SEQ ID NO: 43 or complements thereof. In an even more preferred aspect of the present invention, one or more of the nucleic acid molecules of the present invention share between 100% and 99% sequence identity with one or more of the sequences set forth in SEQ ID NO: 1 through SEQ ID NO: 43 or complements thereof.

The term “sequence identity” refers to the extent to which two sequences, nucleotide or amino acid, are invariant throughout the portion at which they are aligned. While there exist a number of methods to measure identity between two polynucleotide or polypeptide sequences, the term “sequence identity” is well known to skilled artisans. Methods commonly employed to determine identity between two sequences include, but are not limited to, those disclosed in Guide to Huge Computers, Martin J. Bishop, ed., Academic Press, San Diego, 1994, and Carillo, H., and Lipton, D., SIAM J Applied Math (1988) 48:1073. Methods to determine identity are codified in computer programs. Preferred computer program methods to determine identity between two sequences include, but are not limited to, the BLAST suite of programs publicly available from NCBI and other sources (BLAST Manual, Altschul et al., Natl. Cent. Biotechnol. Inf., Natl. Library Med. (NCBI NLM) NIH, Bethesda, Md. 20894; Altschul et al., J. Mol. Biol. 215:403-410 (1990), Pearson et al., Proc. Natl. Acad. Sci. U.S.A. 85:2444-2448 (1988), the FAST programs (Pearson et al., Proc. Natl. Acad. Sci. U.S.A. 85:2444-2448 (1988), the GAP and BESTFIT programs found in the GCG program package, (Madison, Wis.) and Cross_Match (Phi Green, University of Washington). Another preferred method to determine identity is by the method of DNASTAR protein alignment protocol using the Jotun-Hein algorithm (Hein et al., Methods Enzymol. 183:626-645 (1990)).

Unless otherwise noted, “percent sequence identity or percent identity” for this invention refers to the value obtained when using the BLAST 2.0 suite of programs with default parameters (Altschul et al., Nucleic Acids Res. 25:3389-3402, 1997; Altschul et al., J. Mol. Bio. 215: 403-410, 1990) Version 2.0 of BLAST allows the introduction of gaps (deletions and insertions) into alignments.

Nucleic acid molecules of the present invention can comprise sequences that encode a protein or fragment thereof. In a preferred aspect of the present invention the nucleic acid molecules encode an amino acid sequence consisting of SEQ ID: 44 through SEQ ID: 86 or fragment thereof.

Nucleic acid molecules of the present invention also include homologues. Particularly preferred homologues are selected from the group consisting of alfalfa, Arabidopsis, barley, Brassica, broccoli, cabbage, citrus, cotton, garlic, oat, oilseed rape, onion, canola, flax, an ornamental plant, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, soybean, maize, and Phaseolus.

In a preferred embodiment, nucleic acid molecules having SEQ ID NO: 1 through SEQ ID NO: 43 or complements thereof and fragments of either can be utilized to obtain such homologues.

In another further aspect of the present invention, nucleic acid molecules of the present invention can comprise sequences, which differ from those encoding a protein or fragment thereof in SEQ ID NO: 44 through SEQ ID NO: 86 due to fact that the different nucleic acid sequence encodes a protein having one or more conservative amino acid changes. It is understood that codons capable of coding for such conservative amino acid substitutions are known in the art.

It is well known in the art that one or more amino acids in a native sequence can be substituted with another amino acid(s), the charge and polarity of which are similar to that of the native amino acid, i.e., a conservative amino acid substitution, resulting in a silent change. Conserved substitutes for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to, (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, cystine, tyrosine, asparagine, and glutamine; and (4) neutral nonpolar (hydrophobic) amino acids such as alanine, leucine, isoleucine, valine, proline, phenylalanine, tryptophan, and methionine.

Conservative amino acid changes within the native polypeptide sequence can be made by substituting one amino acid within one of these groups with another amino acid within the same group. Biologically functional equivalents of the proteins or fragments thereof of the present invention can have ten or fewer conservative amino acid changes, more preferably seven or fewer conservative amino acid changes, and most preferably five or fewer conservative amino acid changes. The encoding nucleotide sequence will thus have corresponding base substitutions, permitting it to encode biologically functional equivalent forms of the proteins or fragments of the present invention.

It is understood that certain amino acids may be substituted for other amino acids in a protein structure without appreciable loss of interactive binding capacity with structures such as, for example, antigen-binding regions of antibodies or binding sites on substrate molecules. Because it is the interactive capacity and nature of a protein that defines that protein's biological functional activity, certain amino acid sequence substitutions can be made in a protein sequence and, of course, its underlying DNA coding sequence and, nevertheless, obtain a protein with like properties. It is thus contemplated by the inventors that various changes may be made in the peptide sequences of the proteins or fragments of the present invention, or corresponding DNA sequences that encode said peptides, without appreciable loss of their biological utility or activity. It is understood that codons capable of coding for such amino acid changes are known in the art.

In making such changes, the hydropathic index of amino acids may be considered. The importance of the hydropathic amino acid index in conferring interactive biological function on a protein is generally understood in the art (Kyte and Doolittle, J. Mol. Biol. 157, 105-132 (1982)). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of its hydrophobicity and charge characteristics (Kyte and Doolittle, J. Mol. Biol. 157, 105-132 (1982)); these are isoleucine (+4.5), valine (+4.2), leucine (+3.8), phenylalanine (+2.8), cysteine/cystine (+2.5), methionine (+1.9), alanine (+1.8), glycine (−0.4), threonine (−0.7), serine (−0.8), tryptophan (−0.9), tyrosine (−1.3), proline (−1.6), histidine (−3.2), glutamate (−3.5), glutamine (−3.5), aspartate (−3.5), asparagine (−3.5), lysine (−3.9), and arginine (−4.5).

In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101 states that the greatest local average hydrophilicity of a protein, as govern by the hydrophilicity of its adjacent amino acids, correlates with a biological property of the protein.

As detailed in U.S. Pat. No. 4,554,101, the following hydrophilicity values have been assigned to amino acid residues: arginine (+3.0), lysine (+3.0), aspartate (+3.0±1), glutamate (+3.0±1), serine (+0.3), asparagine (+0.2), glutamine (+0.2), glycine (0), threonine (−0.4), proline (−0.5±1), alanine (−0.5), histidine (−0.5), cysteine (−1.0), methionine (−1.3), valine (−1.5), leucine (−1.8), isoleucine (−1.8), tyrosine (−2.3), phenylalanine (−2.5), and tryptophan (−3.4).

In making such changes, the substitution of amino acids whose hydrophilicity values are within ±2 is preferred, those which are within ±1 are particularly preferred, and those within ±0.5 are even more particularly preferred.

In a further aspect of the present invention, one or more of the nucleic acid molecules of the present invention differ in nucleic acid sequence from those encoding a protein or fragment thereof set forth in SEQ ID NO: 1 through SEQ ID NO: 43 or fragment thereof due to the fact that one or more codons encoding an amino acid has been substituted for a codon that encodes a nonessential substitution of the amino acid originally encoded.

Agents of the invention include nucleic acid molecules that encode at least about a contiguous 10 amino acid region of a protein of the present invention, more preferably at least about a contiguous 25, 40, 50, 100, or 125 amino acid region of a protein of the present invention

A nucleic acid molecule of the invention can also encode a homologue protein. As used herein, a homologue protein molecule or fragment thereof is a counterpart protein molecule or fragment thereof in a second species (e.g., maize transcription factor AP2 is a homologue of Arabidopsis transcription factor AP2). A homologue can also be generated by molecular evolution or DNA shuffling techniques, so that the molecule retains at least one functional or structure characteristic of the original protein (see, for example, U.S. Pat. No. 5,811,238).

### (b) Protein and Peptide Molecules

A class of agents includes one or more of the protein or fragments thereof or peptide molecules having a nucleic acid sequence selected from the group consisting of SEQ ID NO:1 through SEQ ID NO: 43 or one or more of the protein or fragment thereof and peptide molecules encoded by other nucleic acid agents of the invention. A particular preferred class of proteins are those having an amino acid sequence selected from the group consisting of SEQ ID NO: 44 through SEQ ID NO: 86 or fragments thereof.

As used herein, the term “protein molecule” or “peptide molecule” includes any molecule that comprises five or more amino acids. It is well known in the art that proteins may undergo modification, including post-translational modifications, such as, but not limited to, disulfide bond formation, glycosylation, phosphorylation, or oligomerization. Thus, as used herein, the term “protein molecule” or “peptide molecule” includes any protein molecule that is modified by any biological or non-biological process. The terms “amino acid” and “amino acids” refer to all naturally occurring L-amino acids. This definition is meant to include norleucine, norvaline, ornithine, homocysteine, and homoserine.

One or more of the protein or fragment of peptide molecules may be produced via chemical synthesis, or more preferably, by expressing in a suitable bacterial or eukaryotic host. Suitable methods for expression are described by Sambrook et al., In: Molecular Cloning, A Laboratory Manual, 2nd Edition, Cold Spring Harbor Press, Cold Spring Harbor, N.Y. (1989), or similar texts.

A “protein fragment” is a peptide or polypeptide molecule whose amino acid sequence comprises a subset of the amino acid sequence of that protein. A protein or fragment thereof that comprises one or more additional peptide regions not derived from that protein is a “fusion” protein. Such molecules may be derivatized to contain carbohydrate or other moieties (such as keyhole limpet hemocyanin, etc.). Fusion protein or peptide molecules of the invention are preferably produced via recombinant means.

Another class of agents comprise protein or peptide molecules or fragments or fusions thereof comprising SEQ ID NO: 44 through SEQ ID NO: 86 or fragment thereof or encoded by SEQ ID NO: 1 through SEQ ID NO: 43 in which conservative, non-essential or non-relevant amino acid residues have been added, replaced or deleted. Computerized means for designing modifications in protein structure are known in the art (Dahiyat and Mayo, Science 278:82-87 (1997)).

Protein molecules of the present invention also include homologues. Particularly preferred homologues are selected from the group consisting of alfalfa, Arabidopsis, barley, Brassica, broccoli, cabbage, citrus, cotton, garlic, oat, oilseed rape, onion, canola, flax, an ornamental plant, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, maize, soybean, and Phaseolus.

In a preferred embodiment, nucleic acid molecules having SEQ ID NO: 1 through SEQ ID NO: 43 or complements and fragments of either can be utilized to obtain such homologues.

The degeneracy of the genetic code, which allows different nucleic acid sequences to code for the same protein or peptide, is known in the literature (U.S. Pat. No. 4,757,006).

In another further aspect of the present invention, one or more of the protein molecules of the present invention differ in protein sequence from those set forth in SEQ ID NO: 44 through SEQ ID NO: 86 or fragment thereof due to fact that the different protein encodes a protein having one or more conservative amino acid residue. In a further aspect of the present invention, one or more of the protein molecules of the present invention differ in protein sequence from those set forth in SEQ ID NO: 44 through SEQ ID NO: 86 or fragment thereof due to the fact that one or more codons encoding an amino acid has been substituted for a codon that encodes a nonessential substitution of the amino acid originally encoded.

Agents of the invention include proteins comprising at least about a contiguous 10 amino acid region preferably comprising at least about a contiguous 20 amino acid region, even more preferably comprising at least a contiguous 25, 35, 50, 75 or 100 amino acid region of a protein or fragment thereof of the present invention. In another preferred embodiment, the proteins of the present invention include a between about 10 and about 25 contiguous amino acid region, more preferably between about 20 and about 50 contiguous amino acid region and even more preferably a between about 40 and about 80 contiguous amino acid region.

In another preferred embodiment, the protein comprises an amino acid sequence selected from the group consisting of SEQ ID NO: 44 through SEQ ID NO: 86.

A protein of the invention can also be a homologue protein. A homologue can also be generated by molecular evolution or DNA shuffling techniques, so that the molecule retains at least one functional or structure characteristic of the original (see, for example, U.S. Pat. No. 5,811,238).

Protein molecules of the present invention include homologues of proteins or fragments thereof comprising a protein sequence selected from SEQ ID NO: 44 through SEQ ID NO: 86, or fragment thereof or encoded by SEQ ID NO:1 through SEQ ID NO: 43 or fragments thereof. Preferred protein molecules of the invention include homologues of proteins or fragments having an amino acid sequence selected from the group consisting of SEQ ID NO: 44 through SEQ ID NO: 86 or fragment thereof. A homologue protein may be derived from, but not limited to, alfalfa, Arabidopsis, barley, Brassica, broccoli, cabbage, citrus, cotton, garlic, oat, oilseed rape, onion, canola, flax, an ornamental plant, pea, peanut, pepper, potato, rice, rye, sorghum, strawberry, sugarcane, sugarbeet, tomato, wheat, poplar, pine, fir, eucalyptus, apple, lettuce, lentils, grape, banana, tea, turf grasses, sunflower, oil palm, maize, soybean Phaseolus etc. Particularly preferred species for use in the isolation of homologs would include, Arabidopsis, barley, cotton, oat, oilseed rape, rice, canola, ornamentals, sugarcane, sugarbeet, tomato, potato, wheat and turf grasses. Such a homologue can be obtained by any of a variety of methods. Most preferably, as indicated above, one or more of the disclosed sequences (such as SEQ ID NO: 1 through SEQ ID NO:43 or complements thereof) will be used in defining a pair of primers to isolate the homologue-encoding nucleic acid molecules from any desired species. Such molecules can be expressed to yield protein homologues by recombinant means.

### (c) Plant Constructs and Plant Transformants

One or more of the nucleic acid molecules of the invention may be used in plant transformation or transfection. Exogenous genetic material may be transferred into a plant cell and the plant cell regenerated into a whole, fertile or sterile plant. Exogenous genetic material is any genetic material, whether naturally occurring or otherwise, from any source that is capable of being inserted into any organism. In a preferred embodiment the exogenous genetic material includes a nucleic acid molecule of the present invention, preferably a nucleic acid molecule having a sequence selected from the group consisting of SEQ ID NO: 1 through SEQ ID NO: 43 or complements thereof or fragments of either. Another preferred class of exogenous genetic material are nucleic acid molecules that encode a protein or fragment thereof having an amino acid selected from the group consisting of SEQ ID NO: 44 through SEQ ID NO: 86 or fragments thereof.

Such genetic material may be transferred into either monocotyledons and dicotyledons including, but not limited to maize, soybean, Arabidopsis, phaseolus, peanut, alfalfa, wheat, rice, oat, sorghum, rye, tritordeum, millet, fescue, perennial ryegrass, sugarcane, cranberry, papaya, banana, banana, muskmelon, apple, cucumber, dendrobium, gladiolus, chrysanthemum, liliacea, cotton, eucalyptus, sunflower, canola, turfgrass, sugarbeet, coffee and dioscorea (Christou, In: Particle Bombardment for Genetic Engineering of Plants, Biotechnology Intelligence Unit, Academic Press, San Diego, Calif. (1996)).

Transfer of a nucleic acid that encodes for a protein can result in overexpression of that protein in a transformed cell or transgenic plant. One or more of the proteins or fragments thereof encoded by nucleic acid molecules of the invention may be overexpressed in a transformed cell or transformed plant. Such overexpression may be the result of transient or stable transfer of the exogenous genetic material.

Exogenous genetic material may be transferred into a host cell by the use of a DNA vector or construct designed for such a purpose. Design of such a vector is generally within the skill of the art (See, Plant Molecular Biology: A Laboratory Manual, Clark (ed.), Springier, New York (1997)).

A construct or vector may include a plant promoter to express the protein or protein fragment of choice. A number of promoters, which are active in plant cells, have been described in the literature. These include the nopaline synthase (NOS) promoter (Ebert et al., Proc. Natl. Acad. Sci. (U.S.A.) 84:5745-5749 (1987)), the octopine synthase (OCS) promoter (which are carried on tumor-inducing plasmids of Agrobacterium tumefaciens), the caulimovirus promoters such as the cauliflower mosaic virus (CaMV) 19S promoter (Lawton et al., Plant Mol. Biol. 9:315-324 (1987)) and the CaMV 35S promoter (Odell et al., Nature 313:810-812 (1985)), the figwort mosaic virus 35S-promoter, the light-inducible promoter from the small subunit of ribulose-1,5-bis-phosphate carboxylase (ssRUBISCO), the Adh promoter (Walker et al., Proc. Natl. Acad. Sci. (U.S.A.) 84:6624-6628 (1987)), the sucrose synthase promoter (Yang et al., Proc. Natl. Acad. Sci. (U.S.A.) 87:4144-4148 (1990)), the R gene complex promoter (Chandler et al., The Plant Cell 1:1175-1183 (1989)) and the chlorophyll a/b binding protein gene promoter, etc. These promoters have been used to create DNA constructs that have been expressed in plants; see, e.g., PCT publication WO 84/02913. The CaMV 35S promoters are preferred for use in plants. Promoters known or found to cause transcription of DNA in plant cells can be used in the invention.

For the purpose of expression in source tissues of the plant, such as the leaf, seed, root or stem, it is preferred that the promoters utilized have relatively high expression in these specific tissues. Tissue-specific expression of a protein of the present invention is a particularly preferred embodiment. For this purpose, one may choose from a number of promoters for genes with tissue- or cell-specific or -enhanced expression. Examples of such promoters reported in the literature include the chloroplast glutamine synthetase GS2 promoter from pea (Edwards et al., Proc. Natl. Acad. Sci. (U.S.A.) 87:3459-3463 (1990)), the chloroplast fructose-1,6-biphosphatase (FBPase) promoter from wheat (Lloyd et al., Mol. Gen. Genet. 225:209-216 (1991)), the nuclear photosynthetic ST-LS1 promoter from potato (Stockhaus et al., EMBO J. 8:2445-2451 (1989)), the serine/threonine kinase (PAL) promoter and the glucoamylase (CHS) promoter from Arabidopsis thaliana. Also reported to be active in photosynthetically active tissues are the ribulose-1,5-bisphosphate carboxylase (RbcS) promoter from eastern larch (Larix laricina), the promoter for the cab gene, cab6, from pine (Yamamoto et al., Plant Cell Physiol. 35:773-778 (1994)), the promoter for the Cab-1 gene from wheat (Fejes et al., Plant Mol. Biol. 15:921-932 (1990)), the promoter for the CAB-1 gene from spinach (Lubberstedt et al., Plant Physiol. 104:997-1006 (1994)), the promoter for the cab1R gene from rice (Luan et al., Plant Cell. 4:971-981 (1992)), the pyruvate, orthophosphate dikinase (PPDK) promoter from maize (Matsuoka et al., Proc. Natl. Acad. Sci. (U.S.A.) 90: 9586-9590 (1993)), the promoter for the tobacco Lhcb1*2 gene (Cerdan et al., Plant Mol. Biol. 33:245-255 (1997)), the Arabidopsis thaliana SUC2 sucrose-H+ symporter promoter (Truernit et al., Planta. 196:564-570 (1995)) and the promoter for the thylakoid membrane proteins from spinach (psaD, psaF, psaE, PC, FNR, atpC, atpD, cab, rbcS). Other promoters for the chlorophyll a/b-binding proteins may also be utilized in the invention, such as the promoters for LhcB gene and PsbP gene from white mustard (Sinapis alba; Kretsch et al., Plant Mol. Biol. 28:219-229 (1995)).

For the purpose of expression in sink tissues of the plant, such as the tuber of the potato plant, the fruit of tomato, or the seed of maize, wheat, rice and barley, it is preferred that the promoters utilized in the invention have relatively high expression in these specific tissues. A number of promoters for genes with tuber-specific or -enhanced expression are known, including the class I patatin promoter (Bevan et al., EMBO J. 8:1899-1906 (1986); Jefferson et al., Plant Mol. Biol. 14:995-1006 (1990)), the promoter for the potato tuber ADPGPP genes, both the large and small subunits, the sucrose synthase promoter (Salanoubat and Belliard, Gene 60:47-56 (1987), Salanoubat and Belliard, Gene 84:181-185 (1989)), the promoter for the major tuber proteins including the 22 kd protein complexes and proteinase inhibitors (Hannapel, Plant Physiol. 101:703-704 (1993)), the promoter for the granule bound starch synthase gene (GBSS) (Visser et al., Plant Mol. Biol. 17:691-699 (1991)) and other class I and II patatins promoters (Koster-Topfer et al., Mol Gen Genet. 219:390-396 (1989); Mignery et al., Gene. 62:27-44 (1988)).

Other promoters can also be used to express a protein or fragment thereof in specific tissues, such as seeds or fruits. The promoter for β-conglycinin (Chen et al., Dev. Genet. 10: 112-122 (1989)) or other seed-specific promoters, such as the napin and phaseolin promoters, can be used. The zeins are a group of storage proteins found in maize endosperm. Genomic clones for zein genes have been isolated (Pedersen et al., Cell 29:1015-1026 (1982)) and the promoters from these clones, including the 15 kD, 16 kD, 19 kD, 22 kD, 27 kD and genes, could also be used. Other promoters known to function, for example, in maize include the promoters for the following genes: waxy, Brittle, Shrunken 2, Branching enzymes I and II, starch synthases, debranching enzymes, oleosins, glutelins and sucrose synthases. A particularly preferred promoter for maize endosperm expression is the promoter for the glutelin gene from rice, more particularly the Osgt-1 promoter (Zheng et al., Mol. Cell Biol. 13:5829-5842 (1993)). Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins and the glutenins. Examples of such promoters in rice include those promoters for the ADPGPP subunits, the granule bound and other starch synthase, the branching enzymes, the debranching enzymes, sucrose synthases and the glutelins. A particularly preferred promoter is the promoter for rice glutelin, Osgt-1. Examples of such promoters for barley include those for the ADPGPP subunits, the granule bound and other starch synthase, the branching enzymes, the debranching enzymes, sucrose synthases, the hordeins, the embryo globulins and the aleurone specific proteins.

Root specific promoters may also be used. An example of such a promoter is the promoter for the acid chitinase gene (Samac et al., Plant Mol. Biol. 25:587-596 (1994)). Expression in root tissue could also be accomplished by utilizing the root specific subdomains of the CaMV35S promoter that have been identified (Lam et al., Proc. Natl. Acad. Sci. (U.S.A.) 86:7890-7894 (1989)). Other root cell specific promoters include those reported by Conkling et al. (Conkling et al., Plant Physiol. 93:1203-1211 (1990)).

Additional promoters that may be utilized are described, for example, in U.S. Pat. Nos. 5,378,619; 5,391,725; 5,428,147; 5,447,858; 5,608,144; 5,608,144; 5,614,399; 5,633,441; 5,633,435; and 4,633,436. In addition, a tissue specific enhancer may be used (Fromm et al., The Plant Cell 1:977-984 (1989)).

Constructs or vectors may also include, with the coding region of interest, a nucleic acid sequence that acts, in whole or in part, to terminate transcription of that region. A number of such sequences have been isolated, including the Tr7 3′ sequence and the NOS 3′ sequence (Ingelbrecht et al., The Plant Cell 1:671-680 (1989); Bevan et al., Nucleic Acids Res. 11:369-385 (1983)).

A vector or construct may also include regulatory elements. Examples of such include the Adh intron 1 (Callis et al., Genes and Develop. 1:1183-1200 (1987)), the sucrose synthase intron (Vasil et al., Plant Physiol. 91:1575-1579 (1989)) and the TMV omega element (Gallie et al., The Plant Cell 1:301-311 (1989)). These and other regulatory elements may be included when appropriate.

A vector or construct may also include a selectable marker. Selectable markers may also be used to select for plants or plant cells that contain the exogenous genetic material. Examples of such include, but are not limited to: a neo gene (Potrykus et al., Mol. Gen. Genet. 199:183-188 (1985)), which codes for kanamycin resistance and can be selected for using kanamycin, G418, etc.; a bar gene which codes for bialaphos resistance; a mutant EPSP synthase gene (Hinchee et al., Bio/Technology 6:915-922 (1988)) which encodes glyphosate resistance; a nitrilase gene which confers resistance to bromoxynil (Stalker et al., J. Biol. Chem. 263:6310-6314 (1988)); a mutant acetolactate synthase gene (ALS) which confers imidazolinone or sulphonylurea resistance (European Patent Application 154,204 (Sep. 11, 1985)); and a methotrexate resistant DHFR gene (Thillet et al., J. Biol. Chem. 263:12500-12508 (1988)).

A vector or construct may also include a transit peptide. Incorporation of a suitable chloroplast transit peptide may also be employed (European Patent Application Publication Number 0218571). Translational enhancers may also be incorporated as part of the vector DNA. DNA constructs could contain one or more 5′ non-translated leader sequences that may serve to enhance expression of the gene products from the resulting mRNA transcripts. Such sequences may be derived from the promoter selected to express the gene or can be specifically modified to increase translation of the mRNA. Such regions may also be obtained from viral RNAs, from suitable eukaryotic genes, or from a synthetic gene sequence. For a review of optimizing expression of transgenes, see Koziel et al., Plant Mol. Biol. 32:393-405 (1996).

A vector or construct may also include a screenable marker. Screenable markers may be used to monitor expression. Exemplary screenable markers include: a (3-glucuronidase or uidA gene (GUS) which encodes an enzyme for which various chromogenic substrates are known (Jefferson, Plant Mol. Biol, Rep. 5:387-405 (1987); Jefferson et al., EMBO J. 6:3901-3907 (1987)); an R-locus gene, which encodes a product that regulates the production of anthocyanin pigments (red color) in plant tissues (Dellaporta et al., Stadler Symposium 11:263-282 (1988)); a β-lactamase gene (Sutcliffe et al., Proc. Natl. Acad. Sci. (U.S.A.) 75:3737-3741 (1978)), a gene which encodes an enzyme for which various chromogenic substrates are known (e.g., PADAC, a chromogenic cephalosporin); a luciferase gene (Ow et al., Science 234:856-859 (1986)); a xylE gene (Zukowsky et al., Proc. Natl. Acad. Sci. (U.S.A.) 80:1101-1105 (1983)) which encodes a catechol dioxygenase that can convert chromogenic catechols; an α-amylase gene (Ikatu et al., Bio/Technol. 8:241-242 (1990)); a tyrosinase gene (Katz et al., J. Gen. Microbiol. 129:2703-2714 (1983)) which encodes an enzyme capable of oxidizing tyrosine to DOPA and dopaquinone which in turn condenses to melanin; an α-galactosidase, which will turn a chromogenic α-galactose substrate.

Included within the terms “selectable or screenable marker genes” are also genes that encode a secretable marker whose secretion can be detected as a means of identifying or selecting for transformed cells. Examples include markers that encode a secretable antigen that can be identified by antibody interaction, or even secretable enzymes that can be detected catalytically. Secretable proteins fall into a number of classes, including small, diffusible proteins which are detectable, (e.g., by ELISA), small active enzymes which are detectable in extracellular solution (e.g., α-amylase, β-lactamase, phosphinothricin transferase), or proteins which are inserted or trapped in the cell wall (such as proteins which include a leader sequence such as that found in the expression unit of extension or tobacco PR—S). Other possible selectable and/or screenable marker genes will be apparent to those of skill in the art.

There are many methods for introducing transforming nucleic acid molecules into plant cells. Suitable methods are believed to include virtually any method by which nucleic acid molecules may be introduced into a cell, such as by Agrobacterium infection or direct delivery of nucleic acid molecules such as, for example, by PEG-mediated transformation, by electroporation or by acceleration of DNA coated particles, etc (Potrykus, Ann. Rev. Plant Physiol. Plant Mol. Biol. 42:205-225 (1991); Vasil, Plant Mol. Biol. 25:925-937 (1994)). For example, electroporation has been used to transform maize protoplasts (Fromm et al., Nature 312:791-793 (1986)).

Other vector systems suitable for introducing transforming DNA into a host plant cell include but are not limited to binary artificial chromosome (BIBAC) vectors (Hamilton et al., Gene 200:107-116 (1997)); and transfection with RNA viral vectors (Della-Cioppa et al., Ann. N.Y. Acad. Sci. (1996), 792 (Engineering Plants for Commercial Products and Applications), 57-61). Additional vector systems also include plant selectable YAC vectors such as those described in Mullen et al., Molecular Breeding 4:449-457 (1988)).

Technology for introduction of DNA into cells is well known to those of skill in the art. Four general methods for delivering a gene into cells have been described: (1) chemical methods (Graham and van der Eb, Virology 54:536-539 (1973)); (2) physical methods such as microinjection (Capecchi, Cell 22:479-488 (1980)), electroporation (Wong and Neumann, Biochem. Biophys. Res. Commun. 107:584-587 (1982); Fromm et al., Proc. Natl. Acad. Sci. (U.S.A.) 82:5824-5828 (1985); U.S. Pat. No. 5,384,253); and the gene gun (Johnston and Tang, Methods Cell Biol. 43:353-365 (1994)); (3) viral vectors (Clapp, Clin. Perinatol. 20:155-168 (1993); Lu et al., J. Exp. Med. 178:2089-2096 (1993); Eglitis and Anderson, Biotechniques 6:608-614 (1988)); and (4) receptor-mediated mechanisms (Curiel et al., Hum. Gen. Ther. 3:147-154 (1992), Wagner et al., Proc. Natl. Acad. Sci. (USA) 89:6099-6103 (1992)).

Acceleration methods that may be used include, for example, microprojectile bombardment and the like. One example of a method for delivering transforming nucleic acid molecules to plant cells is microprojectile bombardment. This method has been reviewed by Yang and Christou (eds.), Particle Bombardment Technology for Gene Transfer, Oxford Press, Oxford, England (1994)). Non-biological particles (microprojectiles) that may be coated with nucleic acids and delivered into cells by a propelling force. Exemplary particles include those comprised of tungsten, gold, platinum and the like.

A particular advantage of microprojectile bombardment, in addition to it being an effective means of reproducibly transforming monocots, is that neither the isolation of protoplasts (Cristou et al., Plant Physiol. 87:671-674 (1988)) nor the susceptibility of Agrobacterium infection are required. An illustrative embodiment of a method for delivering DNA into maize cells by acceleration is a biolistics α-particle delivery system, which can be used to propel particles coated with DNA through a screen, such as a stainless steel or Nytex screen, onto a filter surface covered with corn cells cultured in suspension. Gordon-Kamm et al., describes the basic procedure for coating tungsten particles with DNA (Gordon-Kamm et al., Plant Cell 2:603-618 (1990)). The screen disperses the tungsten nucleic acid particles so that they are not delivered to the recipient cells in large aggregates. A particle delivery system suitable for use with the invention is the helium acceleration PDS-1000/He gun is available from Bio-Rad Laboratories (Bio-Rad, Hercules, Calif.)(Sanford et al., Technique 3:3-16 (1991)).

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein one may obtain up to 1000 or more foci of cells transiently expressing a marker gene. The number of cells in a focus which express the exogenous gene product 48 hours post-bombardment often range from one to ten and average one to three.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include the particle gun delivery of DNA containing a selectable marker and targeting of the DNA to the plastid genome through homologous recombination (Svab et al., Proc. Natl. Acad. Sci. (U.S.A.) 87:8526-8530 (1990); Svab and Maliga, Proc. Natl. Acad. Sci. (U.S.A.) 90:913-917 (1993); Staub and Maliga, EMBO J. 12:601-606 (1993); U.S. Pat. Nos. 5,451,513 and 5,545,818).

Accordingly, it is contemplated that one may wish to adjust various aspects of the bombardment parameters in small-scale studies to fully optimize the conditions. One may particularly wish to adjust physical parameters such as gap distance, flight distance, tissue distance and helium pressure. One may also minimize the trauma reduction factors by modifying conditions which influence the physiological state of the recipient cells and which may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art. See, for example the methods described by Fraley et al., Bio/Technology 3:629-635 (1985) and Rogers et al., Methods Enzymol. 153:253-277 (1987). Further, the integration of the Ti-DNA is a relatively precise process resulting in few rearrangements. The region of DNA to be transferred is defined by the border sequences and intervening DNA is usually inserted into the plant genome as described (Spielmann et al., Mol. Gen. Genet. 205:34 (1986)).

Modern Agrobacterium transformation vectors are capable of replication in E. coli as well as Agrobacterium, allowing for convenient manipulations as described (Klee et al., In: Plant DNA Infectious Agents, Hohn and Schell (eds.), Springer-Verlag, New York, pp. 179-203 (1985)). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes (Rogers et al., Methods Enzymol. 153:253-277 (1987)). In addition, Agrobacterium containing both armed and disarmed Ti genes can be used for the transformations. In those plant strains where Agrobacterium-mediated transformation is efficient, it is the method of choice because of the facile and defined nature of the gene transfer.

A transgenic plant formed using Agrobacterium transformation methods typically contains a single gene on one chromosome. Such transgenic plants can be referred to as being heterozygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants produced for the gene of interest.

It is also to be understood that two different transgenic plants can also be mated to produce offspring that contain two independently segregating, exogenous genes. Selfing of appropriate progeny can produce plants that are homozygous for both added, exogenous genes that encode a polypeptide of interest. Backcrossing to a parental plant and out-crossing with a non-transgenic plant are also contemplated, as is vegetative propagation.

Transformation of plant protoplasts can be achieved using methods based on calcium phosphate precipitation, polyethylene glycol treatment, electroporation and combinations of these treatments (See, for example, Potrykus et al., Mol. Gen. Genet. 205:193-200 (1986); Lorz et al., Mol. Gen. Genet. 199:178 (1985); Fromm et al., Nature 319:791 (1986); Uchimiya et al., Mol. Gen. Genet. 204:204 (1986); Marcotte et al., Nature 335:454-457 (1988)).

Application of these systems to different plant strains depends upon the ability to regenerate that particular plant strain from protoplasts. Illustrative methods for the regeneration of cereals from protoplasts are described (Fujimura et al., Plant Tissue Culture Letters 2:74 (1985); Toriyama et al., Theor Appl. Genet. 205:34 (1986); Yamada et al., Plant Cell Rep. 4:85 (1986); Abdullah et al., Biotechnology 4:1087 (1986)).

To transform plant strains that cannot be successfully regenerated from protoplasts, other ways to introduce DNA into intact cells or tissues can be utilized. For example, regeneration of cereals from immature embryos or explants can be effected as described (Vasil, Biotechnology 6:397 (1988)). In addition, “particle gun” or high-velocity microprojectile technology can be utilized (Vasil et al., Bio/Technology 10:667 (1992)).

Using the latter technology, DNA is carried through the cell wall and into the cytoplasm on the surface of small metal particles as described (Klein et al., Nature 328:70 (1987); Klein et al., Proc. Natl. Acad. Sci. (U.S.A.) 85:8502-8505 (1988); McCabe et al., Bio/Technology 6:923 (1988)). The metal particles penetrate through several layers of cells and thus allow the transformation of cells within tissue explants.

Other methods of cell transformation can also be used and include but are not limited to introduction of DNA into plants by direct DNA transfer into pollen (Hess et al., Intern Rev. Cytol. 107:367 (1987); Luo et al., Plant Mol. Biol. Reporter 6:165 (1988)), by direct injection of DNA into reproductive organs of a plant (Pena et al., Nature 325:274 (1987)), or by direct injection of DNA into the cells of immature embryos followed by the rehydration of desiccated embryos (Neuhaus et al., Theor. Appl. Genet. 75:30 (1987)). The regeneration, development and cultivation of plants from single plant protoplast transformants or from various transformed explants are well known in the art (Weissbach and Weissbach, In: Methods for Plant Molecular Biology, Academic Press, San Diego, Calif., (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene that encodes a protein of interest is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the invention containing a desired polypeptide is cultivated using methods well known to one skilled in the art.

There are a variety of methods for the regeneration of plants from plant tissue. The particular method of regeneration will depend on the starting plant tissue and the particular plant species to be regenerated.

Methods for transforming dicots, primarily by use of Agrobacterium tumefaciens and obtaining transgenic plants have been published for cotton (U.S. Pat. No. 5,004,863; U.S. Pat. No. 5,159,135; U.S. Pat. No. 5,518,908); soybean (U.S. Pat. No. 5,569,834; U.S. Pat. No. 5,416,011; McCabe et al., Biotechnology 6:923 (1988); Christou et al., Plant Physiol. 87:671-674 (1988)); Brassica (U.S. Pat. No. 5,463,174); peanut (Cheng et al., Plant Cell Rep. 15:653-657 (1996), McKently et al., Plant Cell Rep. 14:699-703 (1995)); papaya; and pea (Grant et al., Plant Cell Rep. 15:254-258 (1995)).

Transformation of monocotyledons using electroporation, particle bombardment and Agrobacterium have also been reported. Transformation and plant regeneration have been achieved in asparagus (Bytebier et al., Proc. Natl. Acad. Sci. (USA) 84:5354 (1987)); barley (Wan and Lemaux, Plant Physiol 104:37 (1994)); maize (Rhodes et al., Science 240:204 (1988); Gordon-Kamm et al., Plant Cell 2:603-618 (1990); Fromm et al., Bio/Technology 8:833 (1990); Koziel et al., Bio/Technology 11:194 (1993); Armstrong et al., Crop Science 35:550-557 (1995)); oat (Somers et al., Bio/Technology 10:1589 (1992)); orchard grass (Horn et al., Plant Cell Rep. 7:469 (1988)); rice (Toriyama et al., Theor Appl. Genet. 205:34 (1986); Part et al., Plant Mol. Biol. 32:1135-1148 (1996); Abedinia et al., Aust. J. Plant Physiol. 24:133-141 (1997); Zhang and Wu, Theor. Appl. Genet. 76:835 (1988); Zhang et al., Plant Cell Rep. 7:379 (1988); Battraw and Hall, Plant Sci. 86:191-202 (1992); Christou et al., Bio/Technology 9:957 (1991)); rye (De la Pena et al., Nature 325:274 (1987)); sugarcane (Bower and Birch, Plant J. 2:409 (1992)); tall fescue (Wang et al., Bio/Technology 10:691 (1992)) and wheat (Vasil et al., Bio/Technology 10:667 (1992); U.S. Pat. No. 5,631,152).

Assays for gene expression based on the transient expression of cloned nucleic acid constructs have been developed by introducing the nucleic acid molecules into plant cells by polyethylene glycol treatment, electroporation, or particle bombardment (Marcotte et al., Nature 335:454-457 (1988); Marcotte et al., Plant Cell 1:523-532 (1989); McCarty et al., Cell 66:895-905 (1991); Hattori et al., Genes Dev. 6:609-618 (1992); Goff et al., EMBO J. 9:2517-2522 (1990)). Transient expression systems may be used to functionally dissect gene constructs (see generally, Mailga et al., Methods in Plant Molecular Biology, Cold Spring Harbor Press (1995)).

Any of the nucleic acid molecules of the invention may be introduced into a plant cell in a permanent or transient manner in combination with other genetic elements such as vectors, promoters, enhancers, etc. Further, any of the nucleic acid molecules of the invention may be introduced into a plant cell in a manner that allows for overexpression of the protein or fragment thereof encoded by the nucleic acid molecule.

Cosuppression is the reduction in expression levels, usually at the level of RNA, of a particular endogenous gene or gene family by the expression of a homologous sense construct that is capable of transcribing mRNA of the same strandedness as the transcript of the endogenous gene (Napoli et al., Plant Cell 2:279-289 (1990); van der Krol et al., Plant Cell 2:291-299 (1990)). Cosuppression may result from stable transformation with a single copy nucleic acid molecule that is homologous to a nucleic acid sequence found with the cell (Prolls and Meyer, Plant J. 2:465-475 (1992)) or with multiple copies of a nucleic acid molecule that is homologous to a nucleic acid sequence found with the cell (Mittlesten et al., Mol. Gen. Genet. 244:325-330 (1994)). Genes, even though different, linked to homologous promoters may result in the cosuppression of the linked genes (Vaucheret, C. R. Acad. Sci. III 316:1471-1483 (1993); Flavell, Proc. Natl. Acad. Sci. (U.S.A.) 91:3490-3496 (1994)); van Blokland et al., Plant J. 6:861-877 (1994); Jorgensen, Trends Biotechnol. 8:340-344 (1990); Meins and Kunz, In: Gene Inactivation and Homologous Recombination in Plants, Paszkowski (ed.), pp. 335-348, Kluwer Academic, Netherlands (1994)).

It is understood that one or more of the nucleic acids of the invention may be introduced into a plant cell and transcribed using an appropriate promoter with such transcription resulting in the cosuppression of an endogenous protein.

Antisense approaches are a way of preventing or reducing gene function by targeting the genetic material (Mol et al., FEBS Lett. 268:427-430 (1990)). The objective of the antisense approach is to use a sequence complementary to the target gene to block its expression and create a mutant cell line or organism in which the level of a single chosen protein is selectively reduced or abolished. Antisense techniques have several advantages over other ‘reverse genetic’ approaches. The site of inactivation and its developmental effect can be manipulated by the choice of promoter for antisense genes or by the timing of external application or microinjection. Antisense can manipulate its specificity by selecting either unique regions of the target gene or regions where it shares homology to other related genes (Hiatt et al., In: Genetic Engineering, Setlow (ed.), Vol. 11, New York: Plenum 49-63 (1989)).

The principle of regulation by antisense RNA is that RNA that is complementary to the target mRNA is introduced into cells, resulting in specific RNA:RNA duplexes being formed by base pairing between the antisense substrate and the target mRNA (Green et al., Annu. Rev. Biochem. 55:569-597 (1986)). Under one embodiment, the process involves the introduction and expression of an antisense gene sequence. Such a sequence is one in which part or all of the normal gene sequences are placed under a promoter in inverted orientation so that the ‘wrong’ or complementary strand is transcribed into a noncoding antisense RNA that hybridizes with the target mRNA and interferes with its expression (Takayama and Inouye, Crit. Rev. Biochem. Mol. Biol. 25:155-184 (1990)). An antisense vector is constructed by standard procedures and introduced into cells by transformation, transfection, electroporation, microinjection, infection, etc. The type of transformation and choice of vector will determine whether expression is transient or stable. The promoter used for the antisense gene may influence the level, timing, tissue, specificity, or inducibility of the antisense inhibition.

It is understood that the activity of a protein in a plant cell may be reduced or depressed by growing a transformed plant cell containing a nucleic acid molecule whose non-transcribed strand encodes a protein or fragment thereof.

Posttranscriptional gene silencing (PTGS) can result in virus immunity or gene silencing in plants. PTGS is induced by dsRNA and is mediated by an RNA-dependent RNA polymerase, present in the cytoplasm, that requires a dsRNA template. The dsRNA is formed by hybridization of complementary transgene mRNAs or complementary regions of the same transcript. Duplex formation can be accomplished by using transcripts from one sense gene and one antisense gene colocated in the plant genome, a single transcript that has self-complementarity, or sense and antisense transcripts from genes brought together by crossing. The dsRNA-dependent RNA polymerase makes a complementary strand from the transgene mRNA and RNAse molecules attach to this complementary strand (cRNA). These cRNA-RNAse molecules hybridize to the endogene mRNA and cleave the single-stranded RNA adjacent to the hybrid. The cleaved single-stranded RNAs are further degraded by other host RNAses because one will lack a capped 5′ end and the other will lack a poly(A) tail (Waterhouse et al., PNAS 95: 13959-13964 (1998)).

It is understood that one or more of the nucleic acids of the invention may be introduced into a plant cell and transcribed using an appropriate promoter with such transcription resulting in the postranscriptional gene silencing of an endogenous transcript.

Antibodies have been expressed in plants (Hiatt et al., Nature 342:76-78 (1989); Conrad and Fielder, Plant Mol. Biol. 26:1023-1030 (1994)). Cytoplasmic expression of a scFv (single-chain Fv antibodies) has been reported to delay infection by artichoke mottled crinkle virus. Transgenic plants that express antibodies directed against endogenous proteins may exhibit a physiological effect (Philips et al., EMBO J. 16:4489-4496 (1997); Marion-Poll, Trends in Plant Science 2:447-448 (1997)). For example, expressed anti-abscissic antibodies have been reported to result in a general perturbation of seed development (Philips et al., EMBO J. 16: 4489-4496 (1997)).

Antibodies that are catalytic may also be expressed in plants (abzymes). The principle behind abzymes is that since antibodies may be raised against many molecules, this recognition ability can be directed toward generating antibodies that bind transition states to force a chemical reaction forward (Persidas, Nature Biotechnology 15:1313-1315 (1997); Baca et al., Ann. Rev. Biophys. Biomol. Struct. 26:461-493 (1997)). The catalytic abilities of abzymes may be enhanced by site directed mutagenesis. Examples of abzymes are, for example, set forth in U.S. Pat. No. 5,658,753; U.S. Pat. No. 5,632,990; U.S. Pat. No. 5,631,137; U.S. Pat. No. 5,602,015; U.S. Pat. No. 5,559,538; U.S. Pat. No. 5,576,174; U.S. Pat. No. 5,500,358; U.S. Pat. No. 5,318,897; U.S. Pat. No. 5,298,409; U.S. Pat. No. 5,258,289 and U.S. Pat. No. 5,194,585.

It is understood that any of the antibodies of the invention may be expressed in plants and that such expression can result in a physiological effect. It is also understood that any of the expressed antibodies may be catalytic.

The present invention also provides for parts of the plants of the present invention. Plant parts, without limitation, include seed, endosperm, ovule and pollen. In a particularly preferred embodiment of the present invention, the plant part is a seed.

### Exemplary Uses

Nucleic acid molecules and fragments thereof of the invention may be employed to obtain other nucleic acid molecules from the same species (nucleic acid molecules from maize may be utilized to obtain other nucleic acid molecules from maize). Such nucleic acid molecules include the nucleic acid molecules that encode the complete coding sequence of a protein and promoters and flanking sequences of such molecules. In addition, such nucleic acid molecules include nucleic acid molecules that encode for other isozymes or gene family members. Such molecules can be readily obtained by using the above-described nucleic acid molecules or fragments thereof to screen cDNA or genomic libraries. Methods for forming such libraries are well known in the art.

Nucleic acid molecules and fragments thereof of the invention may also be employed to obtain nucleic acid homologues. Such homologues include the nucleic acid molecule of other plants or other organisms (e.g., alfalfa, Arabidopsis, barley, Brassica, broccoli, cabbage, citrus, cotton, garlic, oat, oilseed rape, onion, canola, flax, an ornamental plant, pea, peanut, pepper, potato, rice, lye, sorghum, strawberry, sugarcane, sugarbeet, tomato, wheat, poplar, pine, fir, eucalyptus, apple, lettuce, lentils, grape, banana, tea, turf grasses, sunflower, oil palm, Phaseolus, etc.) including the nucleic acid molecules that encode, in whole or in part, protein homologues of other plant species or other organisms, sequences of genetic elements, such as promoters and transcriptional regulatory elements. Such molecules can be readily obtained by using the above-described nucleic acid molecules or fragments thereof to screen cDNA or genomic libraries obtained from such plant species. Methods for forming such libraries are well known in the art. Such homologue molecules may differ in their nucleotide sequences from those found in one or more of SEQ ID NO: 1 through SEQ ID NO: 43 or complements thereof because complete complementarity is not needed for stable hybridization. The nucleic acid molecules of the invention therefore also include molecules that, although capable of specifically hybridizing with the nucleic acid molecules, may lack “complete complementarity.”

Any of a variety of methods may be used to obtain one or more of the above-described nucleic acid molecules (Zamechik et al., Proc. Natl. Acad. Sci. (U.S.A.) 83:4143-4146 (1986); Goodchild et al., Proc. Natl. Acad. Sci. (U.S.A.) 85:5507-5511 (1988); Wickstrom et al., Proc. Natl. Acad. Sci. (U.S.A.) 85:1028-1032 (1988); Holt et al., Molec. Cell. Biol. 8:963-973 (1988); Gerwirtz et al., Science 242:1303-1306 (1988); Anfossi et al., Proc. Natl. Acad. Sci. (U.S.A.) 86:3379-3383 (1989); Becker et al., EMBO J. 8:3685-3691 (1989)). Automated nucleic acid synthesizers may be employed for this purpose. In lieu of such synthesis, the disclosed nucleic acid molecules may be used to define a pair of primers that can be used with the polymerase chain reaction (Mullis et al., Cold Spring Harbor Symp. Quant. Biol. 51:263-273 (1986); Erlich et al., European Patent 50,424; European Patent 84,796; European Patent 258,017; European Patent 237,362; Mullis, European Patent 201,184; Mullis et al., U.S. Pat. No. 4,683,202; Erlich, U.S. Pat. No. 4,582,788; and Saiki et al., U.S. Pat. No. 4,683,194) to amplify and obtain any desired nucleic acid molecule or fragment.

Promoter sequences and other genetic elements, including but not limited to transcriptional regulatory flanking sequences, associated with one or more of the disclosed nucleic acid sequences can also be obtained using the disclosed nucleic acid sequence provided herein. In one embodiment, such sequences are obtained by incubating nucleic acid molecules of the present invention with members of genomic libraries and recovering clones that hybridize to such nucleic acid molecules thereof. In a second embodiment, methods of “chromosome walking,” or inverse PCR may be used to obtain such sequences (Frohman et al., Proc. Natl. Acad. Sci. (U.S.A.) 85:8998-9002 (1988); Ohara et al., Proc. Natl. Acad. Sci. (U.S.A.) 86:5673-5677 (1989); Pang et al., Biotechniques 22:1046-1048 (1977); Huang et al., Methods Mol. Biol. 69:89-96 (1997); Huang et al., Method Mol. Biol. 67:287-294 (1997); Benkel et al., Genet. Anal. 13:123-127 (1996); Hartl et al., Methods Mol. Biol. 58:293-301 (1996)). The term “chromosome walking” means a process of extending a genetic map by successive hybridization steps.

The nucleic acid molecules of the invention may be used to isolate promoters of cell enhanced, cell specific, tissue enhanced, tissue specific, developmentally or environmentally regulated expression profiles. Isolation and functional analysis of the 5′ flanking promoter sequences of these genes from genomic libraries, for example, using genomic screening methods and PCR techniques would result in the isolation of useful promoters and transcriptional regulatory elements. These methods are known to those of skill in the art and have been described (See, for example, Birren et al., Genome Analysis: Analyzing DNA, 1, (1997), Cold Spring Harbor Laboratory Press, Cold Spring Harbor, N.Y.). Promoters obtained utilizing the nucleic acid molecules of the invention could also be modified to affect their control characteristics. Examples of such modifications would include but are not limited to enhancer sequences. Such genetic elements could be used to enhance gene expression of new and existing traits for crop improvement.

Another subset of the nucleic acid molecules of the invention includes nucleic acid molecules that are markers. The markers can be used in a number of conventional ways in the field of molecular genetics. Such markers include nucleic acid molecules SEQ ID NO: 1 through SEQ ID NO: 43 or complements thereof or fragments of either that can act as markers and other nucleic acid molecules of the present invention that can act as markers.

Genetic markers of the invention include “dominant” or “codominant” markers. “Codominant markers” reveal the presence of two or more alleles (two per diploid individual) at a locus. “Dominant markers” reveal the presence of only a single allele per locus. The presence of the dominant marker phenotype (e.g., a band of DNA) is an indication that one allele is in either the homozygous or heterozygous condition. The absence of the dominant marker phenotype (e.g., absence of a DNA band) is merely evidence that “some other” undefined allele is present. In the case of populations where individuals are predominantly homozygous and loci are predominately dimorphic, dominant and codominant markers can be equally valuable. As populations become more heterozygous and multi-allelic, codominant markers often become more informative of the genotype than dominant markers. Marker molecules can be, for example, capable of detecting polymorphisms such as single nucleotide polymorphisms (SNPs).

SNPs can be characterized using any of a variety of methods (Botstein et al., Am. J. Hum. Genet. 32:314-331 (1980); Konieczny and Ausubel, Plant J. 4:403-410 (1993); Myers et al., Nature 313:495-498 (1985); Newton et al., Nucl. Acids Res. 17:2503-2516 (1989); Wu et al., Proc. Natl. Acad. Sci. (U.S.A.) 86:2757-2760 (1989); Barany, Proc. Natl. Acad. Sci. (U.S.A.) 88:189-193 (1991); Labrune et al., Am. J. Hum. Genet. 48: 1115-1120 (1991); Kuppuswami et al., Proc. Natl. Acad. Sci. USA 88:1143-1147 (1991); Sarkar et al., Genomics 13:441-443 (1992); Nikiforov et al., Nucl. Acids Res. 22:4167-4175 (1994); Livak et al., PCR Methods Appl. 4:357-362 (1995); Livak et al., Nature Genet. 9:341-342 (1995); Chen and Kwok, Nucl. Acids Res. 25:347-353 (1997); Tyagi et al., Nature Biotech. 16: 49-53 (1998); Haff and Smirnov, Genome Res. 7: 378-388 (1997); Neff et al., Plant J. 14:387-392 (1998)).

Additional markers, such as AFLP markers, RFLP markers and RAPD markers, can be utilized (Walton, Seed World 22-29 (July 1993); Burow and Blake, Molecular Dissection of Complex Traits, 13-29, Paterson (ed.), CRC Press, New York (1988)). Another marker type, RAPDs, is developed from DNA amplification with random primers and result from single base changes and insertions/deletions in plant genomes. They are dominant markers with a medium level of polymorphisms and are highly abundant. AFLP markers require using the PCR on a subset of restriction fragments from extended adapter primers. These markers are both dominant and codominant are highly abundant in genomes and exhibit a medium level of polymorphism.

The genomes of animals and plants naturally undergo spontaneous mutation in the course of their continuing evolution (Gusella, Ann. Rev. Biochem. 55:831-854 (1986)). A “polymorphism” is a variation or difference in the sequence of the gene or its flanking regions that arises in some of the members of a species. The variant sequence and the “original” sequence co-exist in the species' population. In some instances, such co-existence is in stable or quasi-stable equilibrium.

A polymorphism is thus said to be “allelic,” in that, due to the existence of the polymorphism, some members of a species may have the original sequence (i.e., the original “allele”) whereas other members may have the variant sequence (i.e., the variant “allele”). In the simplest case, only one variant sequence may exist and the polymorphism is thus said to be di-allelic. In other cases, the species' population may contain multiple alleles and the polymorphism is termed tri-allelic, etc. A single gene may have multiple different unrelated polymorphisms. For example, it may have a di-allelic polymorphism at one site and a multi-allelic polymorphism at another site.

The variation that defines the polymorphism may range from a single nucleotide variation to the insertion or deletion of extended regions within a gene. In some cases, the DNA sequence variations are in regions of the genome that are characterized by short tandem repeats (STRs) that include tandem di- or tri-nucleotide repeated motifs of nucleotides. Polymorphisms characterized by such tandem repeats are referred to as “variable number tandem repeat” (“VNTR”) polymorphisms. VNTRs have been used in identity analysis (Weber, U.S. Pat. No. 5,075,217; Armour et al., FEBS Lett. 307:113-115 (1992); Jones et al., Eur. J. Haematol. 39:144-147 (1987); Horn et al., PCT Patent Application WO91/14003; Jeffreys, European Patent Application 370,719; Jeffreys, U.S. Pat. No. 5,175,082; Jeffreys et al., Amer. J. Hum. Genet. 39:11-24 (1986); Jeffreys et al., Nature 316:76-79 (1985); Gray et al., Proc. R. Acad. Soc. Lond. 243:241-253 (1991); Moore et al., Genomics 10:654-660 (1991); Jeffreys et al., Anim. Genet. 18:1-15 (1987); Hillel et al., Anim. Genet. 20:145-155 (1989); Hillel et al., Genet. 124:783-789 (1990)).

The detection of polymorphic sites in a sample of DNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis or other means.

In an alternative embodiment, such polymorphisms can be detected through the use of a marker nucleic acid molecule that is physically linked to such polymorphism(s). For this purpose, marker nucleic acid molecules comprising a nucleotide sequence of a polynucleotide located within 1 mb of the polymorphism(s) and more preferably within 100 kb of the polymorphism(s) and most preferably within 10 kb of the polymorphism(s) can be employed.

The identification of a polymorphism can be determined in a variety of ways. By correlating the presence or absence of it in a plant with the presence or absence of a phenotype, it is possible to predict the phenotype of that plant. If a polymorphism creates or destroys a restriction endonuclease cleavage site, or if it results in the loss or insertion of DNA (e.g., a VNTR polymorphism), it will alter the size or profile of the DNA fragments that are generated by digestion with that restriction endonuclease. As such, individuals that possess a variant sequence can be distinguished from those having the original sequence by restriction fragment analysis. Polymorphisms that can be identified in this manner are termed “restriction fragment length polymorphisms” (“RFLPs”) (Glassberg, UK Patent Application 2135774; Skolnick et al., Cytogen. Cell Genet. 32:58-67 (1982); Botstein et al., Ann. J. Hum. Genet. 32:314-331 (1980); Fischer et al., (PCT Application WO90/13668; Uhlen, PCT Application WO90/11369).

Polymorphisms can also be identified by Single Strand Conformation Polymorphism (SSCP) analysis (Elles, Methods in Molecular Medicine: Molecular Diagnosis of Genetic Diseases, Humana Press (1996)); Orita et al., Genomics 5:874-879 (1989)). A number of protocols have been described for SSCP including, but not limited to, Lee et al., Anal. Biochem. 205:289-293 (1992); Suzuki et al., Anal. Biochem. 192:82-84 (1991); Lo et al., Nucleic Acids Research 20:1005-1009 (1992); Sarkar et al., Genomics 13:441-443 (1992). It is understood that one or more of the nucleic acids of the invention may be utilized as markers or probes to detect polymorphisms by SSCP analysis.

Polymorphisms may also be found using a DNA fingerprinting technique called amplified fragment length polymorphism (AFLP), which is based on the selective PCR amplification of restriction fragments from a total digest of genomic DNA to profile that DNA (Vos et al., Nucleic Acids Res. 23:4407-4414 (1995)). This method allows for the specific co-amplification of high numbers of restriction fragments, which can be visualized by PCR without knowledge of the nucleic acid sequence. It is understood that one or more of the nucleic acids of the invention may be utilized as markers or probes to detect polymorphisms by AFLP analysis or for fingerprinting RNA.

Polymorphisms may also be found using random amplified polymorphic DNA (RAPD) (Williams et al., Nucl. Acids Res. 18:6531-6535 (1990)) and cleaveable amplified polymorphic sequences (CAPS) (Lyamichev et al., Science 260:778-783 (1993)). It is understood that one or more of the nucleic acid molecules of the invention may be utilized as markers or probes to detect polymorphisms by RAPD or CAPS analysis.

Through genetic mapping, a fine scale linkage map can be developed using DNA markers and, then, a genomic DNA library of large-sized fragments can be screened with molecular markers linked to the desired trait. Molecular markers are advantageous for agronomic traits that are otherwise difficult to tag, such as resistance to pathogens, insects and nematodes, tolerance to abiotic stress, quality parameters and quantitative traits such as high yield potential. Here, an altered phytosterol level is a preferred trait.

Essential requirements for marker-assisted selection in a plant breeding program are: (1) the marker(s) should co-segregate or be closely linked with the desired trait; (2) an efficient means of screening large populations for the molecular marker(s) should be available; and (3) the screening technique should have high reproducibility across laboratories and preferably be economical to use and be user-friendly.

The genetic linkage of marker molecules can be established by a gene mapping model such as, without limitation, the flanking marker model reported by Lander and Botstein, Genetics 121:185-199 (1989) and the interval mapping, based on maximum likelihood methods described by Lander and Botstein, Genetics 121:185-199 (1989) and implemented in the software package MAPMAKER/QTL (Lincoln and Lander, Mapping Genes Controlling Quantitative Traits Using MAPMAKER/QTL, Whitehead Institute for Biomedical Research, Massachusetts, (1990). Additional software includes Qgene, Version 2.23 (1996), Department of Plant Breeding and Biometry, 266 Emerson Hall, Cornell University, Ithaca, N.Y.). Use of Qgene software is a particularly preferred approach.

A maximum likelihood estimate (MLE) for the presence of a marker is calculated, together with an MLE assuming no QTL effect, to avoid false positives. A log₁₀ of an odds ratio (LOD) is then calculated as: LOD=log₁₀ (MLE for the presence of a QTL/MLE given no linked QTL).

The LOD score essentially indicates how much more likely the data are to have arisen assuming the presence of a QTL than in its absence. The LOD threshold value for avoiding a false positive with a given confidence, say 95%, depends on the number of markers and the length of the genome. Graphs indicating LOD thresholds are set forth in Lander and Botstein, Genetics 121:185-199 (1989) and further described by Arús and Moreno-González, Plant Breeding, Hayward et al., (eds.) Chapman & Hall, London, pp. 314-331 (1993).

Additional models can be used. Many modifications and alternative approaches to interval mapping have been reported, including the use non-parametric methods (Kruglyak and Lander, Genetics 139:1421-1428 (1995)). Multiple regression methods or models can be also be used, in which the trait is regressed on a large number of markers (Jansen, Biometrics in Plant Breeding, van Oijen and Jansen (eds.), Proceedings of the Ninth Meeting of the Eucarpia Section Biometrics in Plant Breeding, The Netherlands, pp. 116-124 (1994); Weber and Wricke, Advances in Plant Breeding, Blackwell, Berlin, 16 (1994)). Procedures combining interval mapping with regression analysis, whereby the phenotype is regressed onto a single putative QTL at a given marker interval and at the same time onto a number of markers that serve as ‘cofactors,’ have been reported by Jansen and Stam, Genetics 136:1447-1455 (1994), and Zeng, Genetics 136:1457-1468 (1994). Generally, the use of cofactors reduces the bias and sampling error of the estimated QTL positions (Utz and Melchinger, Biometrics in Plant Breeding, van Oijen and Jansen (eds.) Proceedings of the Ninth Meeting of the Eucarpia Section Biometrics in Plant Breeding, The Netherlands, pp. 195-204 (1994), thereby improving the precision and efficiency of QTL mapping (Zeng, Genetics 136:1457-1468 (1994)). These models can be extended to multi-environment experiments to analyze genotype-environment interactions (Jansen et al., Theo. Appl. Genet. 91:33-37 (1995)).

It is understood that one or more of the nucleic acid molecules of the invention may be used as molecular markers. It is also understood that one or more of the protein molecules of the invention may be used as molecular markers.

In accordance with this aspect of the invention, a sample nucleic acid is obtained from plant cells or tissues. Any source of nucleic acid may be used. Preferably, the nucleic acid is genomic DNA. The nucleic acid is subjected to restriction endonuclease digestion. For example, one or more nucleic acid molecule or fragment thereof of the invention can be used as a probe in accordance with the above-described polymorphic methods. The polymorphism obtained in this approach can then be cloned to identify the mutation at the coding region, which alters structure, or regulatory region of the gene, which affects its expression level.

In an aspect of the present invention, one or more of the nucleic molecules of the present invention are used to determine the level (i.e., the concentration of mRNA in a sample, etc.) in a plant (preferably maize or soybean) or pattern (i.e., the kinetics of expression, rate of decomposition, stability profile, etc.) of the expression of a protein encoded in part or whole by one or more of the nucleic acid molecule of the present invention (collectively, the “Expression Response” of a cell or tissue).

As used herein, the Expression Response manifested by a cell or tissue is said to be “altered” if it differs from the Expression Response of cells or tissues of plants not exhibiting the phenotype. To determine whether an Expression Response is altered, the Expression Response manifested by the cell or tissue of the plant exhibiting the phenotype is compared with that of a similar cell or tissue sample of a plant not exhibiting the phenotype. As will be appreciated, it is not necessary to re-determine the Expression Response of the cell or tissue sample of plants not exhibiting the phenotype each time such a comparison is made; rather, the Expression Response of a particular plant may be compared with previously obtained values of normal plants. As used herein, the phenotype of the organism is any of one or more characteristics of an organism (e.g. disease resistance, pest tolerance, environmental tolerance such as tolerance to abiotic stress, male sterility, quality improvement or yield etc.). A change in genotype or phenotype may be transient or permanent. Also as used herein, a tissue sample is any sample that comprises more than one cell. In a preferred aspect, a tissue sample comprises cells that share a common characteristic (e.g. derived from root, seed, flower, leaf, stem or pollen etc.).

In one aspect of the present invention, an evaluation can be conducted to determine whether a particular mRNA molecule is present. One or more of the nucleic acid molecules of the present invention, preferably one or more of the nucleic acid molecules of the present invention are utilized to detect the presence or quantity of the mRNA species. Such molecules are then incubated with cell or tissue extracts of a plant under conditions sufficient to permit nucleic acid hybridization. The detection of double-stranded probe-mRNA hybrid molecules is indicative of the presence of the mRNA; the amount of such hybrid formed is proportional to the amount of mRNA. Thus, such probes may be used to ascertain the level and extent of the mRNA production in a plant's cells or tissues. Such nucleic acid hybridization may be conducted under quantitative conditions (thereby providing a numerical value of the amount of the mRNA present). Alternatively, the assay may be conducted as a qualitative assay that indicates either that the mRNA is present, or that its level exceeds a user set, predefined value.

A number of methods can be used to compare the expression response between two or more samples of cells or tissue. These methods include hybridization assays, such as Northerns, RNAse protection assays, and in situ hybridization. Alternatively, the methods include PCR-type assays. In a preferred method, the expression response is compared by hybridizing nucleic acids from the two or more samples to an array of nucleic acids. The array contains a plurality of suspected sequences known or suspected of being present in the cells or tissue of the samples.

An advantage of in situ hybridization over more conventional techniques for the detection of nucleic acids is that it allows an investigator to determine the precise spatial population (Angerer et al., Dev. Biol. 101:477-484 (1984); Angerer et al., Dev. Biol. 112:157-166 (1985); Dixon et al., EMBO J. 10:1317-1324 (1991)). In situ hybridization may be used to measure the steady-state level of RNA accumulation (Hardin et al., J. Mol. Biol. 202:417-431 (1989)). A number of protocols have been devised for in situ hybridization, each with tissue preparation, hybridization and washing conditions (Meyerowitz, Plant Mol. Biol. Rep. 5:242-250 (1987); Cox and Goldberg, In: Plant Molecular Biology: A Practical Approach, Shaw (ed.), pp. 1-35, IRL Press, Oxford (1988); Raikhel et al., In situ RNA hybridization in plant tissues, In: Plant Molecular Biology Manual, vol. B9:1-32, Kluwer Academic Publisher, Dordrecht, Belgium (1989)).

In situ hybridization also allows for the localization of proteins within a tissue or cell (Wilkinson, In Situ Hybridization, Oxford University Press, Oxford (1992); Langdale, In Situ Hybridization In: The Maize Handbook, Freeling and Walbot (eds.), pp. 165-179, Springer-Verlag, New York (1994)). It is understood that one or more of the molecules of the invention, preferably one or more of the nucleic acid molecules or fragments thereof of the invention or one or more of the antibodies of the invention may be utilized to detect the level or pattern of a protein or mRNA thereof by in situ hybridization.

Fluorescent in situ hybridization allows the localization of a particular DNA sequence along a chromosome which is useful, among other uses, for gene mapping, following chromosomes in hybrid lines or detecting chromosomes with translocations, transversions or deletions. In situ hybridization has been used to identify chromosomes in several plant species (Griffor et al., Plant Mol. Biol. 17:101-109 (1991); Gustafson et al., Proc. Natl. Acad. Sci. (U.S.A.) 87:1899-1902 (1990); Mukai and Gill, Genome 34:448-452 (1991); Schwarzacher and Heslop-Harrison, Genome 34:317-323 (1991); Wang et al., Jpn. J. Genet. 66:313-316 (1991); Parra and Windle, Nature Genetics 5:17-21 (1993)). It is understood that the nucleic acid molecules of the invention may be used as probes or markers to localize sequences along a chromosome.

Another method to localize the expression of a molecule is tissue printing. Tissue printing provides a way to screen, at the same time on the same membrane many tissue sections from different plants or different developmental stages (Yomo and Taylor, Planta 112:35-43 (1973); Harris and Chrispeels, Plant Physiol. 56:292-299 (1975); Cassab and Varner, J. Cell. Biol. 105:2581-2588 (1987); Spruce et al., Phytochemistry 26:2901-2903 (1987); Barres et al., Neuron 5:527-544 (1990); Reid and Pont-Lezica, Tissue Printing Tools for the Study of Anatomy, Histochemistry and Gene Expression, Academic Press, New York, N.Y. (1992); Reid et al., Plant Physiol. 93:160-165 (1990); Ye et al., Plant J. 1:175-183 (1991)).

It is understood that one or more of the molecules of the invention, preferably one or more of the nucleic acid molecules of the present invention or one or more of the antibodies of the invention may be utilized to detect the presence or quantity of a protein or fragment of the invention by tissue printing.

Further it is also understood that any of the nucleic acid molecules of the invention may be used as marker nucleic acids and or probes in connection with methods that require probes or marker nucleic acids. As used herein, a probe is an agent that is utilized to determine an attribute or feature (e.g. presence or absence, location, correlation, etc.) of a molecule, cell, tissue or plant. As used herein, a marker nucleic acid is a nucleic acid molecule that is utilized to determine an attribute or feature (e.g., presence or absence, location, correlation, etc.) or a molecule, cell, tissue or plant.

A microarray-based method for high-throughput monitoring of gene expression may be utilized to measure expression response Schena et al., Science 270:467-470 (1995); on the website cmgm.stanford.edu/pbrown/array.html; Shalon, Ph.D. Thesis, Stanford University (1996). This approach is based on using arrays of DNA targets (e.g. cDNA inserts, colonies, or polymerase chain reaction products) for hybridization to a “complex probe” prepared with RNA extracted from a given cell line or tissue. The probe may be produced by reverse transcription of mRNA or total RNA and labeled with radioactive or fluorescent labeling. The probe is complex in that it contains many different sequences in various amounts, corresponding to the numbers of copies of the original mRNA species extracted from the sample.

The initial RNA source will typically be derived from a physiological source. The physiological source may be derived from a variety of eukaryotic sources, with physiological sources of interest including sources derived from single celled organisms such as yeast and multicellular organisms, including plants and animals, particularly plants, where the physiological sources from multicellular organisms may be derived from particular organs or tissues of the multicellular organism, or from isolated cells derived therefrom. The physiological sources may be derived from multicellular organisms at different developmental stages (e.g., 10-day-old seedlings), grown under different environmental conditions (e.g., drought-stressed plants) or treated with chemicals.

In obtaining the sample of RNAs to be analyzed from the physiological source from which it is derived, the physiological source may be subjected to a number of different processing steps, where such processing steps might include tissue homogenation, cell isolation and cytoplasmic extraction, nucleic acid extraction and the like, where such processing steps are known to the those of skill in the art. Methods of isolating RNA from cells, tissues, organs or whole organisms are known to those of skill in the art and are described in Maniatis et al., Molecular Cloning: A Laboratory Manual (Cold Spring Harbor Press) (1989).

The DNA may be placed on nylon or glass “microarrays” regularly arranged with a spot spacing of 1 mm or less. Expression levels can be measured for hundreds or thousands of genes, by using less than 2 micrograms of polyA+ RNA and determining the relative mRNA abundances down to one in ten thousand or less (Granjeaud et al., BioEssays 21:781-790 (1999)).

In addition to arrays of cDNA clones or inserts, arrays of oligonucleotides are also used to study differential gene expression. In an oligonucleotide array, the genes of interest are represented by a series of approximately 20 nucleotide oligomers that are unique to each gene. Labeled mRNA is prepared and hybridization signals are detected from specific sets of oligos that represent different genes supplemented by a set of control oligonucleotides. Potential advantages of the oligonucleotide array include enhanced specificity and sensitivity through the parallel analysis of “perfect match” oligos and “mismatch” oligos for each gene. The hybridization conditions can be adjusted to distinguish a perfect heteroduplex from a single base mismatch, thus allowing subtraction of nonspecific hybridization signals from specific hybridization signals. A disadvantage of oligonucleotide arrays relative to cDNA arrays is the limitation of the technology to genes of known sequence (Granjeaud et al., BioEssays 21:781-790 (1991); Carulli et al., Journal of Cellular Biochemistry Supplements 30/31:286-296 (1998)).

These techniques have been successfully used to characterize patterns of gene expression associated with, for example, various important physiological changes in yeast, including the mitotic cell cycle, the heat shock response, and comparison between mating types. Once a set of comparable expression profiles is obtained, e.g. for cells at different time points or at different cellular states, a clustering algorithm generally is used to group sets of genes which share similar expression patterns. The clusters obtained can then be analyzed in the light of available functional annotations, often leading to associations of poorly characterized genes with genes whose function and regulation are better understood.

Regulatory networks that control gene expression can be characterized using microarray technology (DeRisi et al., Science 278: 680-686 (1997); Winzler et al. Science 28: 1194-1197 (1998); Cho et al. Mol Cell 2: 65-73 (1998); Spellman et al. Mol Biol Cell 95: 14863-14868 (1998). For example, it is has been reported that both cDNA and oligonucleotide arrays have been used to monitor gene expression in synchronized cell cultures. Analysis of the corresponding temporal patterns of gene expression resulted in the identification of over 400 cell cycle-regulated genes. In order to identify possible common regulatory mechanisms accounting for co-expression, consensus motifs in putative regulatory sequences upstream of the corresponding ORFs were examined. This resulted in the identification of several new potential binding sites for known factors or complexes involved in the coordinated transcription of genes during specific phases of the cell cycle (Thieffry, D. BioEssays 21: 895-899 (1999)).

The microarray approach may be used with polypeptide targets (U.S. Pat. No. 5,445,934; U.S. Pat. No. 5,143,854; U.S. Pat. No. 5,079,600; U.S. Pat. No. 4,923,901) synthesized on a substrate (microarray) and these polypeptides can be screened with either (Fodor et al., Science 251:767-773 (1991)). It is understood that one or more of the nucleic acid molecules or protein or fragments thereof of the invention may be utilized in a microarray-based method.

In another even more preferred embodiment of the present invention microarrays may be prepared that comprise nucleic acid molecules where such nucleic acid molecules include at least one, preferably at least two, more preferably at least three, even more preferably at least five, ten, fifteen, twenty, twenty-five, thirty, or thirty-five or more nucleic acid molecules or fragments thereof comprising a nucleic acid molecule selected from the group consisting of SEQ ID NO: 1 through SEQ ID NO: 43.

In another even more preferred embodiment of the present invention microarrays may be prepared that comprise nucleic acid molecules where such nucleic acid molecules include at least one, preferably at least two, more preferably at least three, even more preferably at least five, ten, fifteen, twenty, twenty-five, thirty, or thirty-five or more nucleic acid molecules or fragments thereof which specifically hybridize one or more nucleic acid molecules set forth in SEQ ID NO: 1 through SEQ ID NO: 43.

In yet another even more preferred embodiment of the present invention microarrays may be prepared that comprise nucleic acid molecules where such nucleic acid molecules encode at least one, preferably at least two, more preferably at least three, even more preferably at least five, ten, fifteen, twenty, twenty-five, thirty, or thirty-five or more proteins or fragment thereof comprising an amino acid sequence selected from the group consisting of SEQ ID NO: 44 through SEQ ID NO: 86.

Site directed mutagenesis may be utilized to modify nucleic acid sequences, particularly as it is a technique that allows one or more of the amino acids encoded by a nucleic acid molecule to be altered (e.g., a threonine to be replaced by a methionine) (Wells et al., Gene 34:315-323 (1985); Gilliam et al., Gene 12:129-137 (1980); Zoller and Smith, Methods Enzymol. 100:468-500 (1983); Dalbadie-McFarland et al., Proc. Natl. Acad. Sci. (U.S.A.) 79:6409-6413 (1982); Scharf et al., Science 233:1076-1078 (1986); Higuchi et al., Nucleic Acids Res. 16:7351-7367 (1988); U.S. Pat. No. 5,811,238, European Patent 0 385 962; European Patent 0 359 472; and PCT Patent Application WO 93/07278; Lanz et al., J. Biol. Chem. 266:9971-9976 (1991); Kovgan and Zhdanov, Biotekhnologiya 5:148-154, No. 207160n, Chemical Abstracts 110:225 (1989); Ge et al., Proc. Natl. Acad. Sci. (U.S.A.) 86:4037-4041 (1989); Zhu et al., J. Biol. Chem. 271:18494-18498 (1996); Chu et al., Biochemistry 33:6150-6157 (1994); Small et al., EMBO J. 11:1291-1296 (1992); Cho et al., Mol. Biotechnol. 8:13-16 (1997); Kita et al., 1 Biol. Chem. 271:26529-26535 (1996); Jin et al., Mol. Microbiol. 7:555-562 (1993); Hatfield and Vierstra, J. Biol. Chem. 267:14799-14803 (1992); Zhao et al., Biochemistry 31:5093-5099 (1992)).

Any of the nucleic acid molecules of the invention may either be modified by site directed mutagenesis or used as, for example, nucleic acid molecules that are used to target other nucleic acid molecules for modification.

It is understood that mutants with more than one altered nucleotide can be constructed using techniques that practitioners are familiar with, such as isolating restriction fragments and ligating such fragments into an expression vector (see, for example, Sambrook et al., Molecular Cloning: A Laboratory Manual, Cold Spring Harbor Press (1989)).

Two steps may be employed to characterize DNA-protein interactions. The first is to identify sequence fragments that interact with DNA-binding proteins, to titrate binding activity, to determine the specificity of binding and to determine whether a given DNA-binding activity can interact with related DNA sequences (Sambrook et al., Molecular Cloning: A Laboratory Manual, 2′^{d} edition, Cold Spring Harbor Laboratory Press, Cold Spring Harbor, N.Y. (1989)). Electrophoretic mobility-shift assay is a widely used assay. The assay provides a rapid and sensitive method for detecting DNA-binding proteins based on the observation that the mobility of a DNA fragment through a nondenaturing, low-ionic strength polyacrylamide gel is retarded upon association with a DNA-binding protein (Fried and Crother, Nucleic Acids Res. 9:6505-6525 (1981)). When one or more specific binding activities have been identified, the exact sequence of the DNA bound by the protein may be determined.

Several procedures for characterizing protein/DNA-binding sites are used (Maxam and Gilbert, Methods Enzymol. 65:499-560 (1980); Wissman and Hillen, Methods Enzymol. 208:365-379 (1991); Galas and Schmitz, Nucleic Acids Res. 5:3157-3170 (1978); Sigman et al., Methods Enzymol. 208:414-433 (1991); Dixon et al., Methods Enzymol. 208:414-433 (1991)). It is understood that one or more of the nucleic acid molecules of the invention may be utilized to identify a protein or fragment thereof that specifically binds to a nucleic acid molecule of the invention. It is also understood that one or more of the protein molecules or fragments thereof of the invention may be utilized to identify a nucleic acid molecule that specifically binds to it.

A two-hybrid system is based on the fact that proteins, such as transcription factors that interact (physically) with one another carry out many cellular functions. Two-hybrid systems have been used to probe the function of new proteins (Chien et al., Proc. Natl. Acad. Sci. (U.S.A.) 88:9578-9582 (1991); Durfee et al., Genes Dev. 7:555-569 (1993); Choi et al., Cell 78:499-512 (1994); Kranz et al., Genes Dev. 8:313-327 (1994)).

Interaction mating techniques have facilitated a number of two-hybrid studies of protein-protein interaction. Interaction mating has been used to examine interactions between small sets of tens of proteins (Finley and Brent, Proc. Natl. Acad. Sci. (U.S.A.) 91:12098-12984 (1994)), larger sets of hundreds of proteins (Bendixen et al., Nucl. Acids Res. 22:1778-1779 (1994)) and to comprehensively map proteins encoded by a small genome (Bartel et al., Nature Genetics 12:72-77 (1996)). This technique utilizes proteins fused to the DNA-binding domain and proteins fused to the activation domain. They are expressed in two different haploid yeast strains of opposite mating type and the strains are mated to determine if the two proteins interact. Mating occurs when haploid yeast strains come into contact and result in the fusion of the two haploids into a diploid yeast strain. An interaction can be determined by the activation of a two-hybrid reporter gene in the diploid strain.

It is understood that the protein-protein interactions of protein or fragments thereof of the invention may be investigated using the two-hybrid system and that any of the nucleic acid molecules of the invention that encode such proteins or fragments thereof may be used to transform yeast in the two-hybrid system.

### Computer Readable Media

The nucleotide sequence provided in SEQ ID NO:1 through SEQ ID NO: 43 or fragment thereof, or complement thereof, or a nucleotide sequence at least 70% identical, preferably 90% identical even more preferably 99% or about 100% identical to one or more of the nucleic acid sequences provided in SEQ ID NO: 1 through SEQ ID NO: 43 or complement thereof or fragments of either, can be “provided” in a variety of mediums to facilitate use.

In a preferred embodiment, 2, preferably 5, more preferably 10, even more preferably 25, 35, 50, or 75 of nucleic acid or amino acid sequences of the present invention can be provided in a variety of mediums.

In another aspect, the nucleotide sequences which correspond to those that encode one or more of the amino acid sequence provided in SEQ ID NO:44 through SEQ ID NO: 86 or fragment thereof can be provided in a variety of mediums to facilitate use.

In another aspect, one or more of the amino acid sequence provided in SEQ ID NO: 44 through SEQ ID NO: 86 or fragment thereof, or an amino acid sequence at least 70% identical, preferably 90% identical even more preferably 99% or about 100% identical to the sequence provided in SEQ ID NO: 44 through SEQ ID NO: 86 or fragments thereof, can be provided in a variety of mediums to facilitate use.

Such a medium can also provide a subset thereof in a form that allows a skilled artisan to examine the sequences.

In one application of this embodiment, a nucleotide sequence of the invention can be recorded on computer readable media so that a computer-readable medium comprises one or more of the nucleotide sequences of the invention. As used herein, “computer readable media” refers to any medium that can be read and accessed directly by a computer. Such media include, but are not limited to: magnetic storage media, such as floppy discs, hard disc, storage medium and magnetic tape; optical storage media such as CD-ROM; electrical storage media such as RAM and ROM; and hybrids of these categories such as magnetic/optical storage media.

Any number of the sequences, or sequence fragments, of the nucleic acid molecules or proteins of the invention, or fragments of either, can be included, in any number of combinations, on a computer-readable medium.

By providing one or more of nucleotide sequences of the invention, a skilled artisan can routinely access the sequence information for a variety of purposes. Computer software is publicly available that allows a skilled artisan to access sequence information provided in a computer readable medium. The examples which follow demonstrate how software which implements the BLAST (Altschul et al., J. Mol. Biol. 215:403-410 (1990)) and BLAZE (Brutlag et al., Comp. Chem. 17:203-207 (1993)) search algorithms on a Sybase system can be used to identify open reading frames (ORFs) within the genome that contain homology to ORFs or proteins from other organisms.

The invention further provides systems, particularly computer-based systems, which contain the sequence information described herein. Such systems are designed to identify commercially important fragments of the nucleic acid molecule of the invention. As used herein, “a computer-based system” refers to the hardware means, software means and data storage means used to analyze the nucleotide sequence information of the invention. The minimum hardware means of the computer-based systems of the invention comprises a central processing unit (CPU), input means, output means and data storage means. A skilled artisan can readily appreciate that any one of the currently available computer-based systems is suitable for use in the invention.

A variety of comparing means can be used to compare a target sequence or target motif with the data storage means to identify sequence fragments sequence of the invention. For example, implementing software that implements the BLAST and BLAZE algorithms (Altschul et al., J. Mol. Biol. 215:403-410 (1990)) can be used to identify open frames within the nucleic acid molecules of the invention. A skilled artisan can readily recognize that any one of the publicly available homology search programs can be used as the search means for the computer-based systems of the invention.

Having now described the invention, the following examples are provided by way of illustration and are not intended to limit the scope of the invention, unless specified.

### Example 1

Nucleic acid sequences encoding proteins are identified from the NCBI nr.aa database searched with BLASTX (default values) using full length insert sequences as queries (see Table 1) with a cutoff parameter of 1e-8.

TABLE 1

| Seq | | | | BLAST | | | Qstart | (nt)Sstart |
|---|---|---|---|---|---|---|---|---|
| Num | Seq ID | Library | NCBI gi | score | E value | % Ident | Qend | Send(aa) |
| 1 | fC-zmflm017233c12 | LIB3205 | 6907085 | 85.0 | 4e−16 | 68 | 52-240 | 339-405 |
| 2 | fC-zmst1700335931 | NONE | 7228459 | 265 | 7e−70 | 92 | 993-1421 | 1-143 |
| 3 | fC-zmflb73182c08 | LIB3206 | 4006908 | 102 | 8e−21 | 26 | 16-1023 | 293-617 |
| 4 | fC-zmrob73058d05 | LIB3239 | 6322653 | 78.8 | 8e−14 | 38 | 165-521 | 261-388 |
| 5 | fC-zmflb73189c09 | LIB3206 | 6983875 | 83.9 | 2e−15 | 96 | 13-147 | 272-321 |
| 6 | fC-zmrob73050f12 | LIB3239 | 2980788 | 86.6 | 4e−16 | 40 | 17-646 | 186-422 |
| 7 | fC-gmse7000753078 | NONE | 3702326 | 331 | 9e−90 | 81 | 139-720 | 1-192 |
| 8 | fC-gmse7000757563 | SOYMON015 | 6682251 | 404 | 1e−112 | 75 | 140-931 | 1-266 |
| 9 | fC-zmro038e08 | NONE | 7320718 | 95.2 | 6e−19 | 49 | 11-322 | 1181-1283 |
| 10 | fC-gmse700752221 | SOYMON014 | 4455198 | 450 | 1e−125 | 55 | 34-1431 | 1-418 |
| 11 | fC-2mrob73075c08 | LIB3239 | 6561955 | 189 | 9e−47 | 66 | 248-676 | 1-147 |
| 12 | fC-zmrob73076b02 | LIB3239 | 6016691 | 460 | 1e−128 | 58 | 276-1358 | 93-451 |
| 13 | fC-zmro084f01 | NONE | 7269838 | 152 | 6e−36 | 70 | 15-329 | 185-288 |
| 14 | fC-gmst700890412 | SOYMON024 | 4417289 | 217 | 8e−56 | 61 | 14-598 | 1-201 |
| 15 | fC-zmrob73057h03 | LIB3239 | 6598671 | 204 | 1e−51 | 41 | 13-855 | 613-877 |
| 16 | fC-zmrob73078g06 | LIB3239 | 4678268 | 159 | 5e−38 | 75 | 685-969 | 514-609 |
| 17 | fC-gmle700742801 | SOYMON012 | 4539400 | 63.6 | 2e−09 | 47 | 169-399 | 469-544 |
| 18 | fC-zmflb73011f10 | LIB3206 | 6907089 | 115 | 3e−24 | 31 | 471-1247 | 57-311 |
| 19 | fC-gmse700756777 | SOYMON014 | 6692265 | 356 | 4e−97 | 59 | 223-1248 | 1-324 |
| 20 | fC-zmflb73129d04 | LIB3206 | 3204103 | 164 | 3e−39 | 64 | 1135-1524 | 1-129 |
| 21 | fC-zmflb73135d04 | LIB3206 | 6522552 | 96.7 | 3e−19 | 40 | 129-434 | 1118-1219 |
| 22 | fC-zmflmo17128f05 | LIB3205 | 6041800 | 124 | 2e−27 | 30 | 4-1683 | 311-902 |
| 23 | fC-zmflb73143h07 | LIB3206 | 6522547 | 83.1 | 4e−15 | 51 | 94-324 | 54-130 |
| 24 | fC-zmflb73125a02 | LIB3206 | 6016734 | 612 | 1e−174 | 74 | 12-1256 | 754-1186 |
| 25 | fC-zmflmo17133c04 | LIB3205 | 6175174 | 526 | 1e−148 | 74 | 25-1083 | 1340-1688 |
| 26 | fC-zmflb73210d02 | LIB3206 | 6143875 | 169 | 3e−41 | 60 | 12-419 | 1139-1275 |
| 27 | fC-zmflb73271d01 | LIB3206 | 6587865 | 62.5 | 5e−09 | 37 | 234-581 | 58-179 |
| 28 | fC-zmflmo17185d02 | LIB3205 | 4049346 | 87.8 | 2e−16 | 32 | 382-1149 | 70-370 |
| 29 | fC-zmflb73013h12 | NONE | 5042445 | 247 | 1e−64 | 70 | 349-990 | 519-764 |
| 30 | fC-zmflmo17255f07 | LIB3205 | 1001384 | 70.2 | 1e−11 | 44 | 165-509 | 118-247 |
| 31 | fC-zmflmo17019e07 | LIB3205 | 2618699 | 143 | 2e−33 | 48 | 22-393 | 355-478 |
| 32 | fC-zmflmo17137g11 | LIB3205 | 4914426 | 76.1 | 7e−13 | 28 | 51-719 | 110-318 |
| 33 | fC-zmrob73002h02 | LIB3239 | 6539560 | 1342 | 0.0 | 73 | 87-2846 | 181-1072 |
| 34 | fC-zmro058c07 | NONE | 6714410 | 508 | 1e−142 | 48 | 14-1609 | 198-722 |
| 35 | fC-zmrob73036b05 | NONE | 6714413 | 171 | 8e−42 | 55 | 204-668 | 5-158 |
| 36 | fC-zmro006e07 | NONE | 1652203 | 64.0 | 1e−09 | 36 | 363-605 | 95-175 |
| 37 | fC-zmro033f01 | NONE | 6630702 | 302 | 7e−81 | 54 | 179-1039 | 1-290 |
| 38 | fC-gmst700665347 | SOYMON005 | 6630553 | 257 | 2e−67 | 54 | 311-1162 | 1-292 |
| 39 | fC-zmflmo17255h10 | LIB3205 | 6967639 | 98.3 | 7e−20 | 40 | 126-617 | 188-361 |
| 40 | fC-zmflb73222f01 | LIB3206 | 6682245 | 90.1 | 6e−17 | 41 | 112-408 | 201-296 |
| 41 | fC-zmroB73028f03 | LIB3239 | 2288985 | 72.2 | 1e−11 | 36 | 101-544 | 906-1042 |
| 42 | fC-zmroteosinte034b05 | LIB3204 | 6598859 | 206 | 5e−52 | 48 | 8-772 | 241-501 |
| 43 | fC-zmflb73083d02 | LIB3206 | 6630548 | 81.5 | 1e−14 | 32 | 20-832 | 74-416 |

| Seq | Coding | pep | Complete | |
|---|---|---|---|---|
| Num | Sequence | num | or Partial | NCBI gi Description |
| 1 | 52-240 | 44 | partial | gi\|6907085\|dbj\|BAA90612.1\| |
| | | | | (AP001129) ESTs AU082316 |
| | | | | E3368), 41461(S3973) correspond |
| | | | | to a region of the predicted gene.; |
| | | | | hypothetical protein [Oryza |
| | | | | sativa] |
| 2 | 63-1484 | 45 | complete | gi\|7228459\|dbj\|BAA92419.1\| |
| | | | | (AP001366) EST C74729 |
| | | | | E50675) corresponds to a region |
| | | | | of the predicted gene.; hypothet- |
| | | | | ical protein [Oryza sativa] |
| 3 | 1-1203 | 46 | partial | gi\|4006908\|emb\|CAB16838.1\| |
| | | | | (Z99708) putative protein |
| | | | | [Arabidopsis thaliana] |
| | | | | gi\|7270603\|emb\|CAB80321.1\| |
| | | | | (AL161589) putative protein |
| | | | | [Arabidopsis thaliana] |
| 4 | 129-578 | 47 | complete | gi\|6322653\|ref\|NP_012726.1\|YK |
| | | | | L195W\|Yk1195wp gi\|549742\| |
| | | | | sp\|P36046\|YKT5_yeast hypothet- |
| | | | | ical 47.4 KD protein in PASI- |
| | | | | MST1 intergenic region gi\| |
| | | | | 539215\|pir\|\|S38032 hypothetical |
| | | | | protein YKL195w - yeast |
| | | | | (Saccharomyces cerevisiae) |
| | | | | gi\|486347\|emb\|CAA82039.1\| |
| | | | | (Z28195) ORF YKL195w |
| | | | | [Saccharomyces cerevisiae] |
| 5 | 13-147 | 48 | partial | gi\|6983875\|dbj\|BAA90810.1\| |
| | | | | (AP001168) ESTs AU082174 |
| | | | | (S13676), AU032395(R3986) |
| | | | | correspond to a region of the pre- |
| | | | | dicted gene.; Similar to Arabidopsis |
| | | | | thaliana hypothetical protein |
| | | | | (AF049236) [Oryza sativa] |
| 6 | 2-655 | 49 | partial | gi\|2980788\|emb\|CAA18164.1\| |
| | | | | (AL022197) putative protein |
| | | | | [Arabidopsis thaliana] gi\|7269412 |
| | | | | \|emb\|CAB81372.1\|(AL161563) |
| | | | | putative protein [Arabidopsis |
| | | | | thaliana] |
| 7 | 139-735 | 50 | complete | gi\|3702326\|gb\|AAC62883.1\| |
| | | | | (AC005397) hypothetical protein |
| | | | | [Arabidopsis thaliana] |
| 8 | 140-937 | 51 | complete | gi\|6682251\|gb\|AAF23303.1\|AC01 |
| | | | | 6661_28 (AC016661) unknown |
| | | | | protein [Arabidopsis thaliana] |
| 9 | 2-325 | 52 | complete | gi\|7320718\|emb\|CAB81923.11 |
| | | | | (AL161746) putative protein |
| | | | | [Arabidopsis thaliana] |
| 10 | 1-1437 | 53 | partial | gi\|4455198\|emb\|CAB36521.1\| |
| | | | | (AL035440) putative protein |
| | | | | [Arabidopsis thaliana] gi\| |
| | | | | 7269527\|emb\|CAB79530.1\| |
| | | | | (AL161565) putative protein |
| | | | | [Arabidopsis thaliana] |
| 11 | 248-679 | 54 | complete | gi\|6561955\|emb\|CAB62459.1\| |
| | | | | (AL132964) hypothetical protein |
| | | | | [Arabidopsis thaliana] |
| 12 | 198-1379 | 55 | complete | gi\|6016691\|gb\|AAF01518.1\|AC00 |
| | | | | 9991_14 (AC009991) unknown |
| | | | | protein [Arabidopsis thaliana] |
| 13 | 3-332 | 56 | complete | gi\|7269838\|emb\|CAB79698.1\| |
| | | | | (AL161574) putative protein |
| | | | | [Arabidopsis thaliana] |
| 14 | 2-604 | 57 | complete | gi\|4417289\|gb\|AAD20414.1\| |
| | | | | (AC007019) unknown protein |
| | | | | [Arabidopsis thaliana] |
| 15 | 1-858 | 58 | partial | gi\|6598671\|gb\|AAD25144.2\|AC0 |
| | | | | 07127_10 (AC007127) unknown |
| | | | | protein [Arabidopsis thaliana] |
| 16 | 640-987 | 59 | complete | gi\|4678268\|emb\|CAB41176.1\| |
| | | | | (AL049660) putative protein |
| | | | | [Arabidopsis thaliana] |
| 17 | 1-507 | 60 | partial | gi\|4539400\|emb\|CAB37466.1\| |
| | | | | (AL035526) putative protein |
| | | | | [Arabidopsis thaliana] gi\|7268675 |
| | | | | \|emb\|CAB78883.1\| (AL161549) |
| | | | | putative protein [Arabidopsis |
| | | | | thaliana] |
| 18 | 219-2390 | 61 | complete | gi\|6907089\|dbj\|BAA90616.1\| |
| | | | | (AP001129) hypothetical protein |
| | | | | [Oryza sativa] |
| 19 | 223-1251 | 62 | complete | gi\|6692265\|gb\|AAF24615.1\|AC01 |
| | | | | 0870_8 (AC010870) unknown |
| | | | | protein [Arabidopsis thaliana] |
| 20 | 1-1530 | 63 | partial | gi\|3204103\|emb\|CAA07228.1\| |
| | | | | (AJ006761) hypothetical protein |
| | | | | [Cicer arietinum] |
| 21 | 129-434 | 64 | partial | gi\|6522552\|emb\|CAB61996.1\| |
| | | | | (AL132967) putative protein |
| | | | | [Arabidopsis thaliana] |
| 22 | 1-1764 | 65 | partial | gi\|6041800\|gb\|AAF02120.1\|AC00 |
| | | | | 9755_13 (AC009755) unknown |
| | | | | protein [Arabidopsis thaliana] |
| | | | | gi\|6513917\|gb\|AAF14821.1\|AC01 |
| | | | | 1664_3 (AC011664) unknown |
| | | | | protein [Arabidopsis thaliana] |
| 23 | 1-732 | 66 | partial | gi\|6522547\|emb\|CAB61990.1\| |
| | | | | (AL132955) hypothetical protein |
| | | | | [Arabidopsis thaliana] |
| 24 | 3-1274 | 67 | partial | gi\|6016734\|gb\|AAF01560.1\|AC00 |
| | | | | 9325_30 (AC009325) unknown |
| | | | | protein [Arabidopsis thaliana] |
| | | | | gi\|6091721\|gb\|AAF03433.1\|AC01 |
| | | | | 0797_9 (AC010797) unknown |
| | | | | protein [Arabidopsis thaliana] |
| 25 | 1-1116 | 68 | partial | gi\|6175174\|gb\|AAF04900.1\|AC01 |
| | | | | 1437_15 (AC011437) hypothet- |
| | | | | ical protein [Arabidopsis |
| | | | | thaliana] |
| 26 | 3-539 | 69 | partial | gi\|6143875\|gb\|AAF04422.1\|AC01 |
| | | | | 0927_15 (AC010927) hypothet- |
| | | | | ical protein [Arabidopsis |
| | | | | thaliana] |
| 27 | 3-671 | 70 | partial | gi\|6587865\|gb\|AAF18551.1\|AC01 |
| | | | | 2680_11 (AC012680) unknown |
| | | | | protein [Arabidopsis thaliana] |
| 28 | 241-1152 | 71 | complete | gi\|4049346\|emb\|CAA22571.1\| |
| | | | | (AL034567) putative protein |
| | | | | [Arabidopsis thaliana] gi\|7270148 |
| | | | | \|emb\|CAB79961.1\| (AL161581) |
| | | | | putative protein [Arabidopsis |
| | | | | thaliana] |
| 29 | 1-1011 | 72 | partial | gi\|5042445\|gb\|AAD38282.1\|AC0 |
| | | | | 07789_8 (AC007789) hypothet- |
| | | | | ical protein [Oryza sativa] |
| 30 | 3-590 | 73 | partial | gi\|1001384\|dbj\|BAA10874.1\| |
| | | | | (D64006) hypothetical protein |
| | | | | [Synechocystis sp.] |
| 31 | 1-456 | 74 | complete | gi\|2618699\|gb\|AAB84346.1\| |
| | | | | (AC002510) unknown protein |
| | | | | [Arabidopsis thaliana] |
| 32 | 3-1073 | 75 | partial | gi\|4914426\|emb\|CAB43629.11 |
| | | | | (AL050351) putative protein |
| | | | | [Arabidopsis thaliana] gi\|7270897 |
| | | | | \|emb\|CAB80577.1\|(AL161594) |
| | | | | putative protein [Arabidopsis |
| | | | | thaliana] |
| 33 | 117-2939 | 76 | complete | gi\|6539560\|dbj\|BAA88177.1\| |
| | | | | (AP000836) hypothetical protein |
| | | | | [Oryza sativa] |
| 34 | 2-1672 | 77 | complete | gi\|6714410\|gb\|AAF26098.1\|AC01 |
| | | | | 2328_1 (AC012328) unknown |
| | | | | protein [Arabidopsis thaliana] |
| 35 | 183-686 | 78 | complete | gi\|6714413\|gb\|AAF26101.1\|AC01 |
| | | | | 2328_4 (AC012328) unknown |
| | | | | protein [Arabidopsis thaliana] |
| 36 | 363-605 | 79 | partial | gi\|1652203\|dbj\|BAA17127.1\| |
| | | | | (D90903) hypothetical protein |
| | | | | [Synechocystis sp.] |
| 37 | 179-1054 | 80 | complete | gi\|6630702\|dbj\|BAA88548.1\| |
| | | | | (AP000969) hypothetical protein |
| | | | | [Oryza sativa] |
| | | | | gi\|6721539\|dbj\|BAA89569.1\| |
| | | | | (AP001073) hypothetical protein |
| | | | | [Oryza sativa] |
| 38 | 281-1165 | 81 | complete | gi\|6630553\|gb\|AAF19572.1\|AC01 |
| | | | | 1708_15 (AC011708) unknown |
| | | | | protein [Arabidopsis thaliana] |
| 39 | 3-716 | 82 | partial | gi\|6967639\|emb\|CAB72629.1\| |
| | | | | (AL139074) hypothetical protein |
| | | | | Cj0145 [Campylobacter jejuni] |
| 40 | 1-969 | 83 | complete | gi\|6682245\|gb\|AAF23297.1\|AC01 |
| | | | | 6661_22 (AC016661) hypothet- |
| | | | | ical protein [Arabidopsis |
| | | | | thaliana] |
| 41 | 2-565 | 84 | partial | gi\|2288985\|gb\|AAB64314.1\| |
| | | | | (AC002335) hypothetical protein |
| | | | | [Arabidopsis thaliana] |
| 42 | 2-778 | 85 | partial | gi\|6598859\|gb\|AAF18713.1\|AC01 |
| | | | | 0556_9 (AC010556) hypothetical |
| | | | | protein [Arabidopsis thaliana] |
| 43 | 2-868 | 86 | partial | gi\|6630548\|gb\|AAF19567.1\|AC01 |
| | | | | 1708_10 (AC011708) hypothet- |
| | | | | ical protein [Arabidopsis |
| | | | | thaliana] |

The entries in the Seq Num column refer to the corresponding sequence in the sequence listing.

### Seq ID

The Seq ID is the name of the insert sequence in a particular clone found in the SEQdb databases (Monsanto Company, St. Louis Mo.). Each Seq ID entry in the table refers to the clone whose sequence is used for the sequence comparison whose scores are presented.

### Library

The entries in the “Library” column refer to the cDNA library from which the clone is obtained. The libraries are as follows: The LIB3205 cDNA library is from Zea mays L. (Mo17, USDA Maize Genetic Stock Center, Urbana, Ill. U.S.A.), unpollinated ear with silk. The LIB3206 cDNA library is from Zea mays L. (B73, Illinois Foundation Seeds, Champaign, Illinois U.S.A) from unpollinated ear and silk. The SOYMON007 cDNA library is generated from soybean cultivar Asgrow 3244 (Asgrow Seed Company, Des Moines, Iowa U.S.A.) seed tissue. The LIB3239 cDNA library is from Zea mays L. (B73, Illinois Foundation Seeds, Champaign, Illinois U.S.A) from root at the V2/V3 stage. The SOYMON014 cDNA library is generated from soybean cultivar Asgrow 3244 (Asgrow Seed Company, Des Moines, Iowa U.S.A.) seeds and pods, which are harvested from plants grown in a field in Jerseyville 15 days after flowering. The SOYMON015 cDNA is generated from soybean cultivar Asgrow 3244 (Asgrow Seed Company, Des Moines, Iowa U.S.A.) seed tissue harvested 45 and 55 days post-flowering. Seedpods from field grown plants are harvested 45 and 55 days after flowering. The SOYMON024 cDNA library is generated from soybean cultivar Asgrow 3244 (Asgrow Seed Company, Des Moines, Iowa U.S.A.) internode-2 tissue harvested 18 days post-imbibition. The LIB3204 cDNA library is from Zea mays L. ssp. mexicana from root tissue. The SOYMON012 cDNA library is generated from soybean cultivar Asgrow 3244 (Asgrow Seed Company, Des Moines, Iowa U.S.A.) leaf tissue. The SOYMON005 cDNA library is generated from soybean cultivar Asgrow 3244 (Asgrow Seed Company, Des Moines, Iowa U.S.A.) hypocotyl axis tissue from seeds 6 hour post-imbibition. In some cases, no library information is given and “NONE” is listed.

### NCBI gi Number

Each sequence in the GenBank public database is arbitrarily assigned a unique NCBI gi (National Center for Biotechnology Information GenBank Identifier) number. In this table, the NCBI gi number which is associated (in the same row) with a given clone refers to the particular GenBank sequence which is used in the sequence comparison.

### Blast Bit Score

Bit score for BLAST match score that is generated by sequence comparison of the full length with the GenBank sequence listed in the Description column.

### E-Value

The entries in the E-Value column refer to the probability that such matches occur by chance.

### % Ident

The entries in the “% Ident” column of the table refer to the percentage of identically matched nucleotides (or residues) that exist along the length of that portion of the sequences which is aligned by the BLAST comparison to generate the statistical scores presented.

### Qstart-Qend

The entries in the “QStart” column refer to the location of the nucleotide in the designated clone that first matches with the designated NCBI sequence QEnd” column refer to the location of the nucleotide in the designated clone that ends the match with the designated NCBI sequence.

### SStart

The entries in the “SStart” column refer to the location of the amino acid in the designated NCBI sequence that is first matched with a sequence in the designated clone. SEnd” refers to the location of the amino acid in the designated NCBI sequence.

### Coding Seq

The entries in this column refer to the nucleotide where translation begins and ends

### Pep Num

The entries in this column refer to the number of the translated nucleotide sequence in the sequence listing

### Complete or Partial

The entries in this column describe the relative placement of the longest ORF and the BLAST results. A sequence is listed as “partial” if the query sequence contains a complete open reading frame 1) with the starting codon (ATG) located greater than 30 by from the 5′ end and the subject sequence does not contain an ATG 2) the query sequence contains no ATG or start codon or 3) the query sequence ATG position is greater than 30 by from the 5′ end and there is no matching subject sequence. A sequence is referred to as “complete” if the query sequence contains a complete open reading frame and 1) the query sequence ATG position is less than 30 bases from the 5′ end and there is no matching subject sequence 2) the query sequence ATG is greater than 30 by from the 5′ end and the subject sequence does not have an ATG

### NCBI gi Description

The “NCBI gi Description” column provides a description of the NCBIgi referenced in the “NCBIgi” column.

### Example 2

SEQ ID NO: 1 through SEQ ID NO: 43 correspond to the sequence of the entire cDNA inserts of the clones set forth in Table 1. The deduced amino acid sequence for these DNA sequences (SEQ ID NO: 44 through SEQ ID NO: 86) is determined using the Translation program in LifeTools™ (Incyte Pharmaceuticals Inc., Palo Alto, Calif.), Finishing Manager (Millennium Pharmaceuticals, Cambridge, Mass.) or similar translation program.

### REFERENCES

All references cited above are incorporated by reference in their entirety. In addition, these references can be relied upon to make and use aspects of the invention.

1-26. (canceled)

27. A transformed plant comprising a nucleic acid molecule which comprises:
(a) an exogenous promoter region which functions in a plant cell to cause the production of an mRNA molecule; which is linked to;
(b) a structural nucleic acid molecule, wherein said structural nucleic acid molecule comprises a nucleic acid sequence, wherein said nucleic acid sequence shares between 100% and 90% sequence identity with a nucleic acid sequence selected from the group consisting of SEQ ID NO: 1 through SEQ ID NO: 43 and complements thereof, and
which is linked to
(c) a 3′ non-translated sequence that functions in said plant cell to cause the termination of transcription and the addition of polyadenylated ribonucleotides to said 3′ end of said mRNA molecule.

28. The transformed plant according to claim 27, wherein said nucleic acid sequence is the complement of a nucleic acid sequence, wherein said nucleic acid sequence shares between 100% and 90% sequence identity with a nucleic acid sequence selected from the group consisting of SEQ ID NO: 1 through SEQ ID NO: 43.

29. The transformed plant according to claim 27, wherein said nucleic acid sequence is in the antisense orientation of a nucleic acid sequence that shares between 100% and 90% sequence identity with a nucleic acid sequence selected from the group consisting of SEQ ID NO: 1 through SEQ ID NO: 43.

30. The transformed plant according to claim 27, wherein said nucleic acid sequence shares between 100% and 95% sequence identity with a nucleic acid sequence selected from the group consisting of SEQ ID NO: 1 through SEQ ID NO: 43 and complements thereof.

31. The transformed plant according to claim 30, wherein said nucleic acid sequence shares between 100% and 98% sequence identity with a nucleic acid sequence selected from the group consisting of SEQ ID NO: 1 through SEQ ID NO: 43 and complements thereof.

32. The transformed plant according to claim 31, wherein said nucleic acid sequence shares between 100% and 99% sequence identity with a nucleic acid sequence selected from the group consisting of SEQ ID NO: 1 through SEQ ID NO: 43 and complements thereof.

33. The transformed plant according to claim 32, wherein said nucleic acid sequence shares 100% sequence identity with a nucleic acid sequence selected from the group consisting of SEQ ID NO: 1 through SEQ ID NO: 43 and complements thereof.

34. A transformed seed comprising a transformed plant cell comprising a nucleic acid molecule which comprises:
(a) an exogenous promoter region which functions in said plant cell to cause the production of an mRNA molecule; which is linked to;
(b) a structural nucleic acid molecule, wherein said structural nucleic acid molecule comprises a nucleic acid sequence, wherein said nucleic acid sequence shares between 100% and 90% sequence identity with a nucleic acid sequence selected from the group consisting of SEQ ID NO: 1 through SEQ ID NO: 43 and complements thereof,
which is linked to
(c) a 3′ non-translated sequence that functions in said plant cell to cause the termination of transcription and the addition of polyadenylated ribonucleotides to said 3′ end of said mRNA molecule.

35. The transformed seed according to claim 34, wherein said nucleic acid sequence is the complement of a nucleic acid sequence that shares between 100% and 90% sequence identity with a nucleic acid sequence selected from the group consisting of SEQ ID NO: 1 through SEQ ID NO: 43.

36. The transformed seed according to claim 35, wherein said exogenous promoter region functions in a seed cell.

37. The transformed seed according to claim 35, wherein said nucleic acid sequence shares between 100% and 95% sequence identity with a nucleic acid sequence selected from the group consisting of SEQ ID NO: 1 through SEQ ID NO: 43 and complements thereof.

38. The transformed seed according to claim 37, wherein said nucleic acid sequence shares between 100% and 98% sequence identity with a nucleic acid sequence selected from the group consisting of SEQ ID NO: 1 through SEQ ID NO: 43 and complements thereof.

39. The transformed seed according to claim 38, wherein said nucleic acid sequence shares between 100% and 99% sequence identity with a nucleic acid sequence selected from the group consisting of SEQ ID NO: 1 through SEQ ID NO: 43 and complements thereof.

40. The transformed seed according to claim 39, wherein said nucleic acid sequence shares 100% sequence identity with a nucleic acid sequence selected from the group consisting of SEQ ID NO: 1 through SEQ ID NO: 43 and complements thereof.

41. A method of growing a transgenic plant comprising
(a) planting a transformed seed comprising a nucleic acid sequence, wherein said nucleic acid sequence shares between 100% and 90% sequence identity with a nucleic acid sequence selected from the group consisting of SEQ ID NO: 1 through SEQ ID NO: 43 and complements thereof, and
(b) growing a plant from said seed.

42. A substantially purified nucleic acid molecule comprising a nucleic acid sequence, wherein said nucleic acid sequence shares between 100% and 90% sequence identity with a nucleic acid sequence selected from the group consisting of SEQ ID NO: 1 through SEQ ID NO: 43 and complements thereof.

43. The substantially purified nucleic acid molecule of claim 42, wherein said nucleic acid molecule encodes a maize protein or fragment thereof.

44. The substantially purified nucleic acid molecule of claim 43, wherein said nucleic acid molecule encodes a soybean protein or fragment thereof.

45. A substantially purified nucleic acid molecule that encodes a protein comprising an amino acid sequence that is at least 70% identical to an amino acid sequence selected from the group consisting of SEQ ID NO: 44 through SEQ ID NO: 86.

46. The substantially purified nucleic acid molecule according to claim 45, wherein said nucleic acid sequence encodes a protein comprising an amino acid sequence that is at least 90% identical to an amino acid sequence selected from the group consisting of SEQ ID NO: 44 through SEQ ID NO: 86.


SEQUENCE LISTING (86 sequences)

SEQ ID NO: 1 - DNA, 463 bp, Zea mays
  CDS (52)..(240)
ccacgcgtccgcaacaatttgatcaggcaataacctgacgaccctggtttgttctgtgaatgcattttcgtcgtgcttcaacaacagggaccggactacatggtccgcaacgcaaggaggtccatgctggaggagctggaggggatgctggagatcgtggagcctcagccgccggggaagccgaggacgcttagccgcaggaggttcgatctcccagaaggcgtagccatcgaaaaggagacgcgggaggcgggcaagtgaagtgcatcggtgggtggtggcatgcttgggcagtttggtatatgcgcgatgcggactatagagtttgagtttgtgctgctaccgaaccgtggaccaccttaccccgttgagatgtttgatagctggactttttgtgctgttgtatttgtagaccagaacgttgttctttgtaataaattggcgtgtgctgctttttgctaat

SEQ ID NO: 2 - DNA, 1702 bp, Zea mays
  CDS (63)..(1484)
attcggctcgagtttgatccgagcccacagtctctcctcgggcccaccgcgtccgaccggcgatggcaaagaccccgtcgttcgcggtggcggcggtcgccggaggccgcgggccggttcacaaccggacccagctcctcctcctcctcctcgtggccgtcgcagcctccgcatccacagcagggttcctcctccgcggtgccctgcgagacccttgcgacggccgtggggaccccgccgccctcaacaccgccgtcgccagcgggagtcccctcgggttcatgaggtccaagctcgtgctcctcgtctcccatgagctctccctctctggtggtccacttttactgatggaattagcatttcttctgaggcatgttggctcgcaagtggtgtggataacaaaccagagatcacaagaaacaaatgatgtcacatatagcttggagcataggatgttgaaccatggagtgcaggttttaccagctagaggacaggaggcagttgatattgctctaaaagctgatctggttatcttaaacactgctgttgctggcaagtggcttgaccctgttctgaaagatcatgttcctaaagtccttccgaagattttgtggtggatccatgaaatgcgtgggcattactttaaggttgaatatgtcaaacatcttccctttgttgctggagccatgattgattctcatacaacggctgagtattggaatagcaggactagcgatcgcctgaaaatacagatgccacaaacttatgttgttcacctggggaatagtaaagaactaatggaagttgctgaagacaatgtcgcaagaagagtcctacgggaacatattcgtgaatcccttggagtacggagtgaggatctcctgtttgcaataataaacagtgtatcacgaggaaagggacaagacttatttcttcaagcattttatcaggctttgcagctcatccaacacgagaaacttaaagtgcctagaatacatgctgtagttgtgggaagtgatgttaatgctcagaccaaatttgagactcagttacgtgactttgtggtgaagaacacgattcatgaccgtgtccattttgtgaacaagacattggcagtggccccttacttggcagcaattgatgtgcttgttcagaattctcagggccgtggagaatgctttggaaggataacaattgaagcaatggcattcaagttgccagtattgggcacggctgctggagggaccacggagatcgtcctggacggctcgactggccttctgcatcctgctgggaaggagggcgtggcgcctcttgcaaagaacatcgtcagactcgcaagccacgccgagcagagggtctccatgggggaaaagggctatggcagggtgaaggaaatgttcatggagcaccacatggctgagaggatcgcggcggtgttgaaggatgtcctgaggaaatcacaggagcactccaggtcttgagctttgccgtgcccatcagcttgcgctaacatgttgaactagattttacgggctacgcctacgtggttcaggctgtaaactgtagattgcactctgttggtctactttttcacattcatgttttacctattaggccatgtccgattctattccaattcatataggttctatttcaatccatatagattaagagggattgaggagatttcaatcttagt

SEQ ID NO: 3 - DNA, 1430 bp, Zea mays
  CDS (1)..(1203)
ccacgcgtccgtctaattatagaaaagaatagagactacacagttgattatagcagctcatcttttggactatcaggtgctagttatatatcatcccccatgagggaaacagagcagtcaaagactagttttgaccagttttattctaatgccaatttccagttgtatttgtccttctgcaactttgacaaggcaatgttcttgggtttctttcatgagctatccgagcttccctttgaactgcaaagaaaagctgtcagagatttgaagacttctctgagcggtgaaaatgaaatttggcattctatggtctacaatgggttttttgaagcattccatgaattcctcaagaatgacagtggaattcacacactgcaagctcgaagggctgggattcagttttttcttgctttcctttctagtggcagggctcgaattccttcagtttgtgaagatgtggtacttctgattgcatcactacatgattccgagttcaaacaggaggctcttctgattgtacatgaactgcttcaggaaccaagctgtccaaaatctagtctcatggcctccattctttctccttcagtgtttggagctttggacagtggagaaaccaagtgcctggacctcgctctgcagatcatctgcaagatttcatctgataatgatataaaatcttaccttctttcgtccggaatagtgtcgaggttatctccgctccttggtgaaggaaagatgacagaatgctctttgaagattctacggaacttgagtgacgtgaaagagaccgcagggtttataatcagaacaggtaattgcgtcagctccatttcagatcatctggacactggaagccacagcgaacgtgaacatgcggtggtcatccttctaggggtatgctcccacagtcctgaggtttgttcactttccatgaaggaaggcgtcatcccagcccttgtagacttatcagtgagtggaaccaaggtggcaagggattgctcggtcaagttgcttcagcttctgaggaacttcaggcgatgtgaccagttcagcagttcatgctcaagagagcttgctgtcgatcatgtttcagagaacactcgcaatggttcaatttgcatgcagccgatatcaaagtcagcccggtatatttcaagaaagctcaaccttttctcaaaacctcggtcgctgaccctggcttgagaaatggaaggggtcggttggatcgagccctattccgcagcgctaactgccagatgtacagatagtagcaggtagcgttcgtcgagatgaaatgtttgtgggaggctttttaaaactcaccatgtatttcaagagtttttattagttttttttggattttctttactggcgctacaaacagtagatgtatgactgttcgagctggaaacctgtgcgctttttatcgt

SEQ ID NO: 4 - DNA, 1048 bp, Zea mays
  CDS (129)..(578)
ccacgcgtccgctccagcgttgagccaactccgccgccctgtcgttttcgcctctccctagcatttaccggaagaaacgagcgtgaccgccgccttctcccgctcccgctcccgctctgcatctctccatgggccagatcgagagccaggtcactcctccagcggaggagccttctccacccaccgtggagccgtcgccgtcgtctcctgccccgcctccgtcttcccttgaggcgattgctgcagaagccatgtcatttgatgaggatgacactgaggagtcaatcgatgtgaaggtacagaaagctctggactgtccatgtgttgctgatttgaaaaatggtccttgtggaggtcaattcgtcgatgcattttcctgctttctcaggagcagagaagaagagaagggatcagattgcgtgaaacccttcatcacattgcaggactgcatcaaagcaaatccagaggcattctctaaggagattctggaggaagaggaaaatgatgaggaggcagataagtccaacctgaaagttagagctccatcatggtccagagaatccaaacctaaggtttgagattgcttggtattaactgatagtgatattcatccagtgtgcagactcaccttattcaactataagggcttgttcgctttcctctcaatccatgtgatctcaatccatgtggattgggtgggattggatgagtttaaatcctgaacaagtcaaaatccttcatattttttctaatctcatccaatccacatgggacaggaataaccgaacaagccctaagtttgccagtagtagactgatttcagagagcgaggtatcataaattgattaacagaaatacccatattttgtgtataagaaatgcttcatggtatccacaattttagaatgtacatgtcatttcaagtatcaatcgagaactgatgaacttgtaattcgattcctagatacagggcatatttgatcaccactgtattattatactatgaaatatacatatgattatcataatctaggttatgaactataat

SEQ ID NO: 5 - DNA, 816 bp, Zea mays
  CDS (13)..(147)
ccacgcgtccgggggcagacgcggtgggagatgttcgacctgctgtcgtcgctgccgtccacgtcctcggcctcgtcaacgacgaccgtgagctccacggcgtcctccggcgcgccgccgccgagccgcccggactggatgctcttctgacgagggagggacggcggccccgcaccggccggccaccggcgcgaaagcgaaaggcgagtgcttgatgcggtgcgatgcttgtgtgccaaaaattgcaccccaaatctggtagctcgtcgagtgtcctgatgaggccatgattgggcaggcgggggggctatctagctttggccacacagctgcattgcgttggccgttgggctgcatggtaccgtaccagccaattcgagttcatcaatcatgtggtgtgagagtatttttcttcttcacctctcctttttctttctttccttttgagcttttttttccctcctcctcctcctcctcctcttcttcttgttcatggaaggtttgggattcttttggagtgcatactatactattcagatagagaaacagaaagatggagttggttagggcaagtgaaacgtcttcagatagggaaataaataaaactagtcggtgtccaaactctgaagaaacgaacaaagaggtgcatggattgttagcttatcacactgttgtttgagagtgatgagagatgtatcgatcaagaggccacttggtcggtagtctgtcactccttgttgctgccgagtttcttcctcctcctcatcatcctgtataatgtaatcaagcaataatacaaaataaaaaagcgttttg

SEQ ID NO: 6 - DNA, 1040 bp, Zea mays
  CDS (2)..(655)
ccacgcgtccgcaattgggcgcctgatatctccaagctcgggctgttctggcacatcctccccattccctgaccctgagatgcaggcttcttcacgcagcgctttacgcttgttcccagttcgtgagccccctaagatattggatggcgagggcgttgcgacacagaagttgatacctcgccatatgcgcaacggtgggtccctcttggatggccagatctcagcagctgtaccagttgtggacttctctgcccgacttcaacccaacgagcacgcaatggatcaccgggtgtcattcgagttgaccgtcgaagatgtcgcgcgctgccttgagaagaagactgcaatctccggggattctggcacggcatcattccaccttgcaccgaccggcagcggcgaccaccacagagaatccaacgaggcaagggcagggctctacgtcgacgaatcataccatgacttgcccgagaaagcgaggcggtccctgtccctgcgcctggccaaagagttcaatttcaacaacgtcgacgtcggtagcgtggagccgagcgtgggatccgactggtgggcgaacgagaaagtcgccgggatgacaactgagccaaaaaagaactggtctttccacccggtggtgcagcctggggtcagctaacctttgcactgaccataatgatcttatacagggatgaaggaattagcttcttctgcttctgcttctgcttcagtggtcgattccttagggatgacttgccagcttgttctaaacatgcagctagtggtaagttctggatttagatagtaataagtaatggggtgtccaccgtctaatatcgtcatgggatactaaaggtttttattctgggagatgtgacaaaccgcaaactctttggttaggggtcggtcggaactgtcctgttcgtctgttttatcctgataccggtgctgtgttgtgtatgtacataatgaagtagttttttttaaaaaaagtcaattaaaagggtagaacagtttccttcgataagttgccagatgctacc

SEQ ID NO: 7 - DNA, 1145 bp, Zea mays
  CDS (139)..(735)
attcggctcgagatttttccctagtaattttccccttgtgcggctattccaccccacgacgaacctgcgcatctctctctctctcgcgcgcgccgcttttctctcagccagccctgcagctctttttctccgcccacaatgggtaaagctaaaaagggaccaaagtttgctgttatgaagaagattgtcacttccaaagcaatcaaaagctacaaagaagaggttttgaacccagaaaagaagaatcttatgaaggaaaagttacccagaaatgttccaactcattcttcagcgcttttctttcaatacaacactgcactgggacccccttatcgtgttttggtggacaccaacttcatcaatttctcgatccaaaataaattggatctggagaaagggatgatggactgcttatatgcaaaatgcaccccttgtattacggactgtgtgatggcagaactcgagaagttaggccaaaaatatcgtgtagctctaaggattgccaaggatcctcgatttgagagaatactatgtactcataaagggacctatgctgatgactgccttgttgagagagttactcagcacaagtgctacattgttgcaacatgtgatcgggacttgaagaggagaattcggaagattcccggtgttccaataatgtacatcaccaaacgcaagtactcgattgagcgattgcctgaagcaacaattggtggagctccaagaatttgatgcagtaaaactaaaacaggcagagctggctataacaataatattgatactaatttcttggtcgtgaagtatgaaatcctgctacagtacctagcatattcggtcttttgctggctgggctgaattctatcggaagcttgagacttgaacaaaagcagcacttcaatggtttatacctgaaattttcttagtgtaagttggtagatttaggttctgtcaaattttgttttcctcatattgcctatctaatgtatcatggtaaaccttggagagtgtctgaactatgatgacattttcatagttctgggcttgtggcaattaactctcttgaaacgatactttttttgttgcaagagaaaaaccttattttccttgaatctaaattttattagtttcaagcttgacatgcc

SEQ ID NO: 8 - DNA, 1182 bp, Glycine max
  CDS (140)..(937)
attcggctcgaggtaacagtctgtgtctgtgtgtgtgttcagtttaacaaatggatcaagctgtccactgatctgtaaccgtggtggactcgacaagacaacaacaatattcagaacattaacaagaccacaccacaccatgagcgtcatcgacattctcaccagagttgattccatttgcaaaaagtacgacaaatacgacgtccaaagccaaagggactccaatctctcctccgacgatgcattcgccaaactctacgcctccgtcgacgccgacattgaggccttacttcagaaagcagataccgcttccaaggagaaaagtaaggcatccactgtggcgatcaatgccgagattcgtcgaaccaaggctaggttgttggaggaggttcccaagttgcagaaattggctatgaaaaaggtaaaggggctttcatcacaagaatttgctgcccgtaatgatttggctcttgcattgccggataggattcaagctatcccagatgggacccctgcagcatccaaacaaactggaagttgggcagcttcagcctcacgtcctggaattaaatttgatacagatgggaaattcgatgatgaatacttccaacaaactgaagaatcaagtggattcaggaaagagtacgaaatgcgtaaaatgaaacaggatcaaggtttggatatgatcgcagaaggattggatactttgaaaaacatggcacatgatatgaatgaggaactggatagacaagttccactgatggacgagattgatactaaggtggacagggcatcttctgaccttaaaaataccaatgttagacttagagatacagtgaaccagcttcgatccagtcgaaacttttgtattgatattgttttgttgattataattttgggaattgctgcttatttgtacaacgtgctaaagaaatgatacggcatagaagaccattagatcaatttgtatggcttatcagtatttcttgtattttcttggtaatatcgtggtgtgactcgtgtctgcttctttttaaaacaatatttttggacttgttcctgcaacttgagagatgcacttgtcctttatcaacagatctatatgtcgccgtattatgtcaaacagtgtagatttgtatttatacttgtgtaatatactaattcaatgtttgccttgaggat

SEQ ID NO: 9 - DNA, 654 bp, Zea mays
  CDS (2)..(325)
ccacgcgtccggattcctgaagtgtgctatcttgacaaaacctcagtcatatggtgtgttgctgcagttaccagctcctcagcttgagaatgccttgagtaagaacccgacactgaagacgcccttggctgagcatgccgagcagccaaatattcggtcgacacttccaaggtctaccttggtggttctgggtcttgctgaagatcaaccacagcaaccagcagtaacacaggtgcagagcagccaaaaccaggctgcggaaaccagtagctctgctgctgatacggctacagaagtaactcaggaatcttctggtgctagctaacatcttttatgccgaaatgggctaatgcttgcaggttgtatgccatctatcattttcatctctggagtgatgttggtgattgatgaaatgggctaacgcttgcaggttgtgtaccatctgtctctgagggtctaagatgtgtttttctgtgtgatgcgtctcattggacgggtgttagctgctgcatctggacgcctaaatgagaactaagttatttagttgctgggggtaattctaattttcggtggtgcacagggaccattgtaaagtgaagaggttaacattatatatcatccttaaataggaaaagggcaaacaagtgtttgttg

SEQ ID NO: 10 - DNA, 1599 bp, Glycine max
  CDS (1)..(1437)
attcggctcgagtttgaatctctggaaattgagatggcgaacgagaacgaacccgcgaagctgctgttgccataccttcaacgcgccgatgaattgcaaaagcatgaaccactcgtcgcttattactgtcgattatatgcaatggaacgggggttgaagattccgcaaagtgagcgcacgaagaccactaatgctcttctggtttcgctcatgaagcagcttgaaaaggataaaaagtcaatccagttggggcctgaagacaatttatatctcgagggatttgctttgaatgtgtttggaaaagcagacaagcaagatcgtgctggaagagcagatttgactacagcaaaaacattttatgctgccagtatcttttttgagattcttaatcaatttggagcagttcagcctgatctggagcagaaacaaaaatatgctgtgtggaaagcagctgaaataagaaaagctttgaaagaaggaaggaagcccacagctggcccccctgatggtgatgaggatctgtcagttcctttgagttcttcaagtgatagatatgaccttgggactactgaaaatactgtttccagtcctggaccagaatctgattcatcaagaagttatcataaccctgctaactaccagaatctgccaagcattcatcctgctgctcctaaatttcatgatactgttaacgaccaacattctgctaatattccatcatctatgccatttcatgatagagtagataataacaagcattcttccgttgtttctccatcatctcactctttcacacctggagtttatccttcccaagactaccattctcctccaccttcccgagactatcattctcctccaccatcccaagactatcattctcctccatcgtcccaagattatcatcctcctccaccatcccaagattaccatcctccaccgtcccaagattaccatcctccacctgctagatctgaaggttcttattctgagctctacaatcatcagcaatactcaccagagaattcacagcatttaggtcctaactacccttctcatgaaacttcctcttattcttatccccattttcagtcttatccaagttttacagaaagcagccttccatcagtcccatcaaactatactcattatcaaggatcagatgtttcatattcttcccagtcggctccgctaactacaaaccattcatcaagtgctcaacacagtagcagaaatgaaactgtggaacctaagccaacaactactcaggcataccagtacgacagtaactaccagccagcacctgaaaaaatagcagaggcacacaaggctgcaagatttgccgttggggcactggcatttgatgatgtctcagttgcagtagacttcttgaagaaatcacttgagttgctgacaaatccatcagctggccagtaaaattttttgtaaatcgaaatgtggatttcggtggtgttttctaccttttatttgcatgtcttgttttgtatattaggtacatcatgcatttaaagtgtgaaaaaatttgggaagatgaacggaaattataaaatctgctgggatctgcaatctttcttgttt

SEQ ID NO: 11 - DNA, 1807 bp, Zea mays
  CDS (248)..(679)
ccacgcgtccgtataaatcagcccccttcctccacggtcgcgtttcacgggcgtagagagagagagagagagagagagcgcgagcgagcgaagagggcgtgagagaggagggaggccaagccgtacgcgtcgtctcctgctgtttctccccatccctttcctgcctcggcttgcaccttcaaccttacttaaagctctgacctcaaactgaaagggacctgcgaagagttttttttgcggcagtacaatggaggtatttggcaaatctgtgattgctgagcccagcaatgtgattttcttgtccgcgatccttaacacagaagggtcaaaccctagtcacaagtgtgacaagaggtgccagagcgagcgcattttggggaacatgtaccgttgcaaactgactgaaaccactcacatctgtgacaaaaactgtaaccagaggattctatatgacaaccataactcgctctgccgagtgagtgggcagctttttccgctctctccactggagcagcaagcagtgaggggcatccgcaggaagcatgaagtggacagcagtgaaggttgctgctttaagcgcaggcgcggcgcacagctgcatccttcccccttcgagaggtcctactctgctgtgtatccaatcccgagccaggttggagatggcatggacatgagctagagctaatgagacttccttttcttcggccagccacctaatttcgttctgtctaccctttttccctacgattacaagatggaactataagagcttccttctttttagacttatatgagactccttagatgcaccgaataaatgttcatgtagtatctattctgtttcatgggaccttgttgtaactcgtgaactttgattgaacgcctattttgatttagtggtactttcagataatcattagattcttctcttttttttgtcacacttccaatttagttattttatgtgctttactttttgttcaactaatttgccttctaattttaatcctttgatgcaatttaatataattgagaggtttacatttgtccgccaatgtaagtaagtgaaggattctttggtcctgtaatccaggcacagggaactgacatgcctttgctgactgaagtttttgtacagctgttactagtgttttgcttgacaagttcctaatcaagcttgaatcatttacggtttgttcccatatagctcaattctatctgttgcttaactcttttttttacaggttatgtttctaacgcttaatgttttcctatgttcttttttgtcttaacatttgttgtatgcaggaccatccaggttctaatcgttttttgggaaatgttttgttcttttttatataactatattgctaacttaatttaatttgttgattaatttgcctttctttgtttttgcaggtgaacatctctgattccattttcatctgttgggtggcaagaaactaatcaaggagtctctctcttctcattctctgcgtggaggactgtttagctgtagctgagtggcatgctttccagaacataaattggtggaagagaagtttgatgctaccactttgcgagaaggaccgaaccaacacaataacatcaaacttagatactgcagtttaccaactttttcaatattgaagattgaagagcagcaagatcagcctcttccatatgcagtttgaagaaatcctttttaaggtctattagactagaatccttccggcaatcttgccttcaccccaagcatgctgcataccttgtaatgagtgtggtacttgacgactgttaccaaagt

SEQ ID NO: 12 - DNA, 1524 bp, Zea mays
  CDS (198)..(1379)
ccacgcgtccgggcaggtcagtcagtagtggatagctccgctcgcggtcgcgggcggctcgagtcgcaccccagatcccaatgcgccccgccgcgccgcactaggccgcggaacggaagctaggtgcgtcacacgccggggccggtcgcggtcaccggaccggacgcacaggggcgccgtacgtgcagtcatcggccatgaaggtcctcgtcctcgccgtgctggcgctcgtcgccgccgcctccgccgctggtcagggcgaggaggggggcgggccgccgctgccgttcgcgttgggcgcggcgccggcgggctgcgacgtcgcgcagggcgagtgggtgcgcgacgacgacgcccgcccatggtaccaggaatgggagtgcccctacatccagccgcagctgacgtgccaggcgcacggccgccccgacaaggcgtaccagagctggcgctggcagccgcggggctgctcgctgcccagcttcaacgcgacgctgatgctggagatgctgcgggggaagcggatgctgttcgtgggtgactcgctgaaccgggggcagtacgtgtccctcctctgcctcctgcaccgggccatccccgacggcgccaagtcgttcgagacggtggactcgctgagcgtcttccgggcgaagaactacgacgccaccatcgagttctactgggcgccgatgctggccgagtccaactccgacgacgccgtggtgcactccgccgacgaccgcctcatccgcggcgcgcccatggacaggcactacagcttctggaagggcgccgacgtcctcgtcttcaactcctacctctggtgggtcgccggggacaaaatccagatcctgaggggcgccgacaacgacccgagcaaggacatcgtggagatgaagtcggaggaggcctaccggctggtgctgcaccaggtggtccggtggctggagcgcaacgtggaccccggcaagtcacgggtgttcttcgtcaccgcttcgccgacgcacacggacggcagagcgtggggcgacgacgacgcggagggcagcagcaactgctacaaccagacgtcgccgatcagcgccgcctcgtcctaccgtggcggcacgagccgggagatgctgcgcgcaacggaggaggtgctggccacgtcgcgggtgcccgtcgggctggtcaacatcacgcggctgtccgagtaccgccgggacgcgcacacgcagacctacaagaagcagtgggtggagccgacggccgagcagcgcgccgaccccaggagctacgccgactgcacgcactggtgcctccccggcgtgccggacacgtggaacgagctgctctactggaagctcttcttccccagcaacgatcaggtcctctgatggtgaattggtgcctgcatacatacgcctggaaaccaactgctaggggctagcccaaagaaaatgtgtctagatgaacaaagacatggggttcaaatgttttgttttgatttcggatatggacgctgtcgatgctcttgttctc

SEQ ID NO: 13 - DNA, 847 bp, Zea mays
  CDS (3)..(332)
ccacgcgtccgtgcaattctatgaatatgcagttatcgcaattgcctttagattgtaaaaggctgacttatgatgctcttgaaggagctaacgtcactccgacgtccttttacaacattggtgatcttgagattcaagataatctagcacgagtatgggtagacattggtattcatgagccattgcttctggacatcctgcttaatgccttaacaacaataagttcagatcatgttggtattaagcaagtacagtttggagggtcagagtttttgaactggagcgaggacttgaagacagaagaagttggatatagtgtgtgcaaaatctaaatagcacaatcccgagatttgcagaagcagctacctttttagctaaacgcaaatgcaacatctggttcgaagctagggttttaaccaccttgatcctgcaaacgagaagaagaaaatggtgacaacacgatgaaaaattgacgtccaaacatattaacttctgcttgaaaagaggttgccgtccttacaggactctgctagctacacatcaacacatcattggtaatccttctgcatacatgtaataatataatataactagattatctgatgtccaacgtgtattgactctacttagggctgaacataatacttatggctcgttagttcgcttggctcgacttagtttatttcaattttgttacgagctaagttagtattttagctcggtttgttaacgagccatctcgttagctcaaacgagctatcatttatcaacaaaataaagtcttcacttatgttggatgaactaataagtgattagttatattaatttggtgttgaatttatgtggt

SEQ ID NO: 14 - DNA, 828 bp, Glycine max
  CDS (2)..(604)
attcggctcgagtatggcatctgcaacacgtttagtgcactgcgagctgcggtcggcgaggcccgcggttcgggcgagggaaccggccggtccggttcaggttacgatcccgaaacccaaagcggcggaggcggaaggcgcgaacatcgttttgcagccgcggttgtgcactctgagatcctacggttcggatcgagcgggggttctgatcaaggcccgcaaggagggtgacgatgatgacgtgtcccccttcttcgccgctctttccgactatattgagagctctaagaaaagtcatgattttgagatcatctctggtcgtctagctatgatggtgtttgcagcgacggtgacaatggaaatggtgacaggaaactctatgttcagaaagatggacattgaaggaatcacagaggctggtggggtgtgtttgggtgcagtaacttgtgcagcactctttgcatggttctccagtgctcgaaacagagttggtcgaatcttcaccgtcagctgcaacgcattcatcgattccgtaattgaccaaatcgtagatggtttgttctatgaaggcgacgaccctactgattggcccgatgaaccgtgatcaaatataccattcttaccattttacacactttgcagatttaatgtgtctaaaaaccatagacacagttagactaagatgtaatattaaataatatttagaaatatataagcagaggaagaatactaaaagtgatgtcatcccgatgcagaagaaaaaatagtaaaataaataaatgatgcgcaccatagatttgttgaacctaaatggattatgtaaagtgt

SEQ ID NO: 15 - DNA, 1425 bp, Zea mays
  CDS (1)..(858)
ccacgcgtccggtgccccactacactgaagagaatatgcaggcagcatggaattaatcgttggccatcacggaagattaagaaagttgggcactccctgaagaaattgcaaatggtgatcgattcagtacatggtagcgaaggaacggttcagctcagctcgctctatgaaaactttaccaagaccacatggtcagaaagagagttacaaggggatgccacttatccattgtcagaggaaaaaggtcccttggaaccttctgttcctgatcggtattgcgagggcagattcacctcgcatacttctggttctaattccctctcaccctcttgtagccaaagctcaaattccagccatggttgttccagtggttcgaaatcacaacaacatgtcagtgctcctcaacttgcagtaaagaaagaagttttcatggaggagaatcagagctccacactactcaaagctgcaagccatgctgaactgcagatgcttcctgaagaaagacttgtcaccctgcctaggtctcatagccaagtgcttttaagtgaacaaaagccagtggaaaatataacaggtatgcaaatgtctaagcctgattctctcaagataaaagccatgtacggcgaagaaagatgtatattccgactgcagcctagttggggatttgaaaagctaaaagaagaaattctaaagcggtttggcattgctcgggaggtgtatgtggacctcaagtatttggatgatgaatctgagtgggttcttctaacatgcaacgcagacctgcttgagtgtattgatgtgtacaagtcatcaagtactcaaacagtaaggatcttggtacattctagtgaccagtaagtgcctgttcctttggtcaaactggtttgtcctgacaatggcgcaaagctcatcaggatgctgaagaaagcatacaatctctctttgaagtaaagtgcttgttgacatcgcgctcagtttcaagtcataatcatatatgcaaaagcagcatctgtaaatacctacactcgtgttgtcatcccgtggtcaagtgcctagtaagttctgtatgttgctgtttggtggctgtgtggcagaattggagagaaagtgattgttggtgttccctgtcagctcttgggattggtttgtatgcagtaagctgcatacagtctttgatataacagaagatgggacttctgtacctctagttcagtttgttaacagtacaggtagttcgttgaatgtaacaaactagtgtacaatattgtaggccggagggtggaatcagtaggaatttgtcgatatgaagctttggattaggttaagtaatcacgaccaagttgcatcgcgcttgtaagttcttttggattaggttttgaaattcaaaaactcgccaaaaaaccacaagctgtggaaagtggaatc

SEQ ID NO: 16 - DNA, 1308 bp, Zea mays
  CDS (640)..(987)
ccacgcgtccgcagccgcctcctgacggcaccagcaccggcggcggcgacgcggcgaaggcgatggcggcggcggaggacgagtcgtcggcctcgcactccagctccaaggccagcagggggtggtcggcgagggacgagagggccatccacctcgtcccgctgctcaccttcctctgcttcctcctgctcttcctctgctcccacgacccgtcccccgccgatatgtcgagcttcgctggaggcggcggcggaggaggaggagcgagatctgggaaccggaggttaaggatgctttagtgtgcgtattacctacccattggagtattattatccgcgatgcggatgttgtacttgtccagcagtaaccaagtatgggaccaaagtatgtcaccatacaaaacactcttgctgtggtaatactaccaacctacatcagcatgttcctgattaccatttgagctggattcgaaatacctggcaaaaaaaaaaaaaaaaaaagggcggccgctcgcgattttcagcgcctccaggctccggccaactcgcagccagtctccacagcgcaacgaacctcgagtcctcgacaagcacccaaatcgacgaattcttcccccctctccgatctcgacgcgcaccgatgcgggcgatgtcgtcggcggttaatggtatgctgcgggcacggctgcgcggggcagcgcgcgttcgcggcggcggcggcgagggggccgggcggtggacgaccccaggtcacgaggagcgccccaaggggtacctcttcaaccggccgccgccgcctccgggggaatctcgcaagtgggaggattgggagctgccctgctacgtgacctccttcctcaccgtcgtcatcctcggcgtcggcctcaacgcgaagcccgaccttacaatcgagacgtgggcgcaccagaaggcgctcgagcgcctccagcagcaggagctcgccgctgccgacacccaggccgagtgatccgcgcgtctcaggtcctctcgaactctcggctgcctctgatttggttccaataagatggccacaatgtgcggtcctgaatgttctggacttctggtattgatggaacacgagctgaacactatgctgctgttttttctgttctgttttgaaatttgcccggtgatgatggcgaatgtcctgaatattctgaatattttgaaatgtttgactgttgtattatcatcttatccttccgttgtactaccgcatgagcacaatttaggattttacttgttaaaaaaaaaaaaaaaaaaaaaaaaaaaaaaaaaaaaaaaaagg

SEQ ID NO: 17 - DNA, 724 bp, Glycine max
  CDS (1)..(507)
attcggctcgagcagaggcctgttgaaggaaatgcaacaatgaaacaggatttaagaagctttaagcttatattggaatatattaaagccttgcctactgggcaagaaaccgattttgtattagtttcatgttctggactaggaattgagccttccaggcgggagcaggttcttaaagccaagagggctggtgaagattccttaagaagatctggccttggatacacaatagtccgtcctggtccattgcaggaagaacctggagggcagcgtgctctaatatttgatcaaggaaacagaatatcacagggcatcagctgtgctgatgtagctgatatatgtgtgaaggcactacatgatacaactgcaagaaacaaaagctttgatgtatgttacgagtatattgctgaggatggaagggagctttatgagctggttgcacacttgcccgacaaagcaaataactacttgacaccagcactctctgtcttagagaagaatacctgatcatcccttgttgagacaaaatacttcccagaagatttcataaatttgctacctacgttcttctacttctgcattggatcaatgcagaagtgattcatgtaactatttttcctgtatatatattcttgtgtagcacaaatctacgacttgagaaattaatttatttataaattcttcctttgaaaattggaattatacaaaaaatatagatttcaat

SEQ ID NO: 18 - DNA, 2523 bp, Zea mays
  CDS (219)..(2390)
ccacgcgtccggggaatcatggtgaggcttctccgggtttcctctctttctctatctcttcctacatcgccactcgccagtgcggcggtgacctctagaatccaaagcaagctgtagtgagcagccccttacatactctctgcggactgcggtccgcaccgcacgccatctcagcaagacgagagctccaccgcgctgttcacttcttctgacgaaggatggcgggaaaagaggatgagaatgagaagccatcgttggtggcagccggcggcaagcaagatcggacagcggcgaccacggaatcccttccgcaaaggacgaatcttgagtggggaaaggcagcgtgtagcgaggatgacatccagaagtgtgtagctgccggtgccttccatcccggtgagctggtcgaatggcgagctcccgtcaaggatgagactccgacgctatccaccatggaggatcagtttgttatcctgtctctgacgcacataatttgcggtctgagggtcgatgcgagcgatttcctggtcagtgtgctcgagtactacagacttgagtggtctcacctgacgcccaactctattacggcgctgagcatcttcgcccacctttgtgaggcctacgtggaggcgcctccaactgtggaggtcttcacgcacttctacagcctctatcacaataggaaaggcgaaacgacaacactgggcgccgtctacttccggctcagggacaggatgaagaagaattatccattgtactacttgaggtcctcgcagttcatgtgggtttctctgtggttctatgccaaggtaccaaagagctgtcgcttgaccttcaggggtgatatactaaaggaggaaaacaattggaattggaaagatcttttgcctctttcctgtgagcagatgaagcaggtcggccaaatcatgaagctaagtaaccaaggcttgactggtgcagacatcattcatgattacctcaagcgccggattagccctttgcgccgaaggatgcatttgacatgcaattattctggcctctcagatcctaccagggattcagacaaagatctttctgtggaagacattgagagcaagctgagctaccttctagatcttaagaggatgggtgtgaagcagcctacaggtagactggtcagagcatcaaccaatgaccaagccaatcagcctctagacttgctgaatgtttgctcaactcacgaagctaagaaggaagcacaacctcaagtttgtgcctccctacgaagatacacaaggcaatctgctggtcccaggaaggttgcagtacctccacctcttaaaattgaccctccccccactcaaggtccggccccagaagagattctagatgccacaacaaacatagcgactgcagtttctccaaacctgggcgtagaacaaaaatctatagagcaccctactgtggctgaagagcggaaaatagcagagttagtgaaacctacgttttcagtcattggtgccaaaaggaaggcatctgccccccggtctcgttcaaagagaagagcaaagtattcattactttccgttgttacaaagaccaggatgtcatctttggacactggttctataaaagggacttctagaacaaaagaggcagtgttagcgctgaattcacggtcaataggattggctcgatgccctcctgcctctttagcagaagggacagggaacggtggactgttcatgcttgccaaagttgttgaccatactaaggttgttgaggattccatgtcgaatatactccttaatcaacaagttggagattcttctcgcaaggaggttgatcctgcacaatctatcactgagattgtccaagaccaggaggccattgaggtttcagctgccattccggtgcctaataaggaggaaataaacagtggggtgatatgggagcggatgcagaaagttcagagtgaatacgtgtcactcagtatgacagcctcctctgagcttttagaacaagcaaagaagctggtcatggagaataaacgcctgaaggacgtgcagataatgctgtcgcaacaagtgaaagacctcgaagacggcagaaggctcttaacagaaaggatgaagaaagccgagcaggagacattcaaaataatagaagaaaatatgaagctcaaagatgagaacaaagggcagaagcagatgatcgaggagctgagtaagcagaatgagtcaaccctaggggccttggtccataaatgcactctgttggaccgctacaaagaggagtctgctcagctcattagagagaaggaagagctgcaatcgcgtgtttcgcgtgtcaatgatcttgtcaagctagtctctagtactttgtgccaagagaaagacagcgcttcctaaattttatgtagcatttgtgtgtttgacaagtgcaaatgagatccacagaaggaatgtgattggaagcgaatgcagattactgatgtatagaaacatccttatcaatgattgatgtagtctaaaatctcaactg

SEQ ID NO: 19 - DNA, 1528 bp, Glycine max
  CDS (223)..(1251)
attcggctcgagtcgttttggggtgtgcgactattgtcccttctcttcgtttcgtcggttactaaaatacactaattgcctctctctttccctttcttcttcttctccgtcttcttcacactgagttgactgagtcttcgtaactcgctctctactgtctcagctactcatcgcattctctgcatttatcctccgatcacattttctctctcttcgttcgggatgtcggagaaagctcttagggatctcaatacaattcttggaactgaaaggaagaatgaggactccagtaaggcatgtttatccaagccttctgttgacaatgctgttgaaaatattgaagaatggcaaaagaaaaataatagtccctccttggtttctccagctgttaatggaaatctggctgtgactgctaattctggtgcggaggttgtaaatccagaagtagaatatattgaatctgagaacttgaatgatgtagacgatattgatacttgtctcaagaccctattagctggacttgactcaaaggattgggtcctggtttgtgacacactgaataacgtacgtcgattgtctatatttcataaggaagcaatgcttgatatgctgggggatgtgatcacatctattgcaaagtcactgaaaagtcctagaagtgctgtttgcaaaactgccattatgacatctgcagacattttcagtgcatataatgatcttataatagattcattggaccctctgctagtacaacttcttctcaagtcttcacaagacaaacgctttgtatgtgaggcagctgaaaaagccttgatatcaatgactatttggatttcccctatttctttattgccaaaattgcaaccataccttaagaacaagaatcctcgtatccgtgcaaaggcatcaatgtgcttttctcggagtgttcctcagctgggtgcagaaggcataaagacatatgggattgacaaattgatccaagtagctgcatctcagttgagtgaccagctcccagagtccagggaagcggcccgaactctacttctcgagcttcaaaatgtgtatgagaaatctcatgatctcatcaagccagctactcctactgtcaataatgagcatactgtgaatgaggagaatccagaggtgagttcttgggaaagcttctgtcagtcaaaactttctcctcttagtgctcaagctgtacttcgtgtaaccaccagtatcgctcgggagggtcttgtttcatgacacgcacattaaacctcactttgcggctttcatcatttccttgtttgcttatcattttcttctgtaaatacagtgcgacaattgagatgattgagttgagttgtcgtcgaagcacgtattgagcccaagatttataatgtgtattctattattttccccctagctgttgttacctatattacttttgattaatactactactactactagataatggaccgggaaaaattatgagtataatctttggtcttgtaactgtttttggatgattatggtt

SEQ ID NO: 20 - DNA, 1650 bp, Zea mays
  CDS (1)..(1530)
ccacgcgtccgggcattattagcatcaactattgttccacatccgaaccaaggaaatatgcatgaacctgctatcgacatgccttttggaagtgtacttctgcaagcactggtttcaagtgatgtcaatggagatatggaggcatgttgcagagcatctagcgttctttctcacattgtcaaggacaacatgcaaagcaaggatcgtgtattgcaaattcagcttgagacacttacaccgtccttggggcgcactgagccagtattgcatcgtattgtcacatgtttgtccatcgcagcctcaacagagggagaaaacaaccaaaacaaccaaccagaagaaccgtacattcaacctgttattcttcggctactcatcatatggcttgttgattgttcaaatgctgttaactgcctgttggaatcagcagtgcatctgaactacataatagagcttgcttcaagcaaacgttacactgcttgtgttcgtggactggctgctgttgttctaggtgcttgcatcctgtacaatgctagccatgagaagggccgtgatgcttttgctgttgcagatgctataagtcaaaagattggcctcacaacgtacttcttgaggtttgacgaattgaggagaagcttggcacatcctttgccagagcagcatcatcgcaaggagctttcccgatcaagtgcgaatagcatgtcagatttccaagaaattgaagaggatgaaacaaataaagatgatcaacaccctgtcctctcagaaatatttgattcacagtttgttaattttcttagtaagctcgaggctgatattagagaaaacataatggatatatttagtcggacaaaaactgcaactgcactccttcctactgaattggagcagaagaacggggaggttgatggagagtatatcaaacgactaaagtcgtttgtagagaaacagtgcaatgagatgcaggacttgctggctcgaaacgcaatgttagcagaagagttagtgagaactggtggtggtaccaccacagatacttcacagagaccgaacaacgggcgagaaagggtccagatcgaagccctgagacaagaactggagggagcgagacgacagatagaggcactcgaaactgacaagtcccagatcgaagccgaagctaacaaccaacgaaaccttgcagtaaaactggagtccgatctcaagagcttgtcagaagcttacaacagcatagagcaggccaactaccgcctggatgccgaggtaaaaaccttgcggcagggaggcagtgtgccgtatcctgacgtggaggcaataaaggcgcaagccaaggaagaggctgagaaggacagtgaggcggagctgaacggtctgcttgtctgcctcggtcaggaacaaactaaggttgagaagctgagcacaaggctggcagagctcggcgaagatgtggatgcccttctgcaaggtattggtgacgacactgccattccggacgatgatgacgacgacgatgaagatagtgaagagtgatatgtttatgtaaccctgtaggtctgttggtgcacacaacctcacgttgggtcatgatattgtaatggcaattggcattgtacagtggataatgtgtagacaaaggcaccgttccaattt

SEQ ID NO: 21 - DNA, 989 bp, Zea mays
  CDS (129)..(434)
ccacgcgtccgtgacaattgaactgttttccatgtatggaaagcaggtaatgttgcttcagtatatcatatgactacatattctcggtattgtgcccattacagtttcatgtattctgtggatcactgcagtttaagattgatccacaagattttcaagattcagaaccagatattcttgcaaattctgcttcatcgataatagaacggatcaaagaaaacagtgatcagtgtgccgcggctctcaggtcgctctgtcgccggaaaaagggcctcactgtcgaggaggcaagcttgattggtgttgacagccttggtattgatgtaagagccttttctggcttggaagttaagactgttcgattttcattcaatgcacaggcgctctctgaacgttcagctgaaaagaagatcaggcgaatgcttttcccccgttaccagcgtaaaaacgtgaaagcctctactgaagatgagtcttaacctatcagctgtttttcctgagaaagcatgcaaacacctggttcaaagacaaggtgtcaagggaatcaacacaatatcatcttatgcgagtttgctaggagatagcgcacaacctgtcgactatgagaatgaccaaaaacatcagagtaataaggtttgccatgcatggtctctaacatgcttccaagaatatagtttggtttctgacagataataaaaaaaaaacgaataaggtgccagttcatgtataaaagatgaactgcgactttatgctggcggaaactgattagctgaccaagccttgtcagctacaaattcccagtaataataacgagctgcgaggtgaaagggcagagtaataaggtctcgttggcctaaatcagttcttagtttcttacccacaacctgtaactaactttgttgtctcttttcatgtagacataaatgtcatgcaacgcaactgcgattatggcattgttttgttgaactatacataaaagaaaacttgat

SEQ ID NO: 22 - DNA, 1921 bp, Zea mays
  CDS (1)..(1764)
ccacgcgtccgctcagtactgaaaaccaagccttccccacgaattctcacagaagcagctccttggaggcagcaggaaagaagtgccaccaatatctgtcgagaagctgaagggaggccaagaattgcatctgtctatgctgatatagagagaagggttgggggcttcgactttttagagtgtaataacaaggacttcagggctctcagaatattgggtgcattgaacgcgagagatgctaagaacaagaatgacagcagtggcagaccaatggctactcacagaacaggatatgacctaaccacctctggaagcttccaggctcctattgtggtcatgaagcctgcaggaaccactgaaaagcatggggtttcacttgcttctgttgcccccatagcaggcctaagaagccttagaaagttgccagctagatattcatctttcactggcacaaatgagaccagcacaaatgagaatattcatcttcggatgtcaagagctcaattgaagtctgaagaaactgtcagcagtgccaactcgccaaggcctacaagctcatcaagtcccagaaatgtgctaaagaatgcagagcctgagaggagatcccgtccacctgtttcaccaaaatctccaagcaagaagtccaatgaagttgtctccccaaaaggaagaacaagatcaaagccttctcaggtgaaaagccaccgtgatgaggtcttacagagtacagggaacagaataagcttagctaagcaggtagatgtcagcattatagactgtccaaagcttccgggtggcaactcaaccttcgttccaccaagcaatgctgctgcaacagcaagtcataaggctccttcaattctggattcagaccaaaacattcattcactggacaacattccaagccctgtctctgtccttgatacgtccttctatcataaaaggatctcagattcattcaaagatggtgagacacattcttcagaggaatgctggaacccaaacagcctgcctgacacaccacagtcaaaagcgagcagtgaagccaaccagattaaaccagaaaatttggaagttctgatccagaagcttgagcaactacaatcaatgaacgaagaagacgcaagtatcaaagaagtcatggcatcagtcactgcaaataaagatcaccagtatatctatgagatactgttggcatctggtcttttacacaaagaacatagtatcacggcattacctgctcaactccaaccatcaaattatccaatcaatccggagctcttcctcattcttgagcaaacaaaaccagacttagtttttgcattccaaactgtcagtggaactaagaaaagttgtaagccttacacggggaagcttcaccgaagacttgtgtttgacctggtaaacgaaacaatagctcaaaagatgatcatctgcagatctggaagccagccagtaaagtttcttcaatcaaggaagttgagtgggtggcaactattcaaggatttgtgcactgaggttgacaggcaaataaaatgcaccggggaggaggagaatggaaacatgatattagatgaggatacagtcaatggaacgaaagattggatgagcttcgacactatgctacatggcatggtttgggagattgaacgatccatcttcaagggtcttatcgacgaggttatcggcggtgagactatagagaagatgcaatttgggcaaaggaaactgcagaggcagctttctttcagtagtataaactgaatacgcgtcgtagtagtcatttccatggagaagcttataacaactagtaaatgtgacagtttctttttttttcttctgaagactaaactgtacaagtataaatgatcctcaacactagtcttgctgcctgactattgcaattcgagagttgtgcgac

SEQ ID NO: 23 - DNA, 1023 bp, Zea mays
  CDS (1)..(732)
ccacgcgtccgaatattcttggcatggtggctgacacggaaagctcagattcactgcctggcagttcaaatgctgcttctgagatgcctgctaatggatcttgatcgacaaaacactggaaaggctacagtgctaggtgatgctgcgcgagtactgcgagatctaatcactcaagtggaatctctcaggcaggaacaatctgctcttgtatcggagcgccaatatgtcagttccgagaagaatgagctgcaagaggagaacagttcgctcaagtcccaaatatcggaactacaaaccgagctctgcgcaaggatgaggagcagcagcctgagccaaaccagcatcgggatgtcggatccggcaactcaccagcagatgcagatgtggagcagcattccccacttaagctccgtggccatggcggcgcgcccagcaagtgcagcgtccccgttgcacggccaggagggctactctgctgacgccggtcaagcgggctacgcgccgcagccgcaacctcgggagctgcagctctttccggggtcatcggcatcgtcttcaccggagcgtgaacgttcttcccggctcggaagcggccaggccacgcggccgagcctgacagattccttgccgggtcagctctgcctgagcctcctacagccatctcaggaagcaagcggcggcggcggcggcggcgtcatgtcgcgcagcagagaggaacggcgggacgggtagccaactgacagacaacgcacttgaatcggggtgtgtggcggcggcgattgtcctgtaaataccaagaattccgtccctttactggctcgttcatccagaactaatgctggtagctgccctgtgttgagcggcagctctgcttgttctagaagcttctgagaacatgtgtgggctatgttttccatgagattgtaagatgtgaggtggagccgctagaacacttagcgtgcgttttactgacagtgtggagtgctgctccttagttggaatagaaaaatgcaatttgatggc

SEQ ID NO: 24 - DNA, 1599 bp, Zea mays
  CDS (3)..(1274)
ccacgcgtccggccatccgggagctttggaggccgaatcccagtcaattaattctcctacaaacaaggggaattggtgctttgcataaagagcttccaaaagcgtgtgctttgactggcagcagtgatccgtgctacatcgaagcatatcatttggcagatccaactgatggcagaattaccctacatctaaagattttaaatttgactgagctggaactcaacagggtggacatccgtgttggcctgtctggagcactgtattatatggatggtttctctcgcactgttcgtcaccttcggaatcttgtttcccaggatccagtccaaagtagtgtgactgttggagtttcacattttgagagatgctcgctctgggttcaagttttgtattacccgttttatggaagtagtggatcaacagactatgaaggagattatgcagaagaggattcacagatgatgaggcagaagcgctcacatcggcctgaacttggggaaccggttgttttgcggtgccaaccatacaagtttcctctcgctgagctcctcctaccattagagtgttctccagttgagtatttccggctatggcctagcctaccagccatggtggagtgcactggcacatacacatatgaaggcagtggtttcaaggccaccgctgctcagcagtatgacagctctcccttcctcagtggattgaagtcgatttcttctaagcccttccatcaagtctgctcacatttcatccggacagtagctggattccagttatgttatgcagcaaagacatggtttggtggatttgtgggcatgatgatatttggggcaagtgaagtcagccggaatgttgatttgggtgatgagaccaccacaatgatctgcaaattcgtcatgcgcgcatccgatgaatccatcacgagagagatcaaatccgacctccagggctggctggatgatatcaccgacggcgctgtggaatacatgcctgaggatgaggtgaagagtgtggccgccgagcagctgaagatctccatggagaggattgccctgctcaaggcagccaggccaaaggtcccacccgcaaagaccgatcaagaggaggaagaggagcgaaagcagagcgaggaactagatgggtttggaaaccccaagggcccctcgacgctctccaagctcaccgcggaggaggctgagcaccgcgctctccaggctgccgtgcttcaggagtggcaccagctatgcaaagagagagccatgaaagcgcagtgaggtcgatttggaccgtgtggatcttacgcgcatgtataattttgtatttttccttcgtctcccattctttgcgtgtctatactgctggtttcactcactgtataccgtcgccgatttgttcattgtattctcaaatacttgtgcacgaaaaaacctgttttgtaacatactactgttagggcgtttttaagcttcggtgtgaagatcgattgtacaagagaatccaaatgtcattgttgccttcccagttttatgaaaatgcattctgtaattgaagaattctccccaaaatgccacgatttataaataagaaatgtggaattgt

SEQ ID NO: 25 - DNA, 1629 bp, Zea mays
  CDS (1)..(1116)
ccacgcgtccgtgcttctggtattagtggaacttctgtgcgacttactgctggagctggtctccctgttcacatgaaaggtgaactaaacacagctttcataggactgggggatgacggtggatatggtggtggctgggttcctttggcagctcttaaaaaggtgctgagagggatcctaaagtacctcggagttctatggttgtttgcacaattgcctgaacttttgaaagaaatactaggatcaatcttaaaggacaatgaaggtgctctcttgaatttggatcaagagcagcctgcactccggttctatgtcggaggatatgtatttgcagtaagtgtccatcgggttcaactgcttctacaagttttgagtgtgaaaagatttcaccaccagcaacagcagcagcaggctcaaagcaatgctcaggaagagctagcagcagttgaaatcaatgaaatatgtgactactttagcagacgtgttgcctccgaaccatatgatgcttctagagttgcttcttttatcactttgcttacattgccaattttggttctgcgcgagtttttaaagctgattacatggaagaaaggtctttccccggttcatggggacattgctactgcacagagggctcgcatcgagctttgtttggagaatcactcaggatcagcttcgtctgataacaccgagaacagcagtttagccaagagtaatattcatcatgatagagcccacagttcagtagaatttgccctgacatttgtacttgaccatgctcttattcctcacatgaatgtagctggaggagctgcctggcttccatattgtgtatctgtgaaacttcgctattcttttggggacaacaatcatatagctttccttgctatgaatgggagccatggtgggagagcctgctggttgcagtttgaggagtgggaaagatgtaagcagaaggtttcaagagctgtggaaactgtgaatgggtctggtgtagctggagaggtaggccaaggaaggctgcgaatggttgcagagatgatccaaaagcagcttcaactctgtctacaacagctaagagatgacccactttctgcaggctctactgcatcatgaacttgcttggaatgagctctatcaaggccattgatgtctgtcatattcagcatggcctttggctttgttaacgtgtagatgtgaaggaatacacatttggtagcagtgacagtttcgcctgaggcttgctcagatgacaattttgtacagtataaatggtggcggttgactctgatggacctttagttagcccatttgaacgtggcctgtaacatagctagggtgggtaataatgtgccaaggtggaattgctcagggatgcaaaatgatcttcgggaattggttgtctgtatcatctactgtaatcgtgtcccttttctccctttctgatggatagttcatttttggattcgttatcatctttcttctgtctgtactatgttaatctagacattggtggatctggtatatgtgttgttgtacatgatatattggcactggacttgagactgacgcatttgacgtcttagtattagcaggacatctctagttgtgttcagagtgattggaccc

SEQ ID NO: 26 - DNA, 966 bp, Zea mays
  CDS (3)..(539)
ccacgcgtccggatgatccctgtccatatcttctttccatatggaccccaggtgaaactgcacaatcgatcgatgcccccaagacattctgtgattcaggggagacgggtagactatgtggaagttcaacatgctttagttgcaacaatatacgagaaatgcaggctcagaaagtcagaggaacacttttgataccatgccgaacagcaatgagaggaagcttcccacttaatgggacgtattttcaagttaatgaggtatttgctgaccattgctcaagtcaaaatccaattgatgtcccacgaagttggatttgggacctcccaagacgaactgtttactttggaacctcagttcctacaatattcagaggtttaacgactgaagagatacaacgatgcttttggagaggatttgtttgcgtgaggggctttgataggacagtgagggcaccaaggcccctttatgcaaggttgcattttcctgtcagcaaggttgttagaggcaaaaagcctggagcagcaagagcagaagaataatagtacattgaagaaatataggggtgctaaccagacgaggatggatagcccgaaatgagatgctgacccaataggtcgccaaatcacctccaaattctaacccaatgacttccatctgtaatgaatggcaataccttgaaaacctgtgatggagatgttttgtggcgacatgatctcttaaattagattccgtctttggtaacagcctagctgttcttgttgagtcgcatattctttattctgaagatcaatatagcaaatgggagaaaaaacatagtgcacagtcgagctgttctaattgtacctcttgtgagtggaatcagttgttgtacaacaggaagatggacctatctgtacaatggtcgtacatgttctttgatcatagtataaacaatgtaagcctcatcagccgttatttgattgtttc

SEQ ID NO: 27 - DNA, 863 bp, Zea mays
  CDS (3)..(671)
ccacgcgtccgctcagggtgaagaagctccagcaggaggccgcccggtgcctgagcttgcacaagacaatggagctgcagccggagctgtcgctcggccctgtgtggccgggcttcgccgccggtgacctggccgccaagagctcctcgtccgagtctgacggaacctcccggaagaagaggaagcactacaccgccagctgggaggagcctcagcagccgcccgcgagcctggagctccagctcaacgacccactgcctctcgactgggagcagtgcctcgacctccaatctgggaggatgtattacctcaaccggaagacgctgaagaagagctgggtcagaccccaggtgcagagcgtgaacctggacctcaacatctccacggctgccgccatcgacaactgtgccgccaacggtgctgctgccgctgcttccgacgacgaggacgagccaaggaaacccgctggcactttgttctctggaggcagcatggtggccgtgccgtgcgccaactgccatctcctagtcatgctgtgcaagtcctccccgtcctgccccaactgcaagttcgtgcagcctctggcacctgctgtgccgcctgcggcggtggcccattggaggatcgatgccgccgtcaagccgctggagaccctgagccttctccattaggcacatgcacttcaccggcagcagattgtagaaaggaaatcgtagttacgtagtagtggtgcatgggttagtattactattacctgggtaatgcatgagcggtcgaatgaaaattggaaatgtgaaactaccgtgcatgcatggtgacatgacaaaatacaaaatggtgaagccagcctcaaattaaactgc

SEQ ID NO: 28 - DNA, 1438 bp, Zea mays
  CDS (241)..(1152)
ccacgcgtccgcggacgcgtgggctccagcgtgcgcggcgtcccgatcgcttcgtaaccgcctgattgatccgcctgtcctccgaggctttcaccgcgcggctttaatcccgattgggttggctggaccgcaacccaattgggggttaattgggattgctactccgcggctgcctgtccctcgtgttcctggattggcgaggccaatcgactggtttgctgcagcctcctgggtagaatcatgaagctaaaaaatagcgcagtggagacttttaaggagaacaatatgattttcacttctgaaggaaatctccactctaaaaaaatgcgagaagattatgttgctagtccaaaccaaccgggtgctgtccagacaagatgcaaatggataattggagatgtaactgaggtctttgatcgcagcacatggaagcttggaaagatcttaaagatgctaaagaacaactactttgttatcaggcttgctgattgcatccagctgaaagagttccacatatctagtttgagaatcccacgtggtctggaagctcctcaaagcaagccctttcatgcagcagataaggccaccggacgcggtaatcgtcgacctgctgatggcgccttgcccggcgcaagggctgcggatcaaatgggtcaccgagcctacgagctggggagcagcggcaagaagcggaaagcaaccgcagacgcctctcaccatctaggcagagcagcagcagcacactcccggaaggtcgccgcagcctccaacccgaacggcggcagctacccgcacagctcttcacaggccatagaagacgccgaatgctcggtggccagctgcagcgtggacgacctttaccgcctcggcaatggcggcaacgccaagcgtcgccccgccgccgcagggtgcctccctgacgacgccatgtccgcgtgcccgtgcacgcccggggcgagggacggggaggacgacgacgcggcgggcgtgcacgggctggagctggaggcgtacgggtcgactatgcgggcgctgtacgcgtcggggccgctgacgtgggagcaggaggcgctgctgacgaacctgcgcctgtcgctcaacatctccaacgaggagcatctgctccagctcaggcgcctactgtcctcgtgattattggcacttggcattggcgcattgccagccaccacccttctggccttctctttcgtacgtctccagcgtcgtcgtcggccaacggccatgcacgtcggcttggctcgcggaacgccgcgttcagcgagcctcttgcttttactcttgttgctaattgctattacatggttctctgtctagtctaggtagaaagaatcctatgttcataccagtgtcgagacatgtcatgccttttgtatcctggatatagtcttgcttgtaaatgcaaacaagttttcctttg

SEQ ID NO: 29 - DNA, 1146 bp, Zea mays
  CDS (1)..(1011)
ccacgcgtccgccgacacgcacggcacgagcacgacagatcgggcgactggagtcatcgccgccgggccggtcgcggtcggatccccccgctccgcctcctctccttcgattcgttgcttcccgcttcaaatccaagccaccaccctttccttcccatggcttccgacgccccggcggagcaaccggcgacgcagcagaagcccaccagggtctcgctgtcctatgaggagatctccaagctcttctccctccccatcgctgaggcggcctccatcctcggagtctgcaccagtgtcttgaagaggatctgccgcacccacgggattgttaggtggccatatcgtaagcttgtttctgggaaagctggggatgacacaaaaggtcctgatagcgacaaagccaacgaactccttgaggtatcaaaaatcgcgaaacaaaaggctcccagtgcatcaggcccatcagtagtgtcatcaagcacttcccaaggagcggcaaagtctcaacagggcaattctaaggcaggacagttttcagtttcgccaccaacaggtaaacataacgcatcactaagtttgacccatagccaagccaaggccatcccttgctatatggatgatttcaagtacggattcccgtcatctggcttgtcttgtgaaactatgaaatggtggggcacaagcagcgacacagactatgtgcctaccaaagatggaagccatgaaccccatgaatcaacaacacatgagccatcaaagggcatgactgatgatgacgagttggactggggagcagatgaagcggaagctgaagctgatggcactgttacggcagaggcatcggcacagctgtgctcgctgagaaggaaagcagtagatgacgggcgcaaactattgaatggtcacaaccgcaggggccaagaattctctaggctgaacaaaaggcagaagacagcgctagcccaggtatttggggcttcactgccagaatgttgtattactcgtgtctagtgagtgaagaacaactgtagctgtgttttccctccttctccagaccctcatgtaatctgacccgttattagcgagaagtgtgaaatcttctcactgaattgaagaatggcaggagcttgttttgtcttccttatc

SEQ ID NO: 30 - DNA, 592 bp, Zea mays
  CDS (3)..(590)
ttgagatagaccccatcaccaaggaagtcttggcaacgccgatcgccgatgccctggggcgaaaatttacccgctttggacatcaagccaaggaagacaggcaggccgccattttccggtctgagaatgggaacgtatggcaggtaaaaatctttggtgaagacaagacgggcaagcggtcagggcagtatctagcaccgacaggcataggggacgtgccctatctgccaacaattccccgccggatcattttggcgatcgccgaaaagcatggggtcaaaccaccggaagatggtcaagatttttggccctggtttgtggatcatcctgagattcccttgattgtgacggaaggggggaagaaagctttagccgccatcagccaaggttacgtggccctgagtctctatggctgtttatgtggcaacgatggtctaaccatcaaaccgtcattattgccctatgtgcaggggcgagaagtagcgatcgcctatgaccaggatgcaaagggtagcaagggacgaaaggcagtctttaagggcacaaaacggctggctcgtaacctgacctatcacgctaaggcaacggtcaa

SEQ ID NO: 31 - DNA, 831 bp, Zea mays
  CDS (1)..(456)
ccacgcgtccggatgaacctgcggcggcagaccccgctggccgcgatccacgccgcgctggcgtcggcggacgccatggtggccgtgcacggcgcggccgtcacccacttcctcttcatgcgcccgggctccgtgctcctccaggtcgtgcccgtggggctcgactgggcggccgacgccttctacggcaagcccgcgcagcagctcggcctcgagtacctcgagtacaaggtggcgcccgaggagagctcgctggccgccgagtacggcctcgacagcaccgttctgaggaacccctgggtgatcagcagccgcggctggtgggagatgaagaaagtgtacatggaccgccagaacgtcaccgttaacatcaagcggttcggcgagctactcaggacggcgcggacgcacctcaagaacaccacggcgtgcgccgccgccgcggcgctgaggtagaagatgctagttgagcttggtcaggtgctaatgacttagtcatacttgtcaatttctttgtgttttcgtcgagatcctgtaaatatgattgggttggttcttttggtcgagagattggtggggatcgatcgccttcttctacagtgcattcgtttggttcatggtgttgctagagcagaagcagatgaagaatagtttatggcaggggcagctgtcctcaaacaccttgttcacactagttcataggggtagcagctctttttatttctcttcaaattttctttttgttcttctcattctttgggtgtcatgatgatgtataagaaactacatgtctttctctgtaacaaagatcaatagtccagactcttttac

SEQ ID NO: 32 - DNA, 1321 bp, Zea mays
  CDS (3)..(1073)
ccacgcgtccgattgaaccaggatcaaggccagaaacttctgactatcctcagtcaagtgagaggccactgactgccacaagcagtttcagttctgcatccccattttcagaatcaagccagttagcttcatctagtaaacaaccagccccatatctacctcgcaaccatatgggcaggcggtctttcatgtctaaaccagtctacccacttgtcttccggaatcctgtctcagaatcagaagcgtgcaggatgcttgaggttggtaatgccgggcgagcgacaccaagtgatgacagccaagcttctcctctgtggcgtcgcagcttggcgagcccagatctcaagttccacaatgcaccgaatgaacttgggaagatggaaacctcacccgaaccgaacacaagctcaagaagggaagggttcagatggagcaacgccagcagttatgactttggatacgatggagatgccattgacatttcagatcatatcagcatcgagtcccagagatctcccacgagctcagcgaggttcctgaagtgtgggctgtgcgagagattcctgcaccagaaatcaccctggacctcgaacaggattgttcgaaacgccgacatgccagtggcagcagttctgccttgccgacatgtcttccacgcggattgcttggaggaaagcactgccaagacagaagtccatgaaccaccttgccccctgtgcgcgcgagccactgacgatgaagggcacgtgtcgttctcagaacctctgcatgttgccctccgatctgctcgcaggaacctttcgttgggcactggtgctggtgggaacagcggcatttctgaccctcctcgcactgatcgtggcttgaagaggaacaactctgcagtcatgcctaggcgcagcggcggcgcattgttccgcaatcgcttcaagaaacagttccccttcaaagcgaggatcgggaaggagctctttggcggtagggttctcaacaaggttggattgtctttgtcttcaggtcagcatgatgatcatcgacagcaagcgccaaagcatgaccggcccatgaagtagctagatcttagtggcacagagtataatatgttgtgtgccctcctgatattttatctatggcatctgttatcgaaacttggcatccatgcgcagcagtcgtccaagatgggggccctgtatggattatggaaggctgcttgcattgcgtgtaacagtagcaccttgccattgcatgtattttgcatgtagcacagtaagaatctatgccctgtgattgatatgttctgaatgttccgcttgatctgttc

SEQ ID NO: 33 - DNA, 3297 bp, Zea mays
  CDS (117)..(2939)
ccacgcgtccgggcgaggcagggagctttccaggctccgtcgtcccgatgactgcgcagctataacctcgggggcgcggcagcatgcgtcttaacgaaggataaagacctgctaagatggccatggccatggccaggttcctctcgtggttgttcacatgcttcgcagctctcgccgtcctggaggccacggtccctgctcgttcatggcgcgctccgagtcccaccccgaggcacgaagcgaggaggttcgagcagaagacggacaggttctgggagtaccaggagcagagcaatacctgggtacaagtacgcgcgccgttcgacctcatgtcctgcatcaacggcacctgcacaaaggtaggatcgatcgggcggctggcgagggagcctggacgacacggccttcctcccgtccagagccaggaggaggaggaggaagacacgcggcgggttcagggagatggtgcagaagaagaccctgtcctgcctgtaaggaggagaatttccttgacaagaatgtcagagtcgtcagtctgggtgacagggcagagcggctccatctacgagaggttctggaacggggtggtgtgggtgattgctcctcatgagctccctgcctcggctgggtatgccaccgcaactttcattgtcaatacaactatccttgctctgtctgaagctggaaccctctaccagttgcagctaaatgagcatgcccagcctatctggacagagatggcattcaactcaagccagcagtcagcgaatcttggattaaaaacacaaagtcaagctatgcgtataagaaatgggattgtatccaatgatggaaggaaacttttcctgtctatcatgaatggatccctgcttgaggtaacagaaattcagcctctaaggtggaattaccatgggcgtcctccaggtgcagacgtgtcgtatatatctgatgctggaaatctgcggccagggaccctgttcacagtaagttccactggagacctctatgagtttgacaaagaaacaaagccatcatggaaaaagcatatatggagtgaagaactggcgaaaaatatctcattaaaatcatcggctggctttgctttgcatggtttgtcagggtctaactcagtgtctcttttcctgataagcaaggatggccttttagtggagcggcgcttgcatagaaggaagtggaagtgggacaaacatggagctcctacgggtcagagacttagttcaattgcagaagttcagaaggatgaacttaatgatgcgacttcaatgtttttaacgacaaccacaggaaaagtatatgagtatcagtttccaaaatatacaggtggggcccagagcaataagataagaggacaatggataaatcacatgtcccctgagcatgcaaaggttgcaagaaacgtcccaggtgtacatgttcaagtcggcagaatggtattcccactagatgatggtaggcttggggaactacattttcctgggatggggggtactgattttggtccgagtgcccaaagtaccataagaagaaaactatcaaacaagtacgagtggtccatcctagacgcaccagaaacagaaggttggaatgcagaatattgcaccgaagagcacggtccgacaaattgtattagtggagcaaagaatatagctgcagacacagaatcaaatgacttgagcaataacccaccttccaggaggcgtaaagtagaagagaagcagcactacctaaatgttaacagataccagcagagtgatgaaactgaatcatacaactttctatcaaggaccattgatcttaacttccacatgcgggtgatgcatgcagacagatcgcttttccttatagcagacaatggattgacttttgaatatctaaacagcaatggtgtttggttgtggttaagacatgaacatgttacagccatgaaaggaacactaggaagctacaatggcagtttgtatcttgtcgacgtgcatgggaacttacacattagagaaagaaatggagatgaactgttgtggattaactgcacagcgatgaagaagggaagacaggttgcaagtgggtctccatgggatggcatccctggtttattgcgcagagtgacaacagatgatgcactcttctttgtcaacaagcgaggcaggctgctacagttcacggttgcgctgcggaaattcaagtggaaggactgccacagtcctcctgacaccaagattgctttcatagtggaccaggaggtcttcagaagaaacatcatcttcgtggtaggccgcaacggtcgcctctaccagtacaacaggatcacggagctctggcacaggcactaccaatcacctcacctgttcctatcgtgctccccagggacggccatgcggccatcgccgctgtccctggccggctccctgttcatggtgtccgagcacggggggctggtggagtaccacttcagcccacaggacgggtgggagtgggtagagcacgggacgccccaccggggcgtgaccctcgtcggcgcccctggcccgtgcttcgacggctcccagctgttcgtggtcggctccgacgggcacgtgtaccggcggcacatggaggggaggacgtggaggtggacgagccacgggcacccgccgtcggagccggccgccgttgacgaacagagctgcgccacaccggacacgggcgccggcgcgcactacgccgacgggttcagggggagctgcgacgggaaggtggcagccgtgcggccggtgccgttctcggaggacgccgtggtcttcgagctgcgagatggccggctggcggagctgcggcggccgccctccgcggacgggtgcggcgggtgggagtgggcgcggattatcggcacgccggccagcgcctgtatgaccagctactggacggccgtcgccacgtagccgccgccgggcgcgactcacggagctggcagtccgctggtcgcttggctgattgaggtctccttggaagttggaaatgatccacaacaacagcggtcacgtccatgtttcctcgaaagacggccacgtccacatgggtctaggccgtatgatggccagatttgggcgcccaaagaaaggcagaaaggaactctctcgaaaaaaaaaacggctaaccgttacattacatttgattgtctgtttggtaatgcatttttagaatactatagttttaaagcatatagtactatagtttacaattgtatataacataagtattacaatattattttaccacagtaaaattatagtatttctt

SEQ ID NO: 34 - DNA, 2134 bp, Zea mays
  CDS (2)..(1672)
ccacgcgtccggcacaatggagattggtcttagaggtccaaccaatctatttggacatccaactgacaagcagatgattgaattggatcaagcattatcacagtggaatactgactttgacaaggttccagtgacaaaaatcgcatttgggcacttcccgttgtctttctcagcattaacagagtcaggaaaaagtatcaaggatgtttttctaaagcaatcattggcagcatatttgtgtggtcatcttcatacaaggttcgggaagaacttgaaacgatactaccatcgggcagtccaggaatcatcattatcagagcattactaccaacataacatgcaccaaggagatgcattccagggtaataaggaaaactgttctgaagaagcttctcatattgaagagttctgggaatgggagatgggtgattggagaaagagcagaagcatgaggatactagcaattgatgatggttatgtttcctataccgatatagatttcagattaggttcaaagagcataatcatactacctacctttccccttgattcaagattcatgcagagagcctctgcttttcgtgattttaaatgtcatgtcatgggggcatcatcttttgataccgtgagagctcttgtattctctaaacatgagataatatctgtttctgtaaaaatatacgactcaaggccaggaactcttgaaatagtctttgactctgaaatgaaaagagtggattccaatgaaactcgaggaaatatgtatttgataccatggaactggagggcatttgaagattcctctcccagccgatattggctccaaattgaagtgatggatataacaggtgacacaagtgtcagccagttgaggccattctctgttaacggcttgccggcaagagtgaactggacatggaaagagttttttgtgattggtattcagtgggcttcaatatatcatcctgcactgtggtgtgccttttctctaatcttttcgttgcttcttgtaccacaagttttagcagtggtattcaaagatcggttcacatataaatctctatgcgcatatggtgggcagaggacactgttgaagtctctagttggtggtttcatctgttgttttgttgaactctccaggctggttcttgtatggttattgctactgttatatgctatctatttagtttttataccttggttattcggtcaccctattactgaagatggtagcctgacttacatgacacataaaggctggattctaaaaggacccagtagtagcaacgaagtagtccatgctgggattccagatgtcatggtcattgttctacctcacctttgctttgtgttagtacccaccattgtgattttagctgccatggctgctgagagaacagcatatcgagagcattatctttctcgatcaggaaagaagaaagatgactaccgtaagagcaggacacagatagaacatgaaaacttttggaacggtcgctggattagcaaatttctgtgtcttctctgcgtggtggttctatgcaaacattggaagctttgcagagcgctcgtgaaggcttatgctatgaaccccttgctccatgcgccagtacttttcttcttcgttcctttactcatggtgtttgccatctacaagacacggtccatttagctgttcgggagttctgaagatgtgctgcactggactgtccaataaatggtgctccaactgtgaagactgaagactaacccacaactgaaggtgtgctacttcgatttctcaatttctctctttcagagagcagatttgttagattgtttaacaggcatatatagctttcagcattgcgctccgtataaatgagggactgcacggcatttcattagaatggaatagttaggatttggaccaaggaggaagagactaaactgctcaatatagtttttcctctttcttttgagaaagttctttttcccagctctgtttttgacctgccggctttgtttttctacatttatccttcttgtgagactttgaccatctacagtataatagcactgtcgcttgcggaatgcgtgtttacactgctccaaaaaaaaaaaaaaaaaaaaaaaaaaaaaaaaaaaaaaaaag

SEQ ID NO: 35 - DNA, 899 bp, Zea mays
  CDS (183)..(686)
ccacgcgtccgaaccgtcgtggtacaatctgtgacaagtgtcctttggcttggcatccataggccaccgccatcagcagtccaatacagtgacctgacctccacgcgaagccaccaccacaactccacaagatctagaacctaagaacctgcagaggagaggaagaggagcttggggaggagatggccggcgccgagggcgagaggtgggtggggctagcaacggacttctcggaggggagccgggcggcgctgcggtgggcggcggccaacctgctgcgagccggcgaccacctgctgctgctgcacgtcatcaaggagcccgactacgagcagagcgaggccatcctctgggaatccaccggctccccgttgattcccctctcggaattctctgaccctatcattgcgaagaaatatggagcaaagccggacatagaaaccttggacatcctgaacactacagctacccagaaggatatcgtggtggttgtgaaagtcctgtggggtgatccgcgtgagaagctctgtcaagttatccatgataccccgctgagctgcttggttataggaagcaggggccttggcaagctcaagagggtgctcttgggaagtgtcagcgactatgttgtgaacaatgcaacctgccctgtcaccgtcgtcaagtcaacaagcaccgaaggttgatcttatcgccgagatgcctggatgtgatgttgtaataaagatggttggcctgatgaagaacatgcactagtttctagctatttgaaacatgttgaacgaggcaattctaatgtaataataatagacaaagtgccttatgaaggacatggttgacatgcctttgttgctgtatgagtgatgtttatagttttgagggtttgaagatgtggatcc

SEQ ID NO: 36 - DNA, 700 bp, Zea mays
  CDS (363)..(605)
ccacgcgtccggttgagatggtttagacatgttcaacggagacctacgtaggcacccaatgcataacgggattcaaagacgtgataacaatgcgaagagagagatgaaaaaaccgaaattgatatttgaagagatagtaaagggaggtttgaaagaatgaaatattaccaaagatttagccctcaatgttttgttgttattgtaccaaagaatggaaaatactgcaggggactaaaatgttttgttgttattgttgttttttgtaagcatagatattgtgctaatgctccagtttgttctactgcttcagagaataaaaatgttgttgcataattgattgccaataggtaaatgaagacatccagacaagagcatatctaagcaatgtctgtgttgccaaggagcttcagaaaaaaggcttaggctatacattagttgacaaatccaagaaattagctcttgaatggggcataacagatctgtatgttcatgtcgccatcaacaacgtagcagggcaaaagctgtacaagaagtgtggatttgtttatgaaggcgaagaacctgcatggaagggtaggtttctggggagaccaaggaggctgcttctttggcttgatatgagcaaagtgcttgtgcgatcgtgcatgtaaatacatatttagctatttgtaatcatattattgcaattgttggttttaat

SEQ ID NO: 37 - DNA, 1481 bp, Zea mays
  CDS (179)..(1054)
ccacgcgtccggtcccacgcacattcgtctgttctccgctagtccgcagcaagtgacttggtctagggatctctcgagcgcttcgagaaagtttcaggcccctgcacgcgtccactgaccagacctggccagcccaggcgtggaggtgctcgtctcctccgccgcgccgggaccggcgatgaacggcgggctccctggattccacaatgcgccggcgtcaaaggccgtggtcgtcgccgcaggtctcttctccgtcgcctttggcttccgcggccactccctcaacctcggccttgcctaccagagtgtttatgaaaagctgagtgtatggagactgatcacctcattttttgctttttcgtcaacccccgagctgatctttggagcagtcctgctgtactactttagagtgttcgaacgacaaataggttctaacaagtatgctgtcttcattatcttctcaaccatggtgtcggtactgcttcagatccttgctttaggttacatgaaagatccttctctaaatcctttgacatcaggaccatatggcctcatctttgcatcttatgtgccattcttctttgacattccaatctcaatgaagtttcgcatatttggactgagcttcagtgataagtcatttgtatatttggcaggactccagcttcttttctcatctggaagacgttccattgtacctggactttctggcatattggctgggcttctgtatcgcctgaatacatttggcgtccgtaggttgaagttcccagagttcgcaacatcgctcttctcgcagttgtcattgcccttttcaagcaatccatatcaagggttaccgatcacagaaaatgatggaagcatcccttctcatcaggcacgtcaaattgaggatgcacgcacagctacccaagatcctacggagtcttctattgccgcactggtgtctatgggctttgatcgcagtgcagcaattcaggcgcttgcattgaccaactacgatgtcaatttggcgtcgaacattctgcttgaagcacaagctctacagcaatgataccctggcatcgagtcacaaaagacgggcagatgctctgtacattaatgagcgtgaagtgtttgatactccgattcattcttctgttctagattttatcacaggtttccttgaaacaagccaccgtatatgaacgtgaactatgggaacgaaagacaattgatgcaggagctttctctatttgcttgcagcgattctgaatggtggttgactagtgaatgtctggcagtgtgcattggcaatagtgttggctttaaagattgccagcagattatggtggtttgtggatatgtagccaagtagccaattggtgtcaggaaagaaaagttgctataatcgagaaagcgaaaggttgatgtcaaacagctcgcactgattttcctccagttcccccctttgtggcagagcgagaacttttttcattttt

SEQ ID NO: 38 - DNA, 1600 bp, Glycine max
  CDS (281)..(1165)
attcggctcgagcctatatgttctcccgagtctccctctaatactttctcttctctttctctcctaattggtcgagagagaaaaagagataggtagagaaaaacgaagagtctctctcttctttatgtcagggcacaaatctttcacctcttcttctttcttctgacccatttcagaaggaaccaccaaaaaatagaggcagaaggttctagaagccgccaattcttttttttttttttggtttaatctgtctgagtccgagaggtttgtgattgtaacaatggattcctcgtgtgtcccaaacggtgatgtttccgggttcaaagataaggagccaatggttgaccctttcttggtcgaggctcttcagaaccctcgtcaccgtgtcaccattttgcggatggagctggatatccagaggttcctgaataatgcagatcagcagcattttgagtttcaacatttcccttcttcatatctcagactggctgcacatcgtgttgctcaacactatggtatgcaaacaatggttcaagataatggcttcaatggccagggaaccagaattatggtaagaaagatagcagaaagcaggtatcctgtggtttgcttatctgaaatacctgctaaacagttggaagatgataaacctgagcagataaaaattgccataaggcccaggcaaaataaaaacagtttaaatgaagccggaaggaaaagcaatcctcttagaagcgtggaagagagaaaggaggaatatgatcgggcacgagcacgcatttttagtagctccagaagttgtgactcagatgatactctgtcccagacttttacagatgagaaaaattctcttataatcaaggatgagaatgaaactagcaagacccctgtggttgattcagaacaatgcactgttggcagggatattagttctactcgagttgccattttgagagatagggaaaaggaccgtagtgatccagattatgatcgtaactatggaagatatgctaggagtattccaatttcttcccttaacttgatgccttttaatttgcaacaagttcaacctccatttgtgcagtatgacaatgctttaatccgttcagtcagatatcacaaaatcaagcttcacttggctatggacctcctccaagccctatgatgaatccttttggtgtcacggggtcaaatcaggtgtctagggatggtgcttatgtacagtggccaagtgctgcaatgatgtatgcacattcatatgaccaatttagacatgctgttttccaggctccgattgctcaacgcccattgagtttcgattattcacagaattactagatgacgtttagagggaagttttccatttggctgcagatgttattttagctattaaggatttcagagtaagatacaatttttctgtactgttataatagacccttgttttctattctggtttttaagttttgatggtggtagtaagcattcggtatttcctagtgaaatttttatactggactgtagtgaaagtactggatttgaagtctatgaagagttatatgtggttaccttgtccaatgttaaaaaaaaaaaaaaaaaa

SEQ ID NO: 39 - DNA, 718 bp, Zea mays
  CDS (3)..(716)
gggcccggggatcggcgcattaccggactttctgggtaactgattcccattatttaacagcaaccgggccggcgatcgccatatttaccaacccgaccaaacaaggctatgacgacggactgggcgaaaaaatcattggcacgtttggcaactgtgctgggggaacaaccccctgggggacagtgttaagcgcagaggaaaatttccaaagccaggttccagaagcagtctatgccgatggctccgccgtagatccggcccaatgtccgcttaaaataagtaccaacgggttgagtggccaaggcaatgtgtttggcttggcgggcaataaatatggctggatggtagaaattgacccggccaatgccaacgattacggcgtcaaacatacggcccttggacgctttcgccatgaagctgtggcggtgcgggcaacagcgaatcaacccttggcggtgtattcgggctgtgatcgcactagtgggcatctctacaaatttgtgtcagcagatactgtcaaatcccccaccgataaaggcaattcccgtttatttactgccggaacgctctatggggcgaaatttaacgccgatggtaccggggaatggattgccttaacccctgataccgtagtaaatcctgttcgtcctagcgatattgctgttgatagttctactaccggaattgtctatttgccccatccagaccggaaccaggc

SEQ ID NO: 40 - DNA, 1866 bp, Zea mays
  CDS (1)..(969)
ccacgcgtccgggttcccctccacaggatgtccgaccccgccgctggaggcgccatggttcctgctgccggacgcggcatcgcatgggcgaacggcggcccccggttcggcgacatggtctgggccaaggtgaagtcccatccgtggtggcctggccatatctacagcgtcagcctcaccgacgacgaggaggtacaccgcggccaccgcgacgggctcgtcctcgtcgccttcttcggtgactcgagctatgggtggtttgaccccagcgagctcgtccccttcgaggaccacttcaccgagaaggcggcccagggcggcagctcccgcagcagctttgccgccgccgtggccgaggccgtcgacgaggtagcccgccgcagcgcgctggcgctgctctgcccttgcgacattcctgacgccttccgtccccaccccagcgacggcaacttcttcctggtcgacgtcccggcattcgacaccgacgctgactaccaactcgaccagatccgggccgcacggcagcgattcgtgccgcggaaggcgctcaactacctgctggacgccgccgtgactcagcgagacgcggcggaaaaagccgcgcgcaccgtgcctgggatggagatggccgcgctgttcttggcgtaccgccgcgcggtgttttcgccgatagacaacacctacgcccaagctttcggggtggatcctgagctggctcttgctgccgagcagaaagccgcggcagagagagctcaacgaggtatcaacaacactcacatgctcaattctagctgtactgttgttcttgttactgtgtacttgaaattgatgggtaaacaatgttcctactgcttttatagatcaagcaacaattattttgatgtgaataacggagtggatctgattcaaatagtttctatttcacttctgtttaactgttggttgcattttttttatcaacgagagtgattattgaatttgggatgatattcatgcagaaacttgttgaataaattgtttgtttcacgggattcagtccatctaacctagtaaaatatgagacatgcatattcacagatcaatctgcatttgttctagaaattgaacggctgtgtgatatagtatcatatacgtgtctatacgatactatcctgatatgtatgctgagttaggtcttttctcattctagcttttctctaggaaaaaaaacaccatccatgatcttgttggcatgaaatgtttaaagttgtgactagtttgtatcatgaaggtttaaagccatgactagttagcatgagcctgttattggagtcttgagatgttttatttctatatgtccatacacttacatgactaggaagttttctctgcagtatatttggttcttttgcactatggtgacaattattatacatttggaacttgtgtttccttcctattttagtttttgttgttgcacatcagtttcctgtccatattaagttatgggttcacatataaaagctgcattttttttatcaaattgttaagtttgcttgtcaaggaggatacacctagattagacaagttgatccttacgcatgtagccagtacagagcaatgaaatccatgttttgtaaacagaagtgcgataaactgtgtgaaactgattctgaatgaacgcccatgcatatccaacctagtaaaatattagacatgcatacccacagatcgatctgcacttggtctagaaactgaaggcctttgtgatatagtatcatatatgtgtcattttttctttatgatgccatcctaatatgtatgatgagttagttcttttctcattctagttgtcttaggcaaaaaaaaaaaaaaaaaaaaaaaaag

SEQ ID NO: 41 - DNA, 915 bp, Zea mays
  CDS (2)..(565)
ccacgcgtccgaagcttcagctgctcatttgacaaactatgggaacatggtgagtgctcaggagcgctcaatacaacatactgcttacaaccctgaagttactttgaacttgccaccaccacctccacttccaacaataccacacagttctgctacattacagtctcaaggtggccacagtttgccctcacagacaaaccagcaactgtatcagccagagcagtattatgtgccccaaaacaactatggtccattagttccagttagccattctaaccttcaaatcagcaacactaacaaccccacccttaccatcccacaagtgaaccctggacctccaacaaataatcagattgggaatttggcccagccacagcattctatgccactgcatgttgatagagcaagtcaggatttctcttctcaggggcaacaacaaaatcgtggtcctggtgctgcacaagctcccgaggaggataaaagcaagaagtaccaggcgacactccagttagctcaaaacctactgcttcagttacagcagcgtggatctggaaatcaatcctgagacctaattattgcttcaggtaccctcgtctcgtctcagctggtcgcaaacaactgaaaatatatatcccaactcgacggcttcgcctccactctacaatcgaagcggcccatggtgtgtcagctcgttggcctacacctacttgccccagttctcccttttcaaggacatacattaccagaaaacgatttctttttctcctttttattttatcgtgtatttgatatggataatccggcctgaattttgtactcgtttgttagtatggcctcgtgttatgttgttatttactctgaaaccatatgttattgtgtacggtgccgtggttccaacacaagggaagcttgttc

SEQ ID NO: 42 - DNA, 1360 bp, Zea mays
  CDS (2)..(778)
ccacgcgtccgatcccactgaatttattctggaaactttggaacaaagtgatccacaatcattgatacaatatcttgcttaccaagatttgtgtgtggtatctgagtgtaacttggaaccgtggcgcagagctgctttctttgaggaatctggtgaaacttatagaagaattgtgacagcttgtttgaagccacttgaggagtttacttcaaaaattgctgaagcccttgaaggtttttctagtgatcagccagaattgatgctacaacagtccagactctttagtgcatttgatgattcccagatatgcacatggtgtgctaggacactggctggattaactgcacgctcacgcaaggaggatcgatatggtgttgctcaactgactggctgcaatgccgctgtgatgacaacattgctgtctgccctagtagcaatcgagacatgtttaggaaagaaaacaaacccacagcctgtgcgctcactgggacctgaaaacattaggtggactaacctctccacaggacgaaaaggcaatggagttgcaattgcaagcacacaaaaaagtggcctgcacaagaaagcttatatcatggcagatgttcttcggacttctgtttatcatatactttcagcattcattgatgacctgcaagctaatgcaaaaccatccagcttagagaagaactggatcagtgaagggagaaagcctgtatatggttcacaagccgtgttggttcagaagttgattctgtttattgagtaccgagctgtatgaacttggttccatgtctctcagatcctttgcctgcacatcagatcatacttggatggaagtgttttagttctcgaacttggttgaagaacctttgctttccaccagagcctataaagatgcatcgtaaactaacagtttcaccagagcctataaagatgcatcgtaaactaacagtttctcaatatggacttctggcttctgtattcctgtgggctggtgcactgcggaaggcaccgccgctcaggccttgtttcgagtctggttatccactcaggccttctattctgacttgtactttgaaagttgaatagagttacctgtgttgtagcatggagatggtatgtgaaagatttgcgatttttgatatccacgtaccaaatagatttatgacacagactgttagtactgtctaactacaccttggaacgaggggcatattcaggccctgtttgggaacatagtttttaaaaaccacattattatgatatcatggtgtttcaattgtcgtgctgtaaccatgaattgtaatttcaattgaacacctgtttgtagacagtattttggttagcatggttttggatc

SEQ ID NO: 43 - DNA, 1160 bp, Zea mays
  CDS (2)..(868)
ggggctggaggaagaggacggcgaggaggcggctccggcttctccgtgggcggaggctgacgcgcaagccggtggcgcggaggcacagaccgaggtgctcggcgccggcgagcctgatctcgagagcaagatcgtggccatcagggacttcttggaggaccctaatcagcccgagaacgagctggtgagcttgctgcagaacctggcagacatggatgttacctacaacgcactccaggagactgacatcgggcggcaggttaatggtctgcgcaagcatccttcggccgaggtcaggcggctggtgaagcagctcatcaggaagtggaaggagatagtggacgactgggtgcgcctggacaattccggcggtgacggcagtgcctctgttatgactgatggtgactccccacataaaatccaaggcagaagccaccaaagccctcgggtttcggggtttcagtattctccaagcccacagaggtttaatggttcaacttcagagatggctaacaatgggtttgagtcaacaatggatgcgaagcgtagggccagccctgtaccagcacatcataactccaggcagatgaacaacaatcatcattctactactattaccaccagcacgtcatctgctccagctttctcggtgcagaaagtgacaagggagcaaaagcagagtcttgtggaccttgacaggcttgattctgccaggaagaggctccaggagaattaccaagaagcacaaaatgccaaaaagcagaggacaatccaagtgatggacatcaatgacataccaaagccgaagagcagaaacgctttcatccgcaagagcggcagcggtgggctcccggcgaggcaccgatagcctaacctccttcgtcggctggatatctgcgacacttgcgaagttgtgccctagaaccgcaacgggaaagaaaaagaagcaaacggtttcttgttctttttttccctgtaccttgactgcctaggtttacctccagtgtaatattgtcaggtacataagggtgaaatgatatgatgtttctcgaggaaaaagaaagaaattagtcactgtttttgctatggtttggttgctgttattgctgttggtcagtgctggatataatatgcataaaatgtatcaagcctgcacacat

SEQ ID NO: 44 - protein, 63 aa, Zea mays
FCECIFVVLQQQGPDYMVRNARRSMLEELEGMLEIVEPQPPGKPRTLSRRRFDLPEGVAIEKE

SEQ ID NO: 45 - protein, 473 aa, Zea mays
MAKTPSFAVAAVAGGRGPVHNRTQLLLLLLVAVAASASTAGFLLRGALRDPCDGRGDPAALNTAVASGSPLGFMRSKLVLLVSHELSLSGGPLLLMELAFLLRHVGSQVVWITNQRSQETNDVTYSLEHRMLNHGVQVLPARGQEAVDIALKADLVILNTAVAGKWLDPVLKDHVPKVLPKILWWIHEMRGHYFKVEYVKHLPFVAGAMIDSHTTAEYWNSRTSDRLKIQMPQTYVVHLGNSKELMEVAEDNVARRVLREHIRESLGVRSEDLLFAIINSVSRGKGQDLFLQAFYQALQLIQHEKLKVPRIHAVVVGSDVNAQTKFETQLRDFVVKNTIHDRVHFVNKTLAVAPYLAAIDVLVQNSQGRGECFGRITIEAMAFKLPVLGTAAGGTTEIVLDGSTGLLHPAGKEGVAPLAKNIVRLASHAEQRVSMGEKGYGRVKEMFMEHHMAERIAAVLKDVLRKSQEHSRS

SEQ ID NO: 46 - protein, 400 aa, Zea mays
PRVRLIIEKNRDYTVDYSSSSFGLSGASYISSPMRETEQSKTSFDQFYSNANFQLYLSFCNFDKAMFLGFFHELSELPFELQRKAVRDLKTSLSGENEIWHSMVYNGFFEAFHEFLKNDSGIHTLQARRAGIQFFLAFLSSGRARIPSVCEDVVLLIASLHDSEFKQEALLIVHELLQEPSCPKSSLMASILSPSVFGALDSGETKCLDLALQIICKISSDNDIKSYLLSSGIVSRLSPLLGEGKMTECSLKILRNLSDVKETAGFIIRTGNCVSSISDHLDTGSHSEREHAVVILLGVCSHSPEVCSLSMKEGVIPALVDLSVSGTKVARDCSVKLLQLLRNFRRCDQFSSSCSRELAVDHVSENTRNGSICMQPISKSARYISRKLNLFSKPRSLTLA

SEQ ID NO: 47 - protein, 149 aa, Zea mays
MGQIESQVTPPAEEPSPPTVEPSPSSPAPPPSSLEAIAAEAMSFDEDDTEESIDVKVQKALDCPCVADLKNGPCGGQFVDAFSCFLRSREEEKGSDCVKPFITLQDCIKANPEAFSKEILEEEENDEEADKSNLKVRAPSWSRESKPKV

SEQ ID NO: 48 - protein, 45 aa, Zea mays
GQTRWEMFDLLSSLPSTSSASSTTTVSSTASSGAPPPSRPDWMLF

SEQ ID NO: 49 - protein, 217 aa, Zea mays
HASAIGRLISPSSGCSGTSSPFPDPEMQASSRSALRLFPVREPPKILDGEGVATQKLIPRHMRNGGSLLDGQISAAVPVVDFSARLQPNEHAMDHRVSFELTVEDVARCLEKKTAISGDSGTASFHLAPTGSGDHHRESNEARAGLYVDESYHDLPEKARRSLSLRLAKEFNFNNVDVGSVEPSVGSDWWANEKVAGMTTEPKKNWSFHPVVQPGVS

SEQ ID NO: 50 - protein, 198 aa, Zea mays
MGKAKKGPKFAVMKKIVTSKAIKSYKEEVLNPEKKNLMKEKLPRNVPTHSSALFFQYNTALGPPYRVLVDTNFINFSIQNKLDLEKGMMDCLYAKCTPCITDCVMAELEKLGQKYRVALRIAKDPRFERILCTHKGTYADDCLVERVTQHKCYIVATCDRDLKRRIRKIPGVPIMYITKRKYSIERLPEATIGGAPRI

SEQ ID NO: 51 - protein, 265 aa, Glycine max
MSVIDILTRVDSICKKYDKYDVQSQRDSNLSSDDAFAKLYASVDADIEALLQKADTASKEKSKASTVAINAEIRRTKARLLEEVPKLQKLAMKKVKGLSSQEFAARNDLALALPDRIQAIPDGTPAASKQTGSWAASASRPGIKFDTDGKFDDEYFQQTEESSGFRKEYEMRKMKQDQGLDMIAEGLDTLKNMAHDMNEELDRQVPLMDEIDTKVDRASSDLKNTNVRLRDTVNQLRSSRNFCIDIVLLIIILGIAAYLYNVLKK

SEQ ID NO: 52 - protein, 107 aa, Zea mays
HASGFLKCAILTKPQSYGVLLQLPAPQLENALSKNPTLKTPLAEHAEQPNIRSTLPRSTLVVLGLAEDQPQQPAVTQVQSSQNQAAETSSSAADTATEVTQESSGAS

SEQ ID NO: 53 - protein, 478 aa, Glycine max
IRLEFESLEIEMANENEPAKLLLPYLQRADELQKHEPLVAYYCRLYAMERGLKIPQSERTKTTNALLVSLMKQLEKDKKSIQLGPEDNLYLEGFALNVFGKADKQDRAGRADLTTAKTFYAASIFFEILNQFGAVQPDLEQKQKYAVWKAAEIRKALKEGRKPTAGPPDGDEDLSVPLSSSSDRYDLGTTENTVSSPGPESDSSRSYHNPANYQNLPSIHPAAPKFHDTVNDQHSANIPSSMPFHDRVDNNKHSSVVSPSSHSFTPGVYPSQDYHSPPPSRDYHSPPPSQDYHSPPSSQDYHPPPPSQDYHPPPSQDYHPPPARSEGSYSELYNHQQYSPENSQHLGPNYPSHETSSYSYPHFQSYPSFTESSLPSVPSNYTHYQGSDVSYSSQSAPLTTNHSSSAQHSSRNETVEPKPTTTQAYQYDSNYQPAPEKIAEAHKAARFAVGALAFDDVSVAVDFLKKSLELLTNPSAGQ

SEQ ID NO: 54 - protein, 143 aa, Zea mays
MEVFGKSVIAEPSNVIFLSAILNTEGSNPSHKCDKRCQSERILGNMYRCKLTETTHICDKNCNQRILYDNHNSLCRVSGQLFPLSPLEQQAVRGIRRKHEVDSSEGCCFKRRRGAQLHPSPFERSYSAVYPIPSQVGDGMDMS

SEQ ID NO: 55 - protein, 393 aa, Zea mays
MKVLVLAVLALVAAASAAGQGEEGGGPPLPFALGAAPAGCDVAQGEWVRDDDARPWYQEWECPYIQPQLTCQAHGRPDKAYQSWRWQPRGCSLPSFNATLMLEMLRGKRMLFVGDSLNRGQYVSLLCLLHRAIPDGAKSFETVDSLSVFRAKNYDATIEFYWAPMLAESNSDDAVVHSADDRLIRGAPMDRHYSFWKGADVLVFNSYLWWVAGDKIQILRGADNDPSKDIVEMKSEEAYRLVLHQVVRWLERNVDPGKSRVFFVTASPTHTDGRAWGDDDAEGSSNCYNQTSPISAASSYRGGTSREMLRATEEVLATSRVPVGLVNITRLSEYRRDAHTQTYKKQWVEPTAEQRADPRSYADCTHWCLPGVPDTWNELLYWKLFFPSNDQVL

SEQ ID NO: 56 - protein, 109 aa, Zea mays
TRPCNSMNMQLSQLPLDCKRLTYDALEGANVTPTSFYNIGDLEIQDNLARVWVDIGIHEPLLLDILLNALTTISSDHVGIKQVQFGGSEFLNWSEDLKTEEVGYSVCKI

SEQ ID NO: 57 - protein, 200 aa, Glycine max
FGSSMASATRLVHCELRSARPAVRAREPAGPVQVTIPKPKAAEAEGANIVLQPRLCTLRSYGSDRAGVLIKARKEGDDDDVSPFFAALSDYIESSKKSHDFEIISGRLAMMVFAATVTMEMVTGNSMFRKMDIEGITEAGGVCLGAVTCAALFAWFSSARNRVGRIFTVSCNAFIDSVIDQIVDGLFYEGDDPTDWPDEP

SEQ ID NO: 58 - protein, 285 aa, Zea mays
PRVRCPTTLKRICRQHGINRWPSRKIKKVGHSLKKLQMVIDSVHGSEGTVQLSSLYENFTKTTWSERELQGDATYPLSEEKGPLEPSVPDRYCEGRFTSHTSGSNSLSPSCSQSSNSSHGCSSGSKSQQHVSAPQLAVKKEVFMEENQSSTLLKAASHAELQMLPEERLVTLPRSHSQVLLSEQKPVENITGMQMSKPDSLKIKAMYGEERCIFRLQPSWGFEKLKEEILKRFGIAREVYVDLKYLDDESEWVLLTCNADLLECIDVYKSSSTQTVRILVHSSDQ

SEQ ID NO: 59 - protein, 115 aa, Zea mays
MRAMSSAVNGMLRARLRGAARVRGGGGEGAGRWTTPGHEERPKGYLFNRPPPPPGESRKWEDWELPCYVTSFLTVVILGVGLNAKPDLTIETWAHQKALERLQQQELAAADTQAE

SEQ ID NO: 60 - protein, 168 aa, Glycine max
IRLEQRPVEGNATMKQDLRSFKLILEYIKALPTGQETDFVLVSCSGLGIEPSRREQVLKAKRAGEDSLRRSGLGYTIVRPGPLQEEPGGQRALIFDQGNRISQGISCADVADICVKALHDTTARNKSFDVCYEYIAEDGRELYELVAHLPDKANNYLTPALSVLEKNT

SEQ ID NO: 61 - protein, 723 aa, Zea mays
MAGKEDENEKPSLVAAGGKQDRTAATTESLPQRTNLEWGKAACSEDDIQKCVAAGAFHPGELVEWRAPVKDETPTLSTMEDQFVILSLTHIICGLRVDASDFLVSVLEYYRLEWSHLTPNSITALSIFAHLCEAYVEAPPTVEVFTHFYSLYHNRKGETTTLGAVYFRLRDRMKKNYPLYYLRSSQFMWVSLWFYAKVPKSCRLTFRGDILKEENNWNWKDLLPLSCEQMKQVGQIMKLSNQGLTGADIIHDYLKRRISPLRRRMHLTCNYSGLSDPTRDSDKDLSVEDIESKLSYLLDLKRMGVKQPTGRLVRASTNDQANQPLDLLNVCSTHEAKKEAQPQVCASLRRYTRQSAGPRKVAVPPPLKIDPPPTQGPAPEEILDATTNIATAVSPNLGVEQKSIEHPTVAEERKIAELVKPTFSVIGAKRKASAPRSRSKRRAKYSLLSVVTKTRMSSLDTGSIKGTSRTKEAVLALNSRSIGLARCPPASLAEGTGNGGLFMLAKVVDHTKVVEDSMSNILLNQQVGDSSRKEVDPAQSITEIVQDQEAIEVSAAIPVPNKEEINSGVIWERMQKVQSEYVSLSMTASSELLEQAKKLVMENKRLKDVQIMLSQQVKDLEDGRRLLTERMKKAEQETFKIIEENMKLKDENKGQKQMIEELSKQNESTLGALVHKCTLLDRYKEESAQLIREKEELQSRVSRVNDLVKLVSSTLCQEKDSAS

SEQ ID NO: 62 - protein, 342 aa, Glycine max
MSEKALRDLNTILGTERKNEDSSKACLSKPSVDNAVENIEEWQKKNNSPSLVSPAVNGNLAVTANSGAEVVNPEVEYIESENLNDVDDIDTCLKTLLAGLDSKDWVLVCDTLNNVRRLSIFHKEAMLDMLGDVITSIAKSLKSPRSAVCKTAIMTSADIFSAYNDLIIDSLDPLLVQLLLKSSQDKRFVCEAAEKALISMTIWISPISLLPKLQPYLKNKNPRIRAKASMCFSRSVPQLGAEGIKTYGIDKLIQVAASQLSDQLPESREAARTLLLELQNVYEKSHDLIKPATPTVNNEHTVNEENPEVSSWESFCQSKLSPLSAQAVLRVTTSIAREGLVS

SEQ ID NO: 63 - protein, 509 aa, Zea mays
PRVRALLASTIVPHPNQGNMHEPAIDMPFGSVLLQALVSSDVNGDMEACCRASSVLSHIVKDNMQSKDRVLQIQLETLTPSLGRTEPVLHRIVTCLSIAASTEGENNQNNQPEEPYIQPVILRLLIIWLVDCSNAVNCLLESAVHLNYIIELASSKRYTACVRGLAAVVLGACILYNASHEKGRDAFAVADAISQKIGLTTYFLRFDELRRSLAHPLPEQHHRKELSRSSANSMSDFQEIEEDETNKDDQHPVLSEIFDSQFVNFLSKLEADIRENIMDIFSRTKTATALLPTELEQKNGEVDGEYIKRLKSFVEKQCNEMQDLLARNAMLAEELVRTGGGTTTDTSQRPNNGRERVQIEALRQELEGARRQIEALETDKSQIEAEANNQRNLAVKLESDLKSLSEAYNSIEQANYRLDAEVKTLRQGGSVPYPDVEAIKAQAKEEAEKDSEAELNGLLVCLGQEQTKVEKLSTRLAELGEDVDALLQGIGDDTAIPDDDDDDDEDSEE

SEQ ID NO: 64 - protein, 102 aa, Zea mays
QFKIDPQDFQDSEPDILANSASSIIERIKENSDQCAAALRSLCRRKKGLTVEEASLIGVDSLGIDVRAFSGLEVKTVRFSFNAQALSERSAEKKIRRMLFPR

SEQ ID NO: 65 - protein, 587 aa, Zea mays
PRVRSVLKTKPSPRILTEAAPWRQQERSATNICREAEGRPRIASVYADIERRVGGFDFLECNNKDFRALRILGALNARDAKNKNDSSGRPMATHRTGYDLTTSGSFQAPIVVMKPAGTTEKHGVSLASVAPIAGLRSLRKLPARYSSFTGTNETSTNENIHLRMSRAQLKSEETVSSANSPRPTSSSSPRNVLKNAEPERRSRPPVSPKSPSKKSNEVVSPKGRTRSKPSQVKSHRDEVLQSTGNRISLAKQVDVSIIDCPKLPGGNSTFVPPSNAAATASHKAPSILDSDQNIHSLDNIPSPVSVLDTSFYHKRISDSFKDGETHSSEECWNPNSLPDTPQSKASSEANQIKPENLEVLIQKLEQLQSMNEEDASIKEVMASVTANKDHQYIYEILLASGLLHKEHSITALPAQLQPSNYPINPELFLILEQTKPDLVFAFQTVSGTKKSCKPYTGKLHRRLVFDLVNETIAQKMIICRSGSQPVKFLQSRKLSGWQLFKDLCTEVDRQIKCTGEEENGNMILDEDTVNGTKDWMSFDTMLHGMVWEIERSIFKGLIDEVIGGETIEKMQFGQRKLQRQLSFSSIN

SEQ ID NO: 66 - protein, 243 aa, Zea mays
PRVRIFLAWWLTRKAQIHCLAVQMLLLRCLLMDLDRQNTGKATVLGDAARVLRDLITQVESLRQEQSALVSERQYVSSEKNELQEENSSLKSQISELQTELCARMRSSSLSQTSIGMSDPATHQQMQMWSSIPHLSSVAMAARPASAASPLHGQEGYSADAGQAGYAPQPQPRELQLFPGSSASSSPERERSSRLGSGQATRPSLTDSLPGQLCLSLLQPSQEASGGGGGGVMSRSREERRDG

SEQ ID NO: 67 - protein, 423 aa, Zea mays
TRPAIRELWRPNPSQLILLQTRGIGALHKELPKACALTGSSDPCYIEAYHLADPTDGRITLHLKILNLTELELNRVDIRVGLSGALYYMDGFSRTVRHLRNLVSQDPVQSSVTVGVSHFERCSLWVQVLYYPFYGSSGSTDYEGDYAEEDSQMMRQKRSHRPELGEPVVLRCQPYKFPLAELLLPLECSPVEYFRLWPSLPAMVECTGTYTYEGSGFKATAAQQYDSSPFLSGLKSISSKPFHQVCSHFIRTVAGFQLCYAAKTWFGGFVGMMIFGASEVSRNVDLGDETTTMICKFVMRASDESITREIKSDLQGWLDDITDGAVEYMPEDEVKSVAAEQLKISMERIALLKAARPKVPPAKTDQEEEEERKQSEELDGFGNPKGPSTLSKLTAEEAEHRALQAAVLQEWHQLCKERAMKAQ

SEQ ID NO: 68 - protein, 371 aa, Zea mays
PRVRASGISGTSVRLTAGAGLPVHMKGELNTAFIGLGDDGGYGGGWVPLAALKKVLRGILKYLGVLWLFAQLPELLKEILGSILKDNEGALLNLDQEQPALRFYVGGYVFAVSVHRVQLLLQVLSVKRFHHQQQQQQAQSNAQEELAAVEINEICDYFSRRVASEPYDASRVASFITLLTLPILVLREFLKLITWKKGLSPVHGDIATAQRARIELCLENHSGSASSDNTENSSLAKSNIHHDRAHSSVEFALTFVLDHALIPHMNVAGGAAWLPYCVSVKLRYSFGDNNHIAFLAMNGSHGGRACWLQFEEWERCKQKVSRAVETVNGSGVAGEVGQGRLRMVAEMIQKQLQLCLQQLRDDPLSAGSTAS

SEQ ID NO: 69 - protein, 178 aa, Zea mays
TRPDDPCPYLLSIWTPGETAQSIDAPKTFCDSGETGRLCGSSTCFSCNNIREMQAQKVRGTLLIPCRTAMRGSFPLNGTYFQVNEVFADHCSSQNPIDVPRSWIWDLPRRTVYFGTSVPTIFRGLTTEEIQRCFWRGFVCVRGFDRTVRAPRPLYARLHFPVSKVVRGKKPGAARAEE

SEQ ID NO: 70 - protein, 222 aa, Zea mays
TRPLRVKKLQQEAARCLSLHKTMELQPELSLGPVWPGFAAGDLAAKSSSSESDGTSRKKRKHYTASWEEPQQPPASLELQLNDPLPLDWEQCLDLQSGRMYYLNRKTLKKSWVRPQVQSVNLDLNISTAAAIDNCAANGAAAAASDDEDEPRKPAGTLFSGGSMVAVPCANCHLLVMLCKSSPSCPNCKFVQPLAPAVPPAAVAHWRIDAAVKPLETLSLLH

SEQ ID NO: 71 - protein, 303 aa, Zea mays
MKLKNSAVETFKENNMIFTSEGNLHSKKMREDYVASPNQPGAVQTRCKWIIGDVTEVFDRSTWKLGKILKMLKNNYFVIRLADCIQLKEFHISSLRIPRGLEAPQSKPFHAADKATGRGNRRPADGALPGARAADQMGHRAYELGSSGKKRKATADASHHLGRAAAAHSRKVAAASNPNGGSYPHSSSQAIEDAECSVASCSVDDLYRLGNGGNAKRRPAAAGCLPDDAMSACPCTPGARDGEDDDAAGVHGLELEAYGSTMRALYASGPLTWEQEALLTNLRLSLNISNEEHLLQLRRLLSS

SEQ ID NO: 72 - protein, 336 aa, Zea mays
PRVRRHARHEHDRSGDWSHRRRAGRGRIPPLRLLSFDSLLPASNPSHHPFLPMASDAPAEQPATQQKPTRVSLSYEEISKLFSLPIAEAASILGVCTSVLKRICRTHGIVRWPYRKLVSGKAGDDTKGPDSDKANELLEVSKIAKQKAPSASGPSVVSSSTSQGAAKSQQGNSKAGQFSVSPPTGKHNASLSLTHSQAKAIPCYMDDFKYGFPSSGLSCETMKWWGTSSDTDYVPTKDGSHEPHESTTHEPSKGMTDDDELDWGADEAEAEADGTVTAEASAQLCSLRRKAVDDGRKLLNGHNRRGQEFSRLNKRQKTALAQVFGASLPECCITRV

SEQ ID NO: 73 - protein, 196 aa, Zea mays
EIDPITKEVLATPIADALGRKFTRFGHQAKEDRQAAIFRSENGNVWQVKIFGEDKTGKRSGQYLAPTGIGDVPYLPTIPRRIILAIAEKHGVKPPEDGQDFWPWFVDHPEIPLIVTEGGKKALAAISQGYVALSLYGCLCGNDGLTIKPSLLPYVQGREVAIAYDQDAKGSKGRKAVFKGTKRLARNLTYHAKATV

SEQ ID NO: 74 - protein, 151 aa, Zea mays
PRVRMNLRRQTPLAAIHAALASADAMVAVHGAAVTHFLFMRPGSVLLQVVPVGLDWAADAFYGKPAQQLGLEYLEYKVAPEESSLAAEYGLDSTVLRNPWVISSRGWWEMKKVYMDRQNVTVNIKRFGELLRTARTHLKNTTACAAAAALR

SEQ ID NO: 75 - protein, 356 aa, Zea mays
TRPIEPGSRPETSDYPQSSERPLTATSSFSSASPFSESSQLASSSKQPAPYLPRNHMGRRSFMSKPVYPLVFRNPVSESEACRMLEVGNAGRATPSDDSQASPLWRRSLASPDLKFHNAPNELGKMETSPEPNTSSRREGFRWSNASSYDFGYDGDAIDISDHISIESQRSPTSSARFLKCGLCERFLHQKSPWTSNRIVRNADMPVAAVLPCRHVFHADCLEESTAKTEVHEPPCPLCARATDDEGHVSFSEPLHVALRSARRNLSLGTGAGGNSGISDPPRTDRGLKRNNSAVMPRRSGGALFRNRFKKQFPFKARIGKELFGGRVLNKVGLSLSSGQHDDHRQQAPKHDRPMK

SEQ ID NO: 76 - protein, 940 aa, Zea mays
MAMAMARFLSWLFTCFAALAVLEATVPARSWRAPSPTPRHEARRFEQKTDRFWEYQEQSNTWVQVRAPFDLMSCINGTCTKVGSIGRLAREPGRHGLPPVQSQEEEEEDTRRVQGDGAEEDPVLPVRRRISLTRMSESSVWVTGQSGSIYERFWNGVVWVIAPHELPASAGYATATFIVNTTILALSEAGTLYQLQLNEHAQPIWTEMAFNSSQQSANLGLKTQSQAMRIRNGIVSNDGRKLFLSIMNGSLLEVTEIQPLRWNYHGRPPGADVSYISDAGNLRPGTLFTVSSTGDLYEFDKETKPSWKKHIWSEELAKNISLKSSAGFALHGLSGSNSVSLFLISKDGLLVERRLHRRKWKWDKHGAPTGQRLSSIAEVQKDELNDATSMFLTTTTGKVYEYQFPKYTGGAQSNKIRGQWINHMSPEHAKVARNVPGVHVQVGRMVFPLDDGRLGELHFPGMGGTDFGPSAQSTIRRKLSNKYEWSILDAPETEGWNAEYCTEEHGPTNCISGAKNIAADTESNDLSNNPPSRRRKVEEKQHYLNVNRYQQSDETESYNFLSRTIDLNFHMRVMHADRSLFLIADNGLTFEYLNSNGVWLWLRHEHVTAMKGTLGSYNGSLYLVDVHGNLHIRERNGDELLWINCTAMKKGRQVASGSPWDGIPGLLRRVTTDDALFFVNKRGRLLQFTVALRKFKWKDCHSPPDTKIAFIVDQEVFRRNIIFVVGRNGRLYQYNRITELWHRHYQSPHLFLSCSPGTAMRPSPLSLAGSLFMVSEHGGLVEYHFSPQDGWEWVEHGTPHRGVTLVGAPGPCFDGSQLFVVGSDGHVYRRHMEGRTWRWTSHGHPPSEPAAVDEQSCATPDTGAGAHYADGFRGSCDGKVAAVRPVPFSEDAVVFELRDGRLAELRRPPSADGCGGWEWARIIGTPASACMTSYWTAVAT

SEQ ID NO: 77 - protein, 556 aa, Zea mays
HASGTMEIGLRGPTNLFGHPTDKQMIELDQALSQWNTDFDKVPVTKIAFGHFPLSFSALTESGKSIKDVFLKQSLAAYLCGHLHTRFGKNLKRYYHRAVQESSLSEHYYQHNMHQGDAFQGNKENCSEEASHIEEFWEWEMGDWRKSRSMRILAIDDGYVSYTDIDFRLGSKSIIILPTFPLDSRFMQRASAFRDFKCHVMGASSFDTVRALVFSKHEIISVSVKIYDSRPGTLEIVFDSEMKRVDSNETRGNMYLIPWNWRAFEDSSPSRYWLQIEVMDITGDTSVSQLRPFSVNGLPARVNWTWKEFFVIGIQWASIYHPALWCAFSLIFSLLLVPQVLAVVFKDRFTYKSLCAYGGQRTLLKSLVGGFICCFVELSRLVLVWLLLLLYAIYLVFIPWLFGHPITEDGSLTYMTHKGWILKGPSSSNEVVHAGIPDVMVIVLPHLCFVLVPTIVILAAMAAERTAYREHYLSRSGKKKDDYRKSRTQIEHENFWNGRWISKFLCLLCVVVLCKHWKLCRALVKAYAMNPLLHAPVLFFFVPLLMVFAIYKTRSI

SEQ ID NO: 78 - protein, 167 aa, Zea mays
MAGAEGERWVGLATDFSEGSRAALRWAAANLLRAGDHLLLLHVIKEPDYEQSEAILWESTGSPLIPLSEFSDPIIAKKYGAKPDIETLDILNTTATQKDIVVVVKVLWGDPREKLCQVIHDTPLSCLVIGSRGLGKLKRVLLGSVSDYVVNNATCPVTVVKSTSTEG

SEQ ID NO: 79 - protein, 81 aa, Zea mays
QTRAYLSNVCVAKELQKKGLGYTLVDKSKKLALEWGITDLYVHVAINNVAGQKLYKKCGFVYEGEEPAWKGRFLGRPRRLL

SEQ ID NO: 80 - protein, 291 aa, Zea mays
MNGGLPGFHNAPASKAVVVAAGLFSVAFGFRGHSLNLGLAYQSVYEKLSVWRLITSFFAFSSTPELIFGAVLLYYFRVFERQIGSNKYAVFIIFSTMVSVLLQILALGYMKDPSLNPLTSGPYGLIFASYVPFFFDIPISMKFRIFGLSFSDKSFVYLAGLQLLFSSGRRSIVPGLSGILAGLLYRLNTFGVRRLKFPEFATSLFSQLSLPFSSNPYQGLPITENDGSIPSHQARQIEDARTATQDPTESSIAALVSMGFDRSAAIQALALTNYDVNLASNILLEAQALQQ

SEQ ID NO: 81 - protein, 294 aa, Glycine max
MDSSCVPNGDVSGFKDKEPMVDPFLVEALQNPRHRVTILRMELDIQRFLNNADQQHFEFQHFPSSYLRLAAHRVAQHYGMQTMVQDNGFNGQGTRIMVRKIAESRYPVVCLSEIPAKQLEDDKPEQIKIAIRPRQNKNSLNEAGRKSNPLRSVEERKEEYDRARARIFSSSRSCDSDDTLSQTFTDEKNSLIIKDENETSKTPVVDSEQCTVGRDISSTRVAILRDREKDRSDPDYDRNYGRYARSIPISSLNLMPFNLQQVQPPFVQYDNALIRSVRYHKIKLHLAMDLLQAL

SEQ ID NO: 82 - protein, 238 aa, Zea mays
ARGSAHYRTFWVTDSHYLTATGPAIAIFTNPTKQGYDDGLGEKIIGTFGNCAGGTTPWGTVLSAEENFQSQVPEAVYADGSAVDPAQCPLKISTNGLSGQGNVFGLAGNKYGWMVEIDPANANDYGVKHTALGRFRHEAVAVRATANQPLAVYSGCDRTSGHLYKFVSADTVKSPTDKGNSRLFTAGTLYGAKFNADGTGEWIALTPDTVVNPVRPSDIAVDSSTTGIVYLPHPDRNQ

SEQ ID NO: 83 - protein, 322 aa, Zea mays
PRVRVPLHRMSDPAAGGAMVPAAGRGIAWANGGPRFGDMVWAKVKSHPWWPGHIYSVSLTDDEEVHRGHRDGLVLVAFFGDSSYGWFDPSELVPFEDHFTEKAAQGGSSRSSFAAAVAEAVDEVARRSALALLCPCDIPDAFRPHPSDGNFFLVDVPAFDTDADYQLDQIRAARQRFVPRKALNYLLDAAVTQRDAAEKAARTVPGMEMAALFLAYRRAVFSPIDNTYAQAFGVDPELALAAEQKAAAERAQRGINNTHMLNSSCTVVLVTVYLKLMGKQCSYCFYRSSNNYFDVNNGVDLIQIVSISLLFNCWLHFFYQRE

SEQ ID NO: 84 - protein, 187 aa, Zea mays
HASEASAAHLTNYGNMVSAQERSIQHTAYNPEVTLNLPPPPPLPTIPHSSATLQSQGGHSLPSQTNQQLYQPEQYYVPQNNYGPLVPVSHSNLQISNTNNPTLTIPQVNPGPPTNNQIGNLAQPQHSMPLHVDRASQDFSSQGQQQNRGPGAAQAPEEDKSKKYQATLQLAQNLLLQLQQRGSGNQS

SEQ ID NO: 85 - protein, 258 aa, Zea mays
HASDPTEFILETLEQSDPQSLIQYLAYQDLCVVSECNLEPWRRAAFFEESGETYRRIVTACLKPLEEFTSKIAEALEGFSSDQPELMLQQSRLFSAFDDSQICTWCARTLAGLTARSRKEDRYGVAQLTGCNAAVMTTLLSALVAIETCLGKKTNPQPVRSLGPENIRWTNLSTGRKGNGVAIASTQKSGLHKKAYIMADVLRTSVYHILSAFIDDLQANAKPSSLEKNWISEGRKPVYGSQAVLVQKLILFIEYRAV

SEQ ID NO: 86 - protein, 288 aa, Zea mays
GLEEEDGEEAAPASPWAEADAQAGGAEAQTEVLGAGEPDLESKIVAIRDFLEDPNQPENELVSLLQNLADMDVTYNALQETDIGRQVNGLRKHPSAEVRRLVKQLIRKWKEIVDDWVRLDNSGGDGSASVMTDGDSPHKIQGRSHQSPRVSGFQYSPSPQRFNGSTSEMANNGFESTMDAKRRASPVPAHHNSRQMNNNHHSTTITTSTSSAPAFSVQKVTREQKQSLVDLDRLDSARKRLQENYQEAQNAKKQRTIQVMDINDIPKPKSRNAFIRKSGSGGLPARHR